(19) United States
(12) Patent Application Publication
(10) Pub. No.: US 2024/0325396 A1
(43) Pub. Date: Oct. 3, 2024

(54) METHODS FOR TREATING CANCER

(71) Applicant: Allarity Therapeutics, Inc., Boston, MA (US)

(72) Inventors: Marie FOEGH, New York, NY (US); Steen KNUDSEN, Melby (DK)

(21) Appl. No.: 18/606,590

(22) Filed: Mar. 15, 2024

Related U.S. Application Data
(60) Provisional application No. 63/602,480, filed on Nov. 24, 2023.
(60) Provisional application No. 63/452,795, filed on Mar. 17, 2023.

(51) Int. Cl. A61K 31/519; A61K 9/28; A61K 9/48; A61K 31/496; A61P 35/00

(52) U.S. Cl. CPC A61K 31/519; A61K 9/28; A61K 9/4825; A61K 31/496; A61P 35/00

(57) ABSTRACT

Described are methods of treating cancer (e.g., solid tumor cancer) using stenoparib monotherapy at BID dosing or a combination therapy including stenoparib (at BID dosing) and dovitinib (at QD dosing).

### SEQUENCE LISTING

The instant application contains a Sequence Listing which has been submitted electronically in XML format and is hereby incorporated by reference in its entirety. Said XML copy, created on Mar. 13, 2024, is named 51167-027003_Sequence_Listing_3_13_24 and is 418,920 bytes in size.

### BACKGROUND OF THE DISCLOSURE

Cancer is responsible for about 25% of all deaths in the U.S. and is a major public health problem in many parts of the world. Estimates are that in 2018, 18.1 million new cases of cancer and 9.6 million deaths occur globally.

Stenoparib (also referred to as 2X-121 or E7449) is an orally bioavailable, brain penetrable, potent small molecule inhibitor of poly (ADP-ribose) polymerase (PARP) 1 and PARP2 activity that has been used for treating cancer.

Dovitinib (also referred to as TKI258), a pan-targeted kinase inhibitor (pan-TKI), is an inhibitor of type III-V receptor tyrosine kinases (RTKs) that mediate both endothelial and tumor cell proliferation and survival.

There exists a need for improved methods for treating cancer.

### SUMMARY OF THE DISCLOSURE

In one aspect, featured is a method of treating a cancer in a subject in need thereof including administering to the subject a dosing regimen including a first dose and a second dose of a composition including stenoparib or a pharmaceutically acceptable salt thereof or a hydrate thereof per day.

In some embodiments, the dosing regimen includes a dosing cycle including administration of the composition for 28 days±1 to 5 days.

In some embodiments, the dosing regimen includes more than one dosing cycle.

In some embodiments, the dosing regimen includes 2 to 100 dosing cycles.

In some embodiments, the dosing regimen includes 2 to 8 dosing cycles.

In some embodiments, an amount of stenoparib or the pharmaceutically acceptable salt thereof or the hydrate thereof administered in the first dose is less than or equal to an amount of stenoparib or the pharmaceutically acceptable salt thereof or the hydrate thereof administered in the second dose.

In some embodiments, the first dose includes between about 200 mg to about 400 mg of stenoparib or the pharmaceutically acceptable salt thereof or the hydrate thereof (or any dosage between 200 mg and 400 mg).

In some embodiments, the second dose includes between about 400 mg to about 600 mg of stenoparib or the pharmaceutically acceptable salt thereof or the hydrate thereof (or any dosage between 400 mg and 600 mg).

In some embodiments, the first dose includes about 200 mg of stenoparib or the pharmaceutically acceptable salt thereof or the hydrate thereof and the second dose includes about 400 mg of stenoparib or the pharmaceutically acceptable salt thereof or the hydrate thereof.

In some embodiments, the first dose includes about 400 mg of stenoparib or the pharmaceutically acceptable salt thereof or the hydrate thereof and the second dose includes about 400 mg of stenoparib or the pharmaceutically acceptable salt thereof or the hydrate thereof.

In some embodiments, the first dose includes about 400 mg of stenoparib or the pharmaceutically acceptable salt thereof or the hydrate thereof and the second dose includes about 600 mg of stenoparib or the pharmaceutically acceptable salt thereof or the hydrate thereof.

In some embodiments, the second dose is administered to the subject at least 11 to 13 hours after administration of the first dose.

In some embodiments, the cancer is a solid tumor cancer.

In some embodiments, the cancer is an ovarian cancer, a breast cancer, a colon cancer, a pancreatic cancer, or a prostate cancer.

In some embodiments, the cancer is locally advanced or metastatic.

In some embodiments, the cancer includes a BRCA mutation.

In some embodiments, the cancer is an ovarian cancer, and the ovarian cancer is an epithelial ovarian carcinoma. In some embodiments, the epithelial ovarian carcinoma is a high-grade serous epithelial ovarian carcinoma, an endometrioid epithelial ovarian carcinoma, a clear cell epithelial ovarian carcinoma, an epithelial ovarian carcinosarcoma, an undifferentiated epithelial ovarian carcinoma, or an epithelial ovarian carcinoma of mixed histological subtypes.

In some embodiments, the cancer is homologous recombination (HR) deficient.

In some embodiments, the subject is non-responsive to at least one cancer therapy other than stenoparib or a pharmaceutically acceptable salt thereof or a hydrate thereof. In some embodiments, the subject is non-responsive to at least two cancer therapies other than stenoparib or a pharmaceutically acceptable salt thereof or a hydrate thereof. In some embodiments, the at least one cancer therapy comprises a PARP inhibitor.

In some embodiments, the subject is administered the composition orally.

In some embodiments, the composition is formulated as a capsule or a tablet.

In some embodiments, the composition is formulated as a capsule, and each capsule includes about 50 mg, about 100 mg, or about 200 mg of stenoparib or the pharmaceutically acceptable salt thereof or the hydrate thereof (or any dosage between 50 mg and 200 mg).

In some embodiments, the method includes, prior to administration of the composition, determining the responsiveness of the subject to treatment with stenoparib or the pharmaceutically acceptable salt thereof or the hydrate thereof by determining the expression level of one or more biomarkers of sensitivity selected from the biomarkers of Table 1; and/or determining the expression level of one or more biomarkers of resistance selected from the biomarkers of Table 2.

A second aspect features a method of treating a cancer in a subject in need thereof including administering to the subject a dosing regimen including administering a first dose and a second dose of a first composition including stenoparib or a pharmaceutically acceptable salt thereof or a hydrate thereof per day; and administering a dose of a second composition including dovitinib or a pharmaceutically acceptable salt thereof or a hydrate thereof.

In some embodiments, the dosing regimen includes a dosing cycle including 28 days+1 to 5 days.

In some embodiments, the dosing regimen includes more than one dosing cycle.

In some embodiments, the dosing regimen includes 2 to 100 dosing cycles.

In some embodiments, the dosing regimen includes 2 to 8 dosing cycles.

In some embodiments, the method includes administering the first and second doses of the first composition each day of the dosing cycle and administering the second composition on a 5 days on and 2 days off schedule during the dosing cycle.

In some embodiments, the second composition is administered to the subject as a single dose on Days 1-5, Days 8-12, Days 15-19, and Days 22-26 of each dosing cycle.

In some embodiments, an amount of stenoparib or the pharmaceutically acceptable salt thereof or the hydrate thereof administered in the first dose of the first composition is less than or equal to an amount of stenoparib or the pharmaceutically acceptable salt thereof or the hydrate thereof administered in the second dose of the first composition.

In some embodiments, the first dose of the first composition includes between about 200 mg to about 400 mg of stenoparib or the pharmaceutically acceptable salt thereof or the hydrate thereof (or any dosage between 200 mg and 400 mg).

In some embodiments, the second dose of the first composition includes between about 400 mg to about 600 mg of stenoparib or the pharmaceutically acceptable salt thereof or the hydrate thereof (or any dosage between 400 mg and 600 mg).

In some embodiments, the dose of the second composition includes between about 300 mg to about 500 mg of dovitinib or the pharmaceutically acceptable salt thereof or the hydrate thereof (or any dosage between 300 mg and 500 mg).

In some embodiments, the first dose of the first composition includes about 200 mg of stenoparib or the pharmaceutically acceptable salt thereof or the hydrate thereof and the second dose of the first composition includes about 400 mg of stenoparib or the pharmaceutically acceptable salt thereof or the hydrate thereof.

In some embodiments, the first dose of the first composition includes about 400 mg of stenoparib or the pharmaceutically acceptable salt thereof or the hydrate thereof and the second dose of the first composition includes about 400 mg of stenoparib or the pharmaceutically acceptable salt thereof or the hydrate thereof.

In some embodiments, the first dose of the first composition includes about 400 mg of stenoparib or the pharmaceutically acceptable salt thereof or the hydrate thereof and the second dose of the first composition includes about 600 mg of stenoparib or the pharmaceutically acceptable salt thereof or the hydrate thereof.

In some embodiments, the dose of the second composition includes about 300 mg of dovitinib or the pharmaceutically acceptable salt thereof or the hydrate thereof.

In some embodiments, the dose of the second composition includes about 400 mg of dovitinib or the pharmaceutically acceptable salt thereof or the hydrate thereof.

In some embodiments, the dose of the second composition includes about 500 mg of dovitinib or the pharmaceutically acceptable salt thereof or the hydrate thereof.

In some embodiments, the second dose is administered to the subject at least 11 to 13 hours (e.g., 11 hours, 12 hours, or 13 hours, or any time period between 11 and 13 hours) after administration of the first dose.

In some embodiments, the cancer is a solid tumor cancer.

In some embodiments, the cancer is an ovarian cancer, a breast cancer, a colon cancer, a pancreatic cancer, or a prostate cancer.

In some embodiments, the cancer is locally advanced or metastatic.

In some embodiments, the cancer is HR proficient.

In some embodiments, the subject is non-responsive to at least one cancer therapy other than stenoparib or a pharmaceutically acceptable salt thereof or a hydrate thereof and/or dovitinib and or a pharmaceutically acceptable salt thereof.

In some embodiments, the subject is administered the first composition orally.

In some embodiments, the first composition is formulated as a capsule or a tablet.

In some embodiments, the first composition is formulated as a capsule, and wherein each capsule of the first composition includes about 50 mg, about 100 mg, or about 200 mg of stenoparib or the pharmaceutically acceptable salt thereof or the hydrate thereof (or any dosage between 50 mg and 200 mg).

In some embodiments, the subject is administered the second composition orally.

In some embodiments, the second composition is formulated as a tablet.

In some embodiments, each tablet of the second composition includes about 100 mg of dovitinib or the pharmaceutically acceptable salt thereof or the hydrate thereof.

In some embodiments, the method includes, prior to the administration of the first and second compositions: determining the responsiveness of the subject to treatment with stenoparib or a pharmaceutically acceptable salt thereof or a hydrate thereof by determining the expression level of one or more biomarkers of sensitivity selected from the biomarkers of Table 1; and/or determining the expression level of one or more biomarkers of resistance selected from the biomarkers of Table 2; and/or determining the responsiveness of the subject to treatment with dovitinib or a pharmaceutically acceptable salt thereof or a hydrate thereof by: determining the expression level of one or more biomarkers of sensitivity selected from the biomarkers of Table 3; and/or determining the expression level of one or more biomarkers of resistance selected from the biomarkers of Table 4.

### Definitions

As used herein, “a” or “an” means “at least one” or “one or more” unless otherwise indicated. In addition, the singular forms “a”, “an”, and “the” include plural referents unless the context clearly dictates otherwise.

As used herein, the term “about” means+/−10% of the recited value.

As used herein, by “administration” or “administering” is meant a method of giving a dosage of stenoparib or a pharmaceutically acceptable salt thereof or a hydrate thereof, dovitinib or a pharmaceutically acceptable salt thereof or a hydrate thereof, or another therapeutic agent to a subject. The compounds utilized in the methods described herein can be administered, for example, intravenously, intramuscularly, transdermally, intradermally, intra-arterially, intracranially, subcutaneously, intraorbitally, intraventricularly, intraspinally, intraperitoneally, intranasally, or orally, or by another other route described herein. In particular instances, the compounds and compositions utilized in the methods described herein are administered orally.

The term “stenoparib” or “2X-121” or “E7449,” as used herein refers to 8-(isoindolin-2-ylmethyl)-2,9-dihydro-3H-pyridazino [3,4,5-de] quinazolin-3-one or a pharmaceutically acceptable salt thereof or a hydrate thereof. Stenoparib has the following structure:

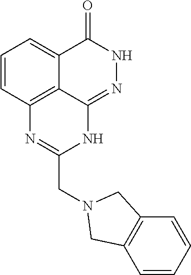 [SMILES: O=c1[nH]nc2c3c(cccc13)N=C(CN1Cc3ccccc3C1)N2]

Stenoparib has a calculated molecular weight of 317.3446 g/mol.

Stenoparib is a small molecule targeted inhibitor of Poly (ADP-ribose) Polymerase (PARP), a key enzyme involved in DNA damage repair in cancer cells. Stenoparib is an orally bioavailable, brain penetrable small molecule PARP inhibitor having novel dual-inhibitory action against both PARP ½ and Tankyrase ½ (important regulators of canonical Wnt/β-catenin, a critical checkpoint in metastases, particularly in triple-negative breast cancer). Stenoparib is not a substrate for P-glycoprotein and is active in P-glycoprotein expressing cells, suggesting that it may overcome PARP inhibitor resistance. Stenoparib was formerly known as E7449 and MGI 25036. Stenoparib is described in McGonigle et al. (Oncotarget 6:41307-41323, 2015) and WO 2009/046205, both of which are hereby incorporated by reference. Examples of pharmaceutically acceptable salts of Stenoparib are described in WO 2009/046205. Stenoparib can be identified by the CAS No. 1140964-99-3. In a particular instance, the form of stenoparib is stenoparib L-tartrate, anhydrous.

As used herein, the term “dovitinib,” “TKI-258,” or “TKI 258” refers to 4-amino-5-fluoro-3-[6-(4-methylpiperazin-1-yl)-1H-benzimidazol-2-yl]-1H-quinolin-2-one or a pharmaceutically acceptable salt thereof or a hydrate thereof or a tautomer thereof, or a mixture of tautomers thereof. 4-amino-5-fluoro-3-[6-(4-methylpiperazin-1-yl)-1H-benzimidazol-2-yl]-1H-quinolin-2-one has the following structure:

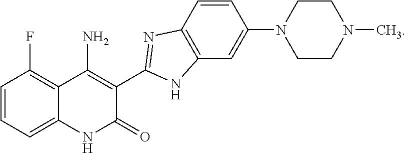 [SMILES: CN1CCN(c2ccc3nc(-c4c(N)c5c(F)cccc5[nH]c4=O)[nH]c3c2)CC1]

Dovitinib is a small molecule targeted inhibitor of multiple protein kinases (e.g., tyrosine receptor kinases (RTKs)). Receptor tyrosine kinases (RTKs) are transmembrane polypeptides that regulate developmental cell growth and differentiation, remodeling and regeneration of adult tissues. Polypeptide ligands known as growth factors or cytokines, are known to activate RTKs. Signaling RTKs involves ligand binding and a shift in conformation in the external domain of the receptor resulting in its dimerization. Binding of the ligand to the RTK results in receptor trans-phosphorylation at specific tyrosine residues and subsequent activation of the catalytic domains for the phosphorylation of cytoplasmic substrates.

Dovitinib, or a pharmaceutically acceptable salt thereof or a hydrate or solvate thereof, exists in a variety of polymorphs. Dovitinib, or a hydrate or solvate thereof, or a polymorph thereof, is useful for inhibiting angiogenesis and treating proliferative diseases. In a particular instance, the form of dovitinib is dovitinib lactate, monohydrate.

The structure and activity of 4-amino-5-fluoro-3-[6-(4-methylpiperazin-1-yl)-1H-benzimidazol-2-yl]-1H-quinolin-2-one or a tautomer thereof is described in WO 2002/022598 and WO 2012/001074, hereby incorporated by reference. Dovitinib can be identified by the CAS No. 405169-16-6. In a particular instance, the form of dovitinib is dovitinib lactate, monohydrate. Dovitinib lactate, monohydrate can be identified by CAS No. 915769-50-5.

As used herein, “dosage form” means a pharmacologically active material in a medium, carrier, vehicle, or device suitable for administration to a subject. A dosage form can include a disclosed composition or a product of a disclosed method of making, in combination with a pharmaceutically acceptable excipient, such as a preservative, buffer, saline, or phosphate buffered saline. Dosage forms can be made using conventional pharmaceutical manufacturing and compounding techniques. Dosage forms can include inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts), antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose), and co-solvents (e.g., glycerol, polyethylene glycol, ethanol). A dosage form may be specially formulated for administration in solid or liquid form, including those adapted for the following: oral administration, for example, drenches (aqueous or non-aqueous solutions or suspensions), tablets and capsules, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation.

As used herein, the term “pharmaceutically acceptable carrier” refers to an excipient or diluent in a pharmaceutical composition. For example, a pharmaceutically acceptable carrier may be a vehicle capable of suspending or dissolving the stenoparib or a pharmaceutically acceptable salt thereof or a hydrate thereof. The pharmaceutically acceptable carrier can be compatible with the other ingredients of the formulation and not deleterious to the recipient. For oral administration, a solid carrier may be preferred.

As used herein, the term “pharmaceutically acceptable salt” refers to salt forms (e.g., acid addition salts or metal salts) of stenoparib or dovitinib suitable for therapeutic use according to the methods of the disclosure.

By “pharmaceutical composition” is meant any composition that contains stenoparib or a pharmaceutically acceptable salt thereof or a hydrate thereof or dovitinib or a pharmaceutically acceptable salt thereof or a hydrate thereof combined with a pharmaceutically acceptable carrier that together is suitable for administration to a subject and that treats a cancer or reduces the severity of, or ameliorates, one or more symptoms associated with a cancer. Pharmaceutical compositions useful in the methods of the disclosure can take the form of tablets, gelcaps, capsules, pills, powders, granulates, suspensions, and/or emulsions. In particular instances, pharmaceutical compositions useful in the methods of the disclosure take the form of capsules.

By “biomarker” is meant a nucleic acid molecule (e.g., an mRNA or its complement, for example, a cDNA) or a protein encoded by the nucleic acid molecule present in, or from, a cell or tissue. The expression of the biomarker correlates to the responsiveness (e.g., sensitivity or resistance) of the cell or tissue (and thus, the subject containing the cell or tissue or the subject from which the cell or tissue was obtained) to a cancer treatment (e.g., stenoparib or a pharmaceutically acceptable salt thereof or a hydrate thereof or dovitinib or a pharmaceutically acceptable salt thereof or a hydrate thereof). In particular, a biomarker of sensitivity for the responsiveness to treatment with stenoparib or a pharmaceutically acceptable salt thereof or a hydrate thereof is a nucleic acid molecule (e.g., a mRNA or its complement) expressed from any one of the genes shown in Table 1, or the protein encoded by the nucleic acid molecule, and a biomarker of resistance for the responsiveness to treatment with stenoparib or a pharmaceutically acceptable salt thereof or a hydrate thereof is a nucleic acid molecule (e.g., a mRNA or its complement) expressed from any one of the genes shown in Table 2, or the protein encoded by the nucleic acid molecule. In particular, a biomarker of sensitivity for the responsiveness to treatment with dovitinib or a pharmaceutically acceptable salt thereof or a hydrate thereof is a nucleic acid molecule (e.g., a mRNA or its complement) expressed from any one of the genes shown in Table 3, or the protein encoded by the nucleic acid molecule, and a biomarker of resistance for the responsiveness to treatment with dovitinib or a pharmaceutically acceptable salt thereof or a hydrate thereof is a nucleic acid molecule (e.g., a mRNA or its complement) expressed from any one of the genes shown in Table 4, or the protein encoded by the nucleic acid molecule.

The terms “cancer” and “cancerous” refer to or describe the physiological condition in mammals (e.g., humans) that is typically characterized by unregulated cell proliferation. Examples of cancer include, but are not limited to, myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. The term cancer includes hematological cancers (e.g., cancer of the blood, such as multiple myeloma) and solid tumors (e.g., breast cancer or ovarian cancer). In a particular example, the cancer is an ovarian cancer, a breast cancer, a colon cancer, a pancreatic cancer, or a prostate cancer. In a particular example, the cancer is locally advanced or metastatic. In a particular example, e.g., when the subject is treated with a monotherapy including stenoparib or a pharmaceutically acceptable salt thereof or a hydrate thereof, the cancer is homologous recombination (HR) deficient. In a particular example, e.g., when the subject is treated with a monotherapy including stenoparib or a pharmaceutically acceptable salt thereof or a hydrate thereof, the cancer includes a BRCA mutation. In a particular example, e.g., when the subject is treated with a combination therapy including stenoparib or a pharmaceutically acceptable salt thereof or a hydrate thereof and dovitinib or a pharmaceutically acceptable salt thereof or a hydrate thereof, the cancer is HR proficient. In a particular example, e.g., when the subject is treated with a monotherapy including stenoparib or a pharmaceutically acceptable salt thereof or a hydrate thereof, the cancer is an ovarian cancer, and the ovarian cancer is an epithelial ovarian carcinoma. In some examples, an ovarian cancer may include a high-grade serous epithelial ovarian carcinoma, an endometrioid epithelial ovarian carcinoma, a clear cell epithelial ovarian carcinoma, an epithelial ovarian carcinosarcoma, an undifferentiated epithelial ovarian carcinoma, or an epithelial ovarian carcinoma of mixed histological subtypes.

The term “BRCA mutation” refers to a mutation in the breast cancer type 1 susceptibility protein (BRCA1) or breast cancer type 2 susceptibility protein (BRCA2) gene. A subject or subject having a BRCA mutation is at higher risk of developing a cancer (e.g., a breast cancer or an ovarian cancer). In some instances, BRCA mutations (e.g., particularly in BRCA2) affect a cell's ability to undergo homologous recombination (HR).

The terms “homologous recombination deficient,” “HR deficient,” “homologous recombination deficiency,” “HR deficiency,” and “HRD” refer to deficiencies that occur during homologous recombination (HR), i.e., during meiosis. Deficiencies in homologous recombination is associated with an increased risk of cancer. (e.g., in humans). Decreased rates of homologous recombination may decrease efficiency of DNA repair, which may lead to cancer. In some instances, BRCA mutation may lead to HR deficiency.

The terms “homologous recombination proficient,” “HR proficient,” “homologous recombination proficiency,” and “HR proficiency” refer to proficiency in homologous recombination.

Specifically, as applied to cancers and tumors (e.g., of solid tumor cancers), HR proficient cancer cells are able to repair DNA damage through homologous recombination. In particular, HR proficient cancers are difficult to treat because of this ability to repair DNA damage. In some examples, cancer cells or tumors may become resistant to a PARP1 inhibitor (e.g., stenoparib) if the cancer cells or tumors are or become HR proficient, and gain the ability to repair DNA through homologous recombination.

The terms “expression level” and “level of expression,” as used herein interchangeably, refer to the amount of a gene product in a cell, tissue, biological sample, organism, or subject, e.g., amounts of DNA, RNA (e.g., messenger RNA (mRNA)), or proteins of a given gene.

“Gene” as used herein indicates a coding or noncoding gene whose activity can be determined by measuring the produced RNA. Examples include protein coding genes, microRNAs, small nuclear RNAs and other RNAs with catalytic, regulatory or coding properties.

To “inhibit growth” as used herein means causing a reduction in cell growth (e.g., cancer cell growth, e.g., as compared to the growth inhibition of the NCI60 cancer cell lines as a reference) in vivo or in vitro by, e.g., 10%, 20%, 30%, 40%, 50%, 60%, 70%, 80%, 90%, 95%, or 99% or more, as evident by a reduction in the proliferation of cells exposed to a treatment (e.g., stenoparib or a pharmaceutically acceptable salt thereof or a hydrate thereof or dovitinib or a pharmaceutically acceptable salt thereof or a hydrate thereof), relative to the proliferation of cells in the absence of the treatment. Growth inhibition may be the result of a treatment (e.g., stenoparib or a pharmaceutically acceptable salt thereof or a hydrate thereof or dovitinib or a pharmaceutically acceptable salt thereof or a hydrate thereof) that induces apoptosis in a cell, induces necrosis in a cell, slows cell cycle progression, disrupts cellular metabolism, induces cell lysis, or induces some other mechanism that reduces the proliferation of cells.

“Microarray” as used herein means a device employed by any method that quantifies one or more subject oligonucleotides, e.g., RNA, DNA, cDNA, or analogues thereof, at a time. For example, many DNA microarrays, including those made by Affymetrix (e.g., an Affymetrix HG-U133A array), use several probes for determining the expression of a single gene. The DNA microarray may contain oligonucleotide probes that may be, e.g., full-length cDNAs complementary to an RNA or cDNA fragments that hybridize to part of an RNA. The DNA microarray may also contain modified versions of DNA or RNA, such as locked nucleic acids or LNA. Exemplary RNAs include mRNA, miRNA, and miRNA precursors.

As used herein, the term “percent (%) sequence identity” refers to the percentage of nucleic acid residues of a candidate sequence, e.g., a probe or primer described herein, that are identical to the nucleic acid residues of a reference sequence, e.g., a biomarker sequence described herein, after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using computer software, such as BLAST, BLAST-2, BLAST-P, BLAST-N, BLAST-X, WU-BLAST-2, ALIGN, ALIGN-2, CLUSTAL, Megalign (DNASTAR). In addition, those skilled in the art can determine appropriate parameters for measuring alignment, including any algorithms needed to achieve optimal alignment over the length of the sequences being compared.

“NCI60” as used herein means a panel of 60 cancer cell lines from lung, colon, breast, ovarian, leukemia, renal, melanoma, prostate and brain cancers including the following cancer cell lines: NSCLC_NCIH23, NSCLC_NCIH522, NSCLC_A549ATCC, NSCLC_EKVX, NSCLC_NCIH226, NSCLC_NCIH332M, NSCLC_H460, NSCLC_HOP62, NSCLC_HOP92, COLON_HT29, COLON_HCC-2998, COLON_HCT116, COLON_SW620, COLON_COLO205, COLON_HCT15, COLON_KM12, BREAST_MCF7, BREAST_MCF7ADRr, BREAST_MDAMB231, BREAST_HS578T, BREAST_MDAMB435, BREAST_MDN, BREAST_BT549, BREAST_T47D, OVAR_OVCAR3, OVAR_OVCAR4, OVAR_OVCAR5, OVAR_OVCAR8, OVAR_IGROV1, OVAR_SKOV3, LEUK_CCRFCEM, LEUK_K562, LEUK_MOLT4, LEUK_HL60, LEUK_RPMI8266, LEUK_SR, RENAL_UO31, RENAL_SN12C, RENAL_A498, RENAL_CAKI1, RENAL_RXF393, RENAL_7860, RENAL_ACHN, RENAL_TK10, MELAN_LOXIMVI, MELAN_MALME3M, MELAN_SKMEL2, MELAN_SKMEL5, MELAN_SKMEL28, MELAN_M14, MELAN_UACC62, MELAN_UACC257, PROSTATE_PC3, PROSTATE_DU145, CNS_SNB19, CNS_SNB75, CNS_U251, CNS_SF268, CNS_SF295, and CNS_SF539.

The terms “patient” and “subject,” as used interchangeably herein, refer to any animal (e.g., a mammal, such as a human). A subject to be treated or tested for responsiveness to a treatment (e.g., dovitinib or a pharmaceutically acceptable salt thereof or a hydrate thereof) according to the methods described herein may be one who has been diagnosed with a cancer, such as ovarian cancer or breast cancer. Diagnosis may be performed by any method or techniques known in the art, such as x-ray, MRI, or biopsy, and confirmed by a physician. To minimize exposure of a subject to drug treatments that may not be therapeutic, the subject may be determined to be either responsive or non-responsive to a cancer treatment, such as stenoparib or a pharmaceutically acceptable salt thereof or a hydrate thereof or dovitinib or a pharmaceutically acceptable salt thereof or a hydrate thereof, according to the methods described herein.

“Resistance,” as used herein, refers to the likelihood that a cancer treatment (e.g., stenoparib or a pharmaceutically acceptable salt thereof or a hydrate thereof or dovitinib or a pharmaceutically acceptable salt thereof or a hydrate thereof) does not have (e.g., does not induce) a desired anti-cancer effect (or, alternatively, refers to the inability of a desired effect to be caused or induced by the cancer treatment in a cell (e.g., a cancer cell) or a tissue (e.g., a tumor) in vitro or in vivo (e.g., in a subject with a cancer, such as a human). For example, resistance refers to the likelihood that a cell (e.g., a cancer cell) or a tissue (e.g., a tumor) in vitro or in vivo (e.g., in a subject with a cancer, such as a human) is tolerant to treatment with an anti-cancer agent (e.g., stenoparib or a pharmaceutically acceptable salt thereof or a hydrate thereof or dovitinib or a pharmaceutically acceptable salt thereof or a hydrate thereof), e.g., the cell or tissue is able to survive and grow despite exposure to (e.g., treatment with) an anti-cancer agent (e.g., stenoparib or a pharmaceutically acceptable salt thereof or a hydrate thereof or dovitinib or a pharmaceutically acceptable salt thereof or a hydrate thereof). For example, a resistant cell or tissue is one that, upon exposure of the cell (e.g., a cancer cell) or the tissue (e.g., a tumor), respectively, to a cancer therapeutic (e.g., stenoparib or a pharmaceutically acceptable salt thereof or a hydrate thereof or dovitinib or a pharmaceutically acceptable salt thereof or a hydrate thereof) exhibits an inhibition in growth of the cell or tumor of less than 30%, 25%, 20%, 15%, 10%, 5%, or 1% relative to the growth of a cell or tissue not exposed to the treatment. Resistance to treatment may be determined by a cell proliferation assay, e.g., a cell-based assay, which measures the growth of treated cells as a function of the absorbance of the cells of an incident light beam, such as the NCI60 assays described herein. In this assay, greater absorbance indicates greater cell growth, and thus, resistance to the treatment.

The terms “sensitivity” and “responsiveness,” as used herein, refer to the likelihood that a cancer treatment (e.g., stenoparib or a pharmaceutically acceptable salt thereof or a hydrate thereof or dovitinib or a pharmaceutically acceptable salt thereof or a hydrate thereof) has (e.g., induces) a desired effect, or alternatively refers to the strength of a desired effect caused or induced by the treatment in a cell (e.g., a cancer cell) or a tissue (e.g., a tumor) in vitro or in vivo (e.g., in a subject with a cancer, such as a human). For example, the desired effect can include inhibition of the growth of a cell (e.g., a cancer cell) in vitro by more than 30%, 35%, 40%, 45%, 50%, 55%, 60%, 65%, 70%, 75%, 80%, 85%, 90%, 95%, or 100% relative to the growth of a cell (e.g., a cancer cell) not exposed to the treatment. The desired effect can also include reduction in tumor mass by, e.g., about 30%, 35%, 40%, 45%, 50%, 55%, 60%, 65%, 70%, 75%, 80%, 85%, 90%, 95%, or 100%. “Sensitive” and “responsive” as used herein refer to a cell (e.g., a cancer cell) or a tissue (e.g., a tumor) in vitro or in vivo (e.g., in a subject with a cancer, such as a human) that is responsive to exposure to a therapeutic (e.g., stenoparib or a pharmaceutically acceptable salt thereof or a hydrate thereof or dovitinib or a pharmaceutically acceptable salt thereof or a hydrate thereof). Responsiveness to treatment may be determined by a cell proliferation assay, e.g., a cell-based assay, which measures the growth of treated cells as a function of the absorbance of the cells of an incident light beam, such as the NCI60 assays described herein. In this assay, lesser absorbance indicates lesser cell growth, and thus, sensitivity or responsiveness to the treatment. A greater reduction in growth indicates more sensitivity or responsiveness to the treatment. In some instances, a response exhibited by a subject after treatment with any of the methods described herein may be determined using the Response Evaluation Criteria in Solid Tumors (RECIST) 1.1. In some instances, a response exhibited by a subject after treatment with any of the methods described herein may be a complete response (CR), a partial response (PR), stable disease (SD), or SD for at least 16 weeks.

The term “sample,” as used herein, refers to any specimen (such as cells, tissue (e.g., a tissue sample obtained by biopsy), blood, serum, plasma, urine, cerebrospinal fluid, or pancreatic fluid) taken from a subject. Preferably, the sample is taken from a portion of the body affected by a cancer (e.g., a biopsy of the cancer tissue). Biopsy may involve fine needle aspiration biopsy, core needle biopsy (e.g., stereotactic core needle biopsy, vacuum-assisted core biopsy, or magnetic resonance imaging (MRI) guided biopsy), or surgical biopsy (e.g., incisional biopsy or excisional biopsy). The sample may undergo additional purification and processing, for example, to remove cell debris and other unwanted molecules. Additional processing may further involve amplification, e.g., using PCR (e.g., RT-PCR). The standard methods of sample purification, such as removal of unwanted molecules, are known in the art.

“Substantially similar” or “corresponds,” as used herein with respect to a numerical value of a parameter of one or more of the biomarker(s) of sensitivity and/or resistance (e.g., biomarker expression level, difference score, or mean score), e.g., as determined in a test sample (e.g., a tumor biopsy) from a cancer subject, means that the numerical value of the parameter in the test sample is +0-30% of the numerical value of the parameter in a reference sample (e.g., a cell (e.g., a cancer cell) or tissue (e.g., a tumor) known to be sensitive or resistant to stenoparib or a pharmaceutically acceptable salt thereof or a hydrate thereof or dovitinib or a pharmaceutically acceptable salt thereof or a hydrate thereof). For example, a numerical value of a parameter in a test sample may be substantially similar to, or may correspond to, the numerical value of the parameter in a reference sample if the parameter values of the test and reference samples differ by, e.g., less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1%.

“Substantially dissimilar,” as used herein with respect to a numerical value of a parameter of one or more of the biomarker(s) of sensitivity and/or resistance (e.g., biomarker expression level, difference score, or mean score), e.g., as determined in a test sample (e.g., a tumor biopsy) from a cancer subject, means that the numerical value of the parameter in the test sample deviates by greater than 30% from the numerical value of the parameter in a reference sample (e.g., a cell (e.g., a cancer cell) or tissue (e.g., a tumor) known to be sensitive or resistant to stenoparib or a pharmaceutically acceptable salt thereof or a hydrate thereof or dovitinib or a pharmaceutically acceptable salt thereof or a hydrate thereof). For example, a numerical value of a parameter in a test sample may be substantially dissimilar to the numerical value of the parameter in a reference sample if the parameter values of the test and reference samples differ by, e.g., greater than 35%, 40%, 45%, 50%, 55%, 60%, 65%, 70%, 75%, 80%, 85%, 90%, 95%, 100% or more.

“Treatment,” “medical treatment,” to “treat,” and “therapy,” as used interchangeably herein, refer to administering or exposing a subject with a cancer (e.g., a human), a cancer cell, or a tumor to an anti-cancer agent (e.g., a drug, a protein, an antibody, a nucleic acid, a chemotherapeutic agent, or a radioactive agent; e.g., stenoparib or a pharmaceutically acceptable salt thereof or a hydrate thereof or dovitinib or a pharmaceutically acceptable salt thereof or a hydrate thereof), or to some other form of medical intervention used to treat or prevent a disease, disorder, or condition (e.g., surgery, cryotherapy, radiation therapy, or combinations thereof). In particular, a medical treatment can include stenoparib or a pharmaceutically acceptable salt thereof or a hydrate thereof or dovitinib or a pharmaceutically acceptable salt thereof or a hydrate thereof. For example, the cancer to be treated is a hematological cancer or a solid tumor. Examples of cancer include, e.g., myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, endometrial cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma, and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, or neoplasms of the central nervous system. Radiation therapy includes the administration of a radioactive agent to a subject or exposure of a subject to radiation. The radiation may be generated from sources such as particle accelerators and related medical devices or agents that emit, e.g., X-radiation, gamma radiation, or electron (Beta radiation) beams. A treatment may be or further include surgery, e.g., to remove a tumor from a subject or living organism.

A “CA125 test” as used herein refers to a test for determining the amount of protein CA125 (cancer antigen 125) in the blood. In some instances, this test may be used to monitor a cancer in a subject during and/or after the subject receives a treatment. In some instances, a CA125 test may be used to monitor the status of an ovarian cancer in a subject. CA125 may also be known as MUC-16.

Other features and advantages of the invention will be apparent from the following Detailed Description, the drawings, and the claims.

### BRIEF DESCRIPTION OF THE DRAWINGS

FIG. 1A and FIG. 1B are graphs showing the pharmacokinetics of stenoparib administration. The different lines represent different doses of stenoparib (E7449) administered at time 0. Concentration of stenoparib in blood plasma is shown in ng/ml. In comparing Day-2 administration (FIG. 1A) and Day 15 administration (FIG. 1B), minimal accumulation based on AUC was observed.

FIG. 2A and FIG. 2B are graphs showing the pharmacokinetics of stenoparib administration after food intake. The different lines represent 600 mg doses of stenoparib (E7449) administered at time 0 with or without food intake. Concentration of stenoparib in blood plasma is shown in ng/ml. Food intake vs fasting comparisons were made after administration of the Cycle 1 Day 7 dose (FIG. 2A) and after the Cycle 1 Day 15 dose (FIG. 2B).

FIG. 3 is a diagram showing a 3+3 dose escalation study design for determining the maximum tolerated dose (MTD), e.g., of stenoparib or of dovitinib.

FIG. 4A and FIG. 4B are waterfall plots showing the best change in tumor diameter from baseline in the IAP QD population (FIG. 4A) and the IAP BID population (FIG. 4B). CR: complete response; SD: stable disease; PD: disease progression. Numerical values below each bar report the % change value of the corresponding bar. The CR is unconfirmed, and consists of target lymph node lesions that all turn normal. This CR patient also has CA125 CR unconfirmed.

FIG. 5 is a Kaplan-Meier plot of progression-free survival in the IAP population stratified by stenoparib dosing: QD vs BID. Censored patients, for whom a date of progression or death has not yet been established, are indicated with a vertical line on each curve.

FIG. 6A and FIG. 6B are stripcharts of DRP scores in the individual RECIST categories. FIG. 6A shows a stripchart of DRP scores in individual response categories in QD dosed patients. FIG. 6B. shows a stripchart of DRP scores in individual response categories in BID dosed patients.

### DETAILED DESCRIPTION

The disclosure features methods for treating a cancer, such as a solid tumor cancer, including, for example, ovarian cancer or breast cancer. The methods include administering stenoparib or a pharmaceutically acceptable salt thereof or a hydrate thereof (e.g., twice a day (i.e., BID dosing)) and/or dovitinib or a pharmaceutically acceptable salt thereof or a hydrate thereof (e.g., once a day (i.e., QD dosing) to a subject having a cancer.

The disclosure features twice-a-day dosing (BID dosing) of stenoparib for the treatment of cancer (e.g., solid tumor cancer, such as prostate cancer, breast cancer, and ovarian cancer), which, unexpectedly, produces improved therapeutic outcomes. The inventors observed that subjects with ovarian cancer who were treated with once-a-day dosing of stenoparib did not exhibit an expected therapeutic benefit that was predicted from in vitro studies and in vivo animal studies. In particular, it was discovered that, in human subjects, the half-life of stenoparib was unexpectedly only about 8 hours, which was insufficient to impact the biological and pathological features of the cancer or to improve the survival of the treated subjects. By contrast, twice-a-day dosing of stenoparib increases the length of time a subject is exposed to pharmacokinetically effective concentrations of stenoparib, thereby resulting in improved therapeutic outcomes.

Stenoparib or a pharmaceutically acceptable salt thereof or a hydrate thereof may be administered alone or in combination with dovitinib or a pharmaceutically acceptable salt thereof or a hydrate thereof, and optionally, with another therapeutic agent, in order to treat a cancer in a subject.

In some instances, a subject being treated with a method described herein may have a hematological cancer or a solid tumor, such as a cancer type selected from myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. In particular, the cancer of the subject is, e.g., multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, estrogen receptor-positive (ERpos) breast cancer, lung cancer, non-small cell lung carcinoma (NSCLC), mesothelioma, intestinal cancer, colon cancer, bladder cancer, adrenal cancer, gallbladder cancer, and squamous cell carcinoma of the head and neck (SCCHN). The subject may have estrogen receptor-positive (ER pos) breast cancer. The subject may also have a locally advanced or metastatic form of a cancer. The subject may also have recurrence of cancer, such as a recurrent form of any of the above cancer types, e.g., recurrent breast cancer, recurrent ovarian cancer, or recurrent pancreatic cancer. In a particular example, the cancer is a solid tumor cancer. In a particular example, the cancer is an ovarian cancer, a breast cancer, a colon cancer, a pancreatic cancer, or a prostate cancer. In a particular example, the cancer is locally advanced or metastatic. In a particular example, e.g., when the subject is treated with a monotherapy including stenoparib or a pharmaceutically acceptable salt thereof or a hydrate thereof, the cancer is homologous recombination (HR) deficient. In a particular example, e.g., when the subject is treated with a monotherapy including stenoparib or a pharmaceutically acceptable salt thereof or a hydrate thereof, the cancer includes a BRCA mutation. In a particular example, the ovarian cancer is an epithelial ovarian carcinoma. In some examples, an ovarian cancer is a high-grade serous, endometrioid, clear cell, carcinosarcoma, or undifferentiated ovarian cancer, or an ovarian cancer of mixed histological subtypes. In some examples, an ovarian cancer is a high-grade serous, endometrioid, clear cell, carcinosarcoma, or undifferentiated ovarian cancer, or an ovarian cancer of mixed histological subtypes independent of BRCA1 and HRD status. In a particular example, the ovarian cancer is advanced, refractory, or recurrent ovarian cancer. In a particular example, the ovarian cancer is refractory to or has recurred after treatment with two or more previous chemotherapies and/or antibody therapies. In a particular example, the previous chemotherapies and/or antibody therapies comprise another Poly (ADP-ribose) Polymerase (PARP) inhibitor (PARPi). In a particular example, e.g., when the subject is treated with a combination therapy including stenoparib or a pharmaceutically acceptable salt thereof or a hydrate thereof and dovitinib or a pharmaceutically acceptable salt thereof or a hydrate thereof, the cancer is HR proficient.

### Administration of Stenoparib Monotherapy

The disclosure features methods for treating a subject having a cancer by administering stenoparib monotherapy. In particular, the disclosure features a method of treating a cancer in a subject in need thereof including administering to the subject a dosing regimen including a first dose and a second dose of a composition including stenoparib or a pharmaceutically acceptable salt thereof or a hydrate thereof per day.

The dosing regimen, e.g., for stenoparib monotherapy, includes a dosing cycle including administration of the composition for 28 days±1 to 5 days (e.g., 23 days, 24 days, 25 days, 26 days, 27 days, 28 days, 29 days, 30 days, 31 days, 32 days, or 33 days). In some instances, the dosing cycle may be 21 days. In a particular instance, the dosing cycle may be 28 days in length. Stenoparib or a pharmaceutically acceptable salt thereof or a hydrate thereof may be administered daily, including a first dose and a second dose, e.g., as a twice-a-day or BID dosing. The dosing regimen may include more than one dosing cycle. For example, the dosing regimen may include 2 to 100 dosing cycles (e.g., 2 to 3, 2 to 4, 2 to 5, 2 to 6, 2 to 7, 2 to 8, 2 to 9, 2 to 10, 2 to 15, 2 to 20, 2 to 25, 2 to 30, 2 to 40, 2 to 50, 2 to 60, 2 to 70, 2 to 80, 2 to 90, 5 to 10, 10 to 20, 20 to 30, 30 to 50, 50 to 70, or 70 to 100 dosing cycles; e.g., 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 dosing cycles). In a particular example, the dosing regimen includes 2 to 8 dosing cycles (e.g., 2, 3, 4, 5, 6, 7, or 8 dosing cycles).

In some instances, an amount of stenoparib or the pharmaceutically acceptable salt thereof or the hydrate thereof administered in the first dose is less than or equal to an amount of stenoparib or the pharmaceutically acceptable salt thereof or the hydrate thereof administered in the second dose. In some instances, the first dose includes between about 200 mg to about 400 mg (e.g., about 200 mg to about 250 mg, about 250 mg to about 300 mg, about 350 mg to about 400 mg, about 200 mg to about 300 mg, or about 300 mg to about 400 mg; e.g., about 200 mg, about 250 mg, about 300 mg, about 350 mg, or about 400 mg) of stenoparib or the pharmaceutically acceptable salt thereof or the hydrate thereof. In some instances, the second dose includes between about 400 mg to about 600 mg (e.g., about 400 mg to about 450 mg, about 450 mg to about 500 mg, about 550 mg to about 600 mg, about 400 mg to about 500 mg, or about 500 mg to about 600 mg; e.g., about 400 mg, about 450 mg, about 500 mg, about 550 mg, or about 600 mg) of stenoparib or the pharmaceutically acceptable salt thereof or the hydrate thereof. In one particular example, the first dose includes about 200 mg of stenoparib or the pharmaceutically acceptable salt thereof or the hydrate thereof and the second dose includes about 400 mg of stenoparib or the pharmaceutically acceptable salt thereof or the hydrate thereof. In another particular example, the first dose includes about 400 mg of stenoparib or the pharmaceutically acceptable salt thereof or the hydrate thereof and the second dose includes about 400 mg of stenoparib or the pharmaceutically acceptable salt thereof or the hydrate thereof. In yet another particular example, the first dose includes about 400 mg of stenoparib or the pharmaceutically acceptable salt thereof or the hydrate thereof and the second dose includes about 600 mg of stenoparib or the pharmaceutically acceptable salt thereof or the hydrate thereof.

In some instances, the second dose is administered to the subject at least 11 to 13 hours (e.g., at least 11, at least 11.5, at least 12, at least 12.5, or at least 13 hours) after administration of the first dose. In some instances, the second dose is administered to the subject at least 6, 7, 8, 9, 10, 11, 12, 13, 14, 15, 16, 17 or 18 hours after administration of the first dose.

In some instances, the first dose and the second dose of the composition is administered to the subject at about the same time during each day of a dosing cycle. For example, if stenoparib or a pharmaceutically acceptable salt thereof or a hydrate thereof is administered to a subject as a first dose at about 8 am and as a second dose at about 8 pm on Day 1 of a first dosing cycle, then stenoparib or the pharmaceutically acceptable salt thereof or the hydrate thereof is administered as a first dose at about 8 am and as a second dose at about 8 pm on Days 2-28 of the first dosing cycle, and optionally also as a first dose at about 8 am and as a second dose at about 8 pm on Days 1-28 of any additional dosing cycles.

In some instances, the subject being administered stenoparib monotherapy is non-responsive to at least one cancer therapy (e.g., one, two, three, four, five, six, seven, eight, nine, ten, or more different cancer therapies) other than stenoparib or a pharmaceutically acceptable salt thereof or a hydrate thereof. In a particular instance, the cancer therapy a PARP inhibitor (PARPi) other than stenoparib.

In some instances, the method includes, prior to administration of the composition, determining the responsiveness of the subject to treatment with stenoparib or the pharmaceutically acceptable salt thereof or the hydrate thereof by determining the expression level of one or more (e.g., five, ten, fifteen, or twenty or more) biomarkers of sensitivity selected from the biomarkers of Table 1; and/or determining the expression level of one or more (e.g., five, ten, fifteen, or twenty or more) biomarkers of resistance selected from the biomarkers of Table 2. Methods of determining the responsiveness of a subject to treatment with stenoparib or a pharmaceutically acceptable salt thereof or a hydrate thereof are described in detail below. In addition, see the methods for determining sensitivity and resistance to stenoparib described in PCT Patent Publication No. WO 2019/219759A1 and U.S. Patent Publication No. US 20210/222252A1, each of which is incorporated by reference in its entirety.

In some instances, a subject treated with stenoparib monotherapy according to any of the methods disclosed herein exhibits a complete response (CR). In some instances, a subject treated with stenoparib monotherapy according to any of the methods disclosed herein exhibits a partial response (PR). In some instances, a subject treated with stenoparib monotherapy according to any of the methods disclosed herein exhibits stable disease (SD). In some instances, a subject treated with stenoparib monotherapy according to any of the methods disclosed herein exhibits SD for at least 16 weeks.

In the methods described herein, stenoparib or a pharmaceutically acceptable salt thereof or a hydrate thereof is administered twice a day (i.e., BID dosing).

The route for administration of stenoparib or a pharmaceutically acceptable salt thereof or a hydrate thereof may be oral. Stenoparib or a pharmaceutically acceptable salt thereof or a hydrate thereof may be administered as a pharmaceutical composition (e.g., including stenoparib or a pharmaceutically acceptable salt thereof or a hydrate thereof in combination with a pharmaceutically acceptable carrier and/or excipient). The pharmaceutical composition may be formulated as a capsule (e.g., a hard gel capsule) or a tablet (e.g., a film coated tablet). Each capsule or tablet of stenoparib or a pharmaceutically acceptable salt thereof or a hydrate thereof may contain about 50 mg, 100 mg, 150 mg, 200 mg, 250 mg, 300 mg, 350 mg, 400 mg, 450 mg, 500 mg, 550 mg, or 600 mg of stenoparib or the pharmaceutically acceptable salt thereof or the hydrate thereof. In a preferred example, each capsule of stenoparib or the pharmaceutically acceptable salt thereof or the hydrate thereof may contain about 50 mg (e.g., 50±5 mg), about 100 mg (e.g., 100±10 mg), about 150 mg (e.g., 150±15 mg), or about 200 mg (e.g., 200±20 mg) of stenoparib or pharmaceutically acceptable salt thereof.

As an example, when stenoparib is formulated in a tablet or capsule in an amount of about 100 mg (e.g., 100 mg±10 mg), administration of a first dose of about 200 mg (e.g., 200 mg±20 mg) or about 400 mg (e.g., 400 mg±40 mg) of stenoparib involves administering (e.g., orally administering) 2 or 4 of the tablets or capsules comprising stenoparib or a pharmaceutically acceptable salt thereof or a hydrate thereof, respectively. Similarly, administration of the second daily dose of stenoparib of about 400 mg (e.g., 400 mg±40 mg) or 600 mg (e.g., 600 mg±60 mg) involves administering (e.g., orally administering) 4 or 6 of the tablets or capsules comprising stenoparib or a pharmaceutically acceptable salt thereof or a hydrate thereof, respectively. As an alternative example, when stenoparib is formulated in a tablet or capsule in an amount of about 200 mg (e.g., 200 mg±10 mg), administration of a first dose of about 200 mg (e.g., 200 mg±20 mg) or about 400 mg (e.g., 400 mg±40 mg) of stenoparib involves administering (e.g., orally administering) 1 or 2 of the tablets or capsules comprising stenoparib or a pharmaceutically acceptable salt thereof or a hydrate thereof, respectively. Similarly, administration of the second daily dose of stenoparib of about 400 mg (e.g., 400 mg±40 mg) or 600 mg (e.g., 600 mg±60 mg) involves administering (e.g., orally administering) 2 or 3 of the tablets or capsules comprising stenoparib or a pharmaceutically acceptable salt thereof or a hydrate thereof, respectively.

The tablets or capsules can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

### Administration of Stenoparib and Dovitinib Combination Therapy

The disclosure features methods for treating a subject having a cancer by administering a combination therapy including stenoparib, or a pharmaceutically acceptable salt thereof, and dovitinib, or a pharmaceutically acceptable salt thereof. In particular, the disclosure features a method of treating a cancer in a subject in need thereof by administering to the subject a dosing regimen that includes a first dose and a second dose of a first composition including stenoparib or a pharmaceutically acceptable salt thereof or a hydrate thereof per day (e.g., BID dosing); and administering a dose of a second composition including dovitinib or a pharmaceutically acceptable salt thereof or a hydrate thereof.

The combination of stenoparib and dovitinib provides effective anti-tumor therapy (e.g., in treating solid tumors, such as ovarian and breast cancers). Stenoparib is a small molecule, dual-targeted inhibitor of Poly ADP-Ribose Polymerases (PARP 1 and 2) and tankyrase 1 and 2, and functions by limiting the ability of cancer cells within tumors to repair single stranded DNA breaks, which in turn makes the cancer cells more vulnerable to death (apoptosis). Dovitinib is a pan-tyrosine kinase inhibitor (pan-TKI), and its mechanisms of action (MOA) works in part to block the formation of new blood vessels that supply a tumor with nutrients and oxygen (anti-angiogenesis). As these combined MOAs serve to promote cancer cell vulnerability and restrict blood flow necessary to fuel cancer growth and repair, the combination of stenoparib and dovitinib may cause synthetic lethality, thereby increasing the chances of cancer cell death and providing synergistic, enhanced anti-tumor activity. Furthermore, administration of dovitinib may change a homologous recombination (HR)-proficient tumor into an HR-deficient tumor via down-regulation of HR, thus rendering the tumor more susceptible to treatment with stenoparib.

The dosing regimen, e.g., for a stenoparib and dovitinib combination therapy, includes a dosing cycle of 28 days±1 to 5 days (e.g., 23 days, 24 days, 25 days, 26 days, 27 days, 28 days, 29 days, 30 days, 31 days, 32 days, or 33 days). In some instances, the dosing cycle may be 21 days. In a particular instance, the dosing cycle may be 28 days in length. The dosing regimen may include more than one dosing cycle. For example, the dosing regimen may include 2 to 100 dosing cycles (e.g., 2 to 3, 2 to 4, 2 to 5, 2 to 6, 2 to 7, 2 to 8, 2 to 9, 2 to 10, 2 to 15, 2 to 20, 2 to 25, 2 to 30, 2 to 40, 2 to 50, 2 to 60, 2 to 70, 2 to 80, 2 to 90, 5 to 10, 10 to 20, 20 to 30, 30 to 50, 50 to 70, or 70 to 100 dosing cycles; e.g., 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 dosing cycles).

In some instances, the method, e.g., for a stenoparib and dovitinib combination therapy, includes administering first and second doses of the first composition (containing stenoparib or a pharmaceutically acceptable salt thereof or a hydrate thereof) each day of the dosing cycle and administering the second composition (containing dovitinib or a pharmaceutically acceptable salt thereof or a hydrate thereof) on a 5 days on and 2 days off schedule during the dosing cycle. In a particular example, the second composition is administered to the subject as a single dose on Days 1-5, Days 8-12, Days 15-19, and Days 22-26 of each said dosing cycle (e.g., of each said 28-day dosing cycle).

In some instances, an amount of stenoparib or the pharmaceutically acceptable salt thereof or the hydrate thereof administered in the first dose of the first composition is less than or equal to an amount of stenoparib or the pharmaceutically acceptable salt thereof or the hydrate thereof administered in the second dose of the first composition. In some instances, the first dose includes between about 200 mg to about 400 mg (e.g., about 200 mg to about 250 mg, about 250 mg to about 300 mg, about 350 mg to about 400 mg, about 200 mg to about 300 mg, or about 300 mg to about 400 mg; e.g., about 200 mg, about 250 mg, about 300 mg, about 350 mg, or about 400 mg) of stenoparib or the pharmaceutically acceptable salt thereof or the hydrate thereof. In some instances, the second dose includes between about 400 mg to about 600 mg (e.g., about 400 mg to about 450 mg, about 450 mg to about 500 mg, about 550 mg to about 600 mg, about 400 mg to about 500 mg, or about 500 mg to about 600 mg; e.g., about 400 mg, about 450 mg, about 500 mg, about 550 mg, or about 600 mg) of stenoparib or the pharmaceutically acceptable salt thereof or the hydrate thereof.

In a particular example, the first dose of the first composition includes about 200 mg of stenoparib or the pharmaceutically acceptable salt thereof or the hydrate thereof and the second dose of the first composition includes about 400 mg of stenoparib or the pharmaceutically acceptable salt thereof or the hydrate thereof.

In another particular example, the first dose of the first composition includes about 400 mg of stenoparib or the pharmaceutically acceptable salt thereof or the hydrate thereof and the second dose of the first composition includes about 400 mg of stenoparib or the pharmaceutically acceptable salt thereof or the hydrate thereof.

In yet another particular example, the first dose of the first composition includes about 400 mg of stenoparib or the pharmaceutically acceptable salt thereof or the hydrate thereof and the second dose of the first composition includes about 600 mg of stenoparib or the pharmaceutically acceptable salt thereof or the hydrate thereof.

In some instances, the dose of the second composition includes between about 300 mg to about 500 mg (e.g., about 300 mg to about 350 mg, about 350 mg to about 400 mg, about 450 mg to about 500 mg, about 300 mg to about 400 mg, or about 400 mg to about 500 mg; e.g., about 300 mg, about 350 mg, about 400 mg, about 450 mg, or about 500 mg) of dovitinib or the pharmaceutically acceptable salt thereof or the hydrate thereof. In a particular example, the dose of the second composition includes about 300 mg of dovitinib or the pharmaceutically acceptable salt thereof or the hydrate thereof. In another particular example, the dose of the second composition includes about 400 mg of dovitinib or the pharmaceutically acceptable salt thereof or the hydrate thereof. In yet another particular example, the dose of the second composition includes about 500 mg of dovitinib or the pharmaceutically acceptable salt thereof or the hydrate thereof.

In some instances, for each daily administration, the second dose of the first composition is administered to the subject at least 11 to 13 hours (e.g., at least 11, at least 11.5, at least 12, at least 12.5, or at least 13 hours) after administration of the first dose of the first composition. In some instances, the second dose of the first composition is administered to the subject at least 6, 7, 8, 9, 10, 11, 12, 13, 14, 15, 16, 17 or 18 hours after administration of the first dose of the first composition.

In some instances, the first dose and the second dose of the first composition is administered to the subject at about the same time during each day of a dosing cycle. For example, if stenoparib or a pharmaceutically acceptable salt thereof or a hydrate thereof is administered to a subject as a first dose at about 8 am and as a second dose at about 8 pm on Day 1 of a first dosing cycle, then stenoparib or the pharmaceutically acceptable salt thereof or the hydrate thereof is administered as a first dose at about 8 am and as a second dose at about 8 pm on Days 2-28 of the first dosing cycle, and optionally also as a first dose at about 8 am and as a second dose at about 8 pm on Days 1-28 of any additional dosing cycles.

In some instances, the subject is non-responsive to at least one cancer therapy (e.g., one, two, three, four, five, six, seven, eight, nine, ten, or more cancer therapies) other than stenoparib or a pharmaceutically acceptable salt thereof or a hydrate thereof and/or dovitinib and or a pharmaceutically acceptable salt thereof. In a particular instance, the cancer therapy is a PARP inhibitor (PARPi) other than stenoparib.

In some instances, the method includes, prior to administration of the first composition, determining the responsiveness of the subject to treatment with stenoparib or the pharmaceutically acceptable salt thereof or the hydrate thereof by determining the expression level of one or more biomarkers of sensitivity selected from the biomarkers of Table 1; and/or determining the expression level of one or more biomarkers of resistance selected from the biomarkers of Table 2. Methods of determining the responsiveness of a subject to treatment with stenoparib or a pharmaceutically acceptable salt thereof or a hydrate thereof are described in detail below.

In some instances, the method includes, prior to administration of the second composition, determining the responsiveness of the subject to treatment with dovitinib or the pharmaceutically acceptable salt thereof or the hydrate thereof by determining the expression level of one or more biomarkers of sensitivity selected from the biomarkers of Table 3; and/or determining the expression level of one or more biomarkers of resistance selected from the biomarkers of Table 4. Methods of determining the responsiveness of a subject to treatment with dovitinib or a pharmaceutically acceptable salt thereof or a hydrate thereof are described in detail below.

In some instances, a subject treated with stenoparib and dovitinib combination therapy according to any of the methods disclosed herein exhibits a complete response (CR). In some instances, a subject treated with stenoparib and dovitinib combination therapy according to any of the methods disclosed herein exhibits a partial response (PR). In some instances, a subject treated with stenoparib and dovitinib combination therapy according to any of the methods disclosed herein exhibits stable disease (SD). In some instances, a subject treated with stenoparib and dovitinib combination therapy according to any of the methods disclosed herein exhibits SD for at least 16 weeks.

In the methods described herein, stenoparib or a pharmaceutically acceptable salt thereof or a hydrate thereof is administered twice a day (e.g., BID dosing).

The route for administration of stenoparib or a pharmaceutically acceptable salt thereof or a hydrate thereof may be oral. Stenoparib or a pharmaceutically acceptable salt thereof or a hydrate thereof may be administered as a pharmaceutical composition (e.g., including stenoparib or a pharmaceutically acceptable salt thereof or a hydrate thereof in combination with a pharmaceutically acceptable carrier and/or excipient). The pharmaceutical composition (e.g., the first composition) may be formulated as a capsule (e.g., a hard gel capsule) or a tablet (e.g., a film coated tablet). Each capsule or tablet of stenoparib or a pharmaceutically acceptable salt thereof or a hydrate thereof may contain about 50 mg, 100 mg, 150 mg, 200 mg, 250 mg, 300 mg, 350 mg, 400 mg, 450 mg, 500 mg, 550 mg, or 600 mg of stenoparib or the pharmaceutically acceptable salt thereof or the hydrate thereof. In an example, each capsule of stenoparib or the pharmaceutically acceptable salt thereof or the hydrate thereof may contain about 50 mg (e.g., 50 mg±5 mg), about 100 mg (e.g., 100 mg±10 mg), about 150 mg (e.g., 150 mg±15 mg), or about 200 mg (e.g., 200 mg±20 mg) of stenoparib or pharmaceutically acceptable salt thereof.

As an example, when stenoparib is formulated in a tablet or capsule in an amount of about 100 mg (e.g., 100 mg±10 mg), administration of a first dose of about 200 mg (e.g., 200 mg±20 mg) or about 400 mg (e.g., 400 mg±40 mg) of stenoparib involves administering (e.g., orally administering) 2 or 4 of the tablets or capsules comprising stenoparib or a pharmaceutically acceptable salt thereof or a hydrate thereof, respectively. Similarly, administration of the second daily dose of stenoparib of about 400 mg (e.g., 400 mg±40 mg) or 600 mg (e.g., 600 mg±60 mg) involves administering (e.g., orally administering) 4 or 6 of the tablets or capsules comprising stenoparib or a pharmaceutically acceptable salt thereof or a hydrate thereof, respectively. As an alternative example, when stenoparib is formulated in a tablet or capsule in an amount of about 200 mg (e.g., 200 mg±10 mg), administration of a first dose of about 200 mg (e.g., 200 mg±20 mg) or about 400 mg (e.g., 400 mg±40 mg) of stenoparib involves administering (e.g., orally administering) 1 or 2 of the tablets or capsules comprising stenoparib or a pharmaceutically acceptable salt thereof or a hydrate thereof, respectively. Similarly, administration of the second daily dose of stenoparib of about 400 mg (e.g., 400 mg±40 mg) or 600 mg (e.g., 600 mg±60 mg) involves administering (e.g., orally administering) 2 or 3 of the tablets or capsules comprising stenoparib or a pharmaceutically acceptable salt thereof or a hydrate thereof, respectively.

In the methods described herein, dovitinib is administered once a day (e.g., QD dosing).

The route for administration of dovitinib or a pharmaceutically acceptable salt thereof or a hydrate thereof may be oral. Dovitinib or a pharmaceutically acceptable salt thereof or a hydrate thereof may be administered as a pharmaceutical composition (e.g., including dovitinib or a pharmaceutically acceptable salt thereof or a hydrate thereof in combination with a pharmaceutically acceptable carrier and/or excipient). The pharmaceutical composition (e.g., the second composition) may be formulated as a tablet (e.g., a film coated tablet). Each tablet of dovitinib or a pharmaceutically acceptable salt thereof or a hydrate thereof may contain about 50 mg, 100 mg, 150 mg, 200 mg, 250 mg, 300 mg, 350 mg, 400 mg, 450 mg, or 500 mg of dovitinib or the pharmaceutically acceptable salt thereof or the hydrate thereof. In an example, each tablet of dovitinib or the pharmaceutically acceptable salt thereof or the hydrate thereof may contain about 100 mg (e.g., 100±10 mg) of dovitinib.

As an example, when dovitinib is formulated in a tablet or capsule in an amount of about 100 mg (e.g., 100 mg±10 mg), administration of a single dose of about 300 mg (e.g., 300 mg±30 mg), about 400 mg (e.g., 400 mg±40 mg), or about 500 mg (e.g., 500 mg±50 mg) of dovitinib involves administering (e.g., orally administering) 3, 4, or 5 of the tablets or capsules comprising dovitinib or a pharmaceutically acceptable salt thereof or a hydrate thereof, respectively.

The tablets or capsules can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In some instances, for combination therapy of stenoparib and dovitinib, on the days when stenoparib and dovitinib are both administered, dovitinib is administered prior to (e.g., about 0.5, 1, 2, 3, 4, 5, 6, 7, 8, 9, 10, 11, 12, 14, 16, 18, 20, 22, or 24 hours prior to) administration of the first dose of stenoparib. In some instances, for combination therapy of stenoparib and dovitinib, on the days when stenoparib and dovitinib are both administered, dovitinib is administered after administration of the first dose of stenoparib. In a preferred example, on the days when stenoparib and dovitinib are both administered, dovitinib is administered prior to (e.g., about 0.5, 1, 2, 3, 4, 5, 6, 7, 8, 9, 10, 11, 12, 14, 16, 18, 20, 22, or 24 hours prior to) administration of the first dose of stenoparib.

### Pharmaceutical Compositions

The methods described herein include administration of stenoparib and a pharmaceutically acceptable salt as a monotherapy or administration of stenoparib and a pharmaceutically acceptable salt and dovitinib and a pharmaceutically acceptable salt as a combination therapy (e.g., both agents are administered separately to the subject). Pharmaceutically acceptable compositions including stenoparib and dovitinib, as well as pharmaceutically acceptable salts thereof, are described below.

### Stenoparib and Pharmaceutically Acceptable Salts

A subject as described herein may be treated for a cancer by administration of stenoparib or a pharmaceutically acceptable salt of stenoparib. Stenoparib or a pharmaceutically acceptable salt thereof or a hydrate thereof may be administered in a dosage form. Pharmaceutically acceptable salts of stenoparib described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of stenoparib described herein or separately by reacting a free base group with a suitable organic acid.

Stenoparib may have ionizable groups so as to be capable of preparation as a pharmaceutically acceptable salt. These salts may be acid addition salts involving inorganic or organic acids or the salts may, in the case of acidic forms of stenoparib be prepared from inorganic or organic bases. Frequently, stenoparib may be prepared or used as a pharmaceutically acceptable salt prepared as an addition product of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like. In a particular example, the salt form of stenoparib is stenoparib L-tartrate salt.

Stenoparib or a pharmaceutically acceptable salt thereof or a hydrate thereof can be administered in a pharmaceutical composition that includes one or more pharmaceutically acceptable carriers, excipients, or diluents. Examples of suitable carriers, excipients, or diluents of stenoparib or a pharmaceutically acceptable salt thereof or a hydrate thereof are described in the Handbook of Pharmaceutical Excipients, 6th Edition, Rowe et al., Eds., Pharmaceutical Press (2009), hereby incorporated by reference in its entirety.

A pharmaceutical composition can be formulated to be compatible with its intended route of administration. Oral compositions can include an inert diluent or an edible carrier. The composition can be enclosed in a gelatin capsule or compressed into a tablet. For the purpose of oral therapeutic administration, stenoparib or a pharmaceutically acceptable salt thereof or a hydrate thereof can be incorporated with excipients and used in the form of tablets, troches, or gelatin capsules. In particular, stenoparib or a pharmaceutically acceptable salt thereof or a hydrate thereof can be formulated as hard gelatin capsules (e.g., hard gelatin capsules of 10 mg, 50 mg, or 200 mg) or film coated tablets (e.g., film coated tablets of 10 mg, 50 mg, or 200 mg). Preferably, stenoparib or a pharmaceutically acceptable salt thereof or a hydrate thereof can be formulated as hard gelatin capsules (e.g., hard gelatin capsules of 200 mg). Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. Additionally, tablets or capsules containing stenoparib or a pharmaceutically acceptable salt thereof or a hydrate thereof may include croscarmellose sodium and/or magnesium stearate.

Stenoparib or a pharmaceutically acceptable salt thereof or a hydrate thereof can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

### Dovitinib and Pharmaceutically Acceptable Salts

A subject as described herein may be treated for a cancer by administration of dovitinib or a pharmaceutically acceptable salt of dovitinib. Dovitinib or a pharmaceutically acceptable salt thereof or a hydrate thereof may be administered in a dosage form. Pharmaceutically acceptable salts of dovitinib described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of dovitinib described herein or separately by reacting a free base group with a suitable organic acid.

Dovitinib may have ionizable groups so as to be capable of preparation as a pharmaceutically acceptable salt. These salts may be acid addition salts involving inorganic or organic acids or the salts may, in the case of acidic forms of dovitinib be prepared from inorganic or organic bases. Frequently, dovitinib may be prepared or used as a pharmaceutically acceptable salt prepared as an addition product of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. In a particular example, the salt form of stenoparib is dovitinib lactate (e.g., dovitinib lactate, monohydrate).

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

Dovitinib or a pharmaceutically acceptable salt thereof or a hydrate thereof can be administered in a pharmaceutical composition that includes one or more pharmaceutically acceptable carriers, excipients, or diluents. Examples of suitable carriers, excipients, or diluents of dovitinib or a pharmaceutically acceptable salt thereof or a hydrate thereof are described in the Handbook of Pharmaceutical Excipients, 6th Edition, Rowe et al., Eds., Pharmaceutical Press (2009), hereby incorporated by reference in its entirety.

A pharmaceutical composition can be formulated to be compatible with its intended route of administration. Oral compositions can include an inert diluent or an edible carrier. The composition can be enclosed in a gelatin capsule or compressed into a tablet. For the purpose of oral therapeutic administration, dovitinib or a pharmaceutically acceptable salt thereof or a hydrate thereof can be incorporated with excipients and used in the form of tablets, troches, or gelatin capsules. In particular, dovitinib or a pharmaceutically acceptable salt thereof or a hydrate thereof can be formulated as hard gelatin capsules (e.g., hard gelatin capsules of 10 mg, 25 mg, 50 mg, 100 mg, or 200 mg) or film coated tablets (e.g., film coated tablets of 10 mg, 50 mg, 100 mg, 200 mg, or 250 mg). In particular, dovitinib or a pharmaceutically acceptable salt thereof or a hydrate thereof can be formulated as film coated tablets (e.g., film coated tablets of 100 mg). Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. Additionally, tablets or capsules containing dovitinib or a pharmaceutically acceptable salt thereof or a hydrate thereof may include microcrystalline cellulose, crospovidone, silica anhydrous, magnesium stearate, and/or a film-coating.

Dovitinib or a pharmaceutically acceptable salt thereof or a hydrate thereof can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

### DRP® Companion Diagnostic for Stenoparib

The methods described herein include administration of stenoparib monotherapy or stenoparib and dovitinib combination therapy to a subject with a cancer who may have sensitivity or resistance to treatment including stenoparib or a pharmaceutically acceptable salt thereof or a hydrate thereof. The methods may include determining, prior to therapy, whether the subject is likely to respond to the therapy, e.g., using a DRP® companion diagnostic for stenoparib.

A subject with a cancer (e.g., a subject with a newly diagnosed cancer or a recurrence thereof) can be assessed for sensitivity or resistance to stenoparib or a pharmaceutically acceptable salt thereof or a hydrate thereof by detecting expression of a biomarker (e.g., one or more of the biomarkers of Table 1 and/or Table 2) in a biological sample obtained from the subject. The biological sample can include, for example, cells, tissue (e.g., a tissue sample obtained by biopsy), blood, serum, plasma, urine, sputum, cerebrospinal fluid, lymph tissue or fluid, or pancreatic fluid. For example, the biological sample can be fresh frozen or formalin-fixed paraffin embedded (FFPE) tissue obtained from the subject, such as a tumor sample (e.g., a biopsy) from the tissue of interest (e.g., lymph nodes, thymus, spleen, bone marrow, breast, colorectal, pancreatic, cervical, prostate, bladder, lung, gastrointestinal, head, neck, or ovarian tissue).

Table 1 below lists biomarkers for sensitivity to treatment with stenoparib or a pharmaceutically acceptable salt thereof or a hydrate thereof, which are included in the stenoparib-DRP®companion diagnostic. Table 2 below lists biomarkers for resistance to treatment with stenoparib or a pharmaceutically acceptable salt thereof or a hydrate thereof, which are included in the stenoparib-DRP® companion diagnostic.

TABLE 1
Biomarkers of sensitivity to stenoparib. Dashes mean that
the Affymetrix probeset has not been mapped to a specific
gene. Affymetrix IDs refer to the array type HG-U133_Plus_2.

| | | | | SEQ |
|---|---|---|---|---|
| | Affymetrix | Cor- | | ID |
| Gene | ID | relation | Affymetrix Probe Sequence | NO: |
| SRSF7 | 214141_x_at | 0.516 | GATCAAGATCTATCTCTCTTCGTAG | 1 |
| UCHL 1 | 201387_s_at | 0.494 | GAGGGACTTTGCTGATTTCCCCTCT | 2 |
| MLLT11 | 211071_s_at | 0.491 | TCAGTGGGCACAGTTCTTCAGCTAC | 3 |
| ADD2 | 205268_s_at | 0.487 | CCTGGAGCAATGCACTGTACCTGCC | 4 |
| ADD2 | 213484_at | 0.451 | GTCTGTGTCTGACAATCGTCACGCA | 5 |
| PRMT1 | 206445_s_at | 0.441 | TCCCCGTACACGCACTGGAAGCAGA | 6 |
| SRSF3 | 202899_s_at | 0.439 | TTGAAGATCCCCGAGATGCAGCTGA | 7 |
| PRMT5 | 217786_at | 0.412 | GCGTTTCTGGCGATGCAGCAATTCC | 8 |
| COCH | 205229_s_at | 0.402 | AACCATGCCTACTAAATGTACAGAT | 9 |
| RUVBL1 | 201614_s_at | 0.391 | AGATCATTAAAATCCGTGCCCAGAC | 10 |
| MARCKSL1 | 200644_at | 0.39 | TTACTCAAGTTCAAACCTCCAGCCT | 11 |
| CHERP | 202230_s_at | 0.39 | TGTTCTAGATTTCCTGTAGCTGTGA | 12 |
| MTSS1 | 203037_s_at | 0.39 | GTGCTTTAGTTGCTAGTTTGTACTG | 13 |
| LSM4 | 202736_s_at | 0.385 | CCTGCGAGAAGTCATCTGCACGTCC | 14 |
| RAPGEF5 | 204681_s_at | 0.379 | GCCATTAACAGTGTTTTCTTTCCCA | 15 |
| PRPF4 | 209161_at | 0.367 | CGCAGCCACGCCGATGGTTATACAG | 16 |
| LSM4 | 202737_s_at | 0.366 | TCTTGCTAAAACCGGCAATTCTCCG | 17 |
| DESI2 | 212371_at | 0.365 | TGACTCCATTTCTGTAAGCTACTCT | 18 |
| RNPS1 | 200060_s_at | 0.363 | GGTGGTCTTTCAGGTTATCTTGGCA | 19 |
| SNX10 | 218404_at | 0.362 | TGTGTCTTAGATCCTCATTATTTTA | 20 |
| CUL3 | 201371_s_at | 0.361 | CTGTACTCTTCGCATGGACTGGGAA | 21 |
| CHD4 | 201184_s_at | 0.36 | CGGCTCTAGCCACTGAGCGGCTAAA | 22 |
| MSH2 | 209421_at | 0.359 | AAGATTGGTGCTTTTTGCATGTTTG | 23 |
| HNRNPM | 200072_s_at | 0.358 | GCCGAGAGAGCCTGCCGGATGATGA | 24 |
| SRSF1 | 211784_s_at | 0.358 | GGAGCAATCTCCTTGGAAGGATTCA | 25 |
| NELL2 | 203413_at | 0.357 | GAAAAGTCATCCCTATTTCCTTGTT | 26 |
| PAICS | 201014_s_at | 0.356 | AGTGTTGATGGGCTCTACTTCTGAT | 27 |
| HOXA10 | 213150_at | 0.356 | ATTTACCTCTAAATTTCCATATCCT | 28 |
| BUB1B | 203755_at | 0.354 | GGCCTTGTCTAACTTTTGTGAAGAA | 29 |
| E2F5 | 221586_s_at | 0.352 | CCACAGAAATCCAGCATGGCAACTC | 30 |
| MAGED4 | 221261_x_at | 0.351 | AAGATGAAGCTCTCTTTGCTCTTCC | 31 |
| MAGED4B | | | | |
| SNORA11D | | | | |
| SNORA11E | | | | |
| PRPF8 | 200000_s_at | 0.349 | GAGGGGGAGGTTTACTCTGCGGATC | 32 |
| SORD | 201563_at | 0.348 | ATCGCATCATGCCAACTTGTGACTT | 33 |
| HNRNPU | 200593_s_at | 0.346 | TTTTCCTGGCCGTGGTAGTTACTCA | 34 |
| PEX5 | 203244_at | 0.344 | TCATCCTCTTAGAAGCACCTGTGAA | 35 |
| HYPK | 218680_x_at | 0.344 | GGTAGAGGCGCTTATTGCCCTAACC | 36 |
| MIR1282 | | | | |
| SERF2 | | | | |
| SERF2- | | | | |
| C15ORF63 | | | | |
| STRAP | 200870_at | 0.343 | TCTTTCCTTCAGCTCCTGATGTTAA | 37 |
| NDUFAB1 | 202077_at | 0.341 | CATCCAGGACCGTGTTCTTTACGTA | 38 |
| FARSA | 216602_s_at | 0.341 | TATTCCGGAATGAGACCCTGGACGC | 39 |
| STOML2 | 215416_s_at | 0.339 | GCTGCAGCTCTGACACAACATAATG | 40 |
| ERH | 200043_at | 0.337 | GAGGATCTTGTTCAATCGGAAACCC | 41 |
| HSBP1 | 200941_at | 0.337 | TGATGTTCTCAAGCCTCGGAAGTGG | 42 |
| DDX39A | 201584_s_at | 0.337 | TTAACTACGACATGCCTGAGGACTC | 43 |
| ODC1 | 200790_at | 0.335 | CCAGAGGCCGACGATCTACTATGTG | 44 |
| TAF5 | 210053_at | 0.334 | GCTACATACAACCTAGTACACTTGA | 45 |
| TBC1D31 | 214061_at | 0.334 | AATGCGGCTGTAGAACATGCTGAAA | 46 |
| TRA2B | 200892_s_at | 0.333 | CCTTCTCCTTACTATAGTCGTGGAG | 47 |
| NUDC | 201173_x_at | 0.333 | CACAGGTCCCGGGGCATCAGGAGAA | 48 |
| DDX23 | 201440_at | 0.333 | CCAAGGGGTGCTGTATGCTCTAGGC | 49 |
| PRPF31 | 202407_s_at | 0.333 | CAGTTTCCACGAGAGCACAGAAGGG | 50 |
| UBE2S | 202779_s_at | 0.333 | GGCGCCAATGGCGAGATCTGCGTCA | 51 |
| TCF4 | 222146_s_at | 0.332 | GAGAAGGAGCGGAGGATGGCCAACA | 52 |
| MLF2 | 200948_at | 0.33 | GGGTCAATGCATTTTGGGGTGAGCT | 53 |
| CCDC181 | 206721_at | 0.329 | CAAACCTTTTCGTTTTACTGATCAT | 54 |
| TCF4 | 213891_s_at | 0.328 | TTGGTCAAGGCCATTACCTGTTTCC | 55 |
| DESI2 | 222158_s_at | 0.328 | TATACCCTTTCTACAGAGTTGCCTC | 56 |
| RPF1 | 218462_at | 0.327 | GCTGAACAGGCCTATCTTGAACTTT | 57 |
| PASK | 216945_x_at | 0.326 | TAATGGCCAAGGCTGTTTGCATCCC | 58 |
| NUP88 | 202900_s_at | 0.325 | AGCGAAAGTGCATTCAGTCCATCCT | 59 |
| RNASEH2A | 203022_at | 0.325 | ACTGATTATGGCTCAGGCTACCCCA | 60 |
| FBL | 211623_s_at | 0.325 | GTGATCGAGGATGCTCGACACCCAC | 61 |
| LOC101928747 | 213762_x_at | 0.325 | GGCAGGCAATTCTAAGATCTTCCAC | 62 |
| RBMX | | | | |
| SNORD61 | | | | |
| NXF1 | 208922_s_at | 0.324 | GTGATCGTAGTCATGCCTCAGAAGC | 63 |
| PLEKHO1 | 218223_s_at | 0.323 | GATGGGAAGCGAAAGGCCAAGGACC | 64 |
| GAR1 | 219110_at | 0.323 | ACAGACATCACCAGTTGACTTCTGC | 65 |
| RPA1 | 201529_s_at | 0.321 | GTAACTAGGTTTTTGCTTCTCCAGT | 66 |
| ZNF24 | 212534_at | 0.321 | CACAGCTTCCTAGCCACAGTGATAA | 67 |
| BOP1 MIR7112 | 216397_s_at | 0.321 | CGGCAGCGCAAGATGAGGGTGAATG | 68 |
| RAB3B | 205924_at | 0.32 | AGATACTCAGCACTAGACTAACATA | 69 |
| SLC35G2 | 219569_s_at | 0.319 | CATTGCTATATGTGTCTGTTCTACT | 70 |
| TSPAN3 | 200973_s_at | 0.318 | GAGACAGCTGCATTGTTGTGGAATT | 71 |
| DKC1 | 201479_at | 0.318 | GCCTGATGGGCCTGGTAGTTTTCCA | 72 |
| MIR664B | | | | |
| SNORA56 | | | | |
| PSMC3IP | 213951_s_at | 0.318 | GTAACCGTAGCAAAACTGCATTGGT | 73 |
| DNAJC7 | 202416_at | 0.316 | AGATCGGCATAGTGGAGCCAGTGCT | 74 |
| RRP1B | 212846_at | 0.316 | TTTGTAAAATGATGCTTCCCCCTTC | 75 |
| NME1 | 201577_at | 0.315 | AAGGAGATCGGCTTGTGGTTTCACC | 76 |
| SNRPA | 201770_at | 0.315 | ACAGCATTGTACCCAGAGTCTGTCC | 77 |
| DBN1 | 217025_s_at | 0.315 | TGCCCAATCGGAAGAGCTCTGTGCC | 78 |
| KIAA0020 | 203712_at | 0.314 | AGGTGCCATTATTCTTTCTAGCCTC | 79 |
| SUPV3L1 | 212894_at | 0.313 | GAAGCTGTCCACGATGTCTTGGATC | 80 |
| ZNF573 | 217627_at | 0.31 | GGAGCATAAAAGTGTAAGACCGTAA | 81 |
| FAM134B | 218510_x_at | 0.31 | GATCTTTCAGATTTTCCATCTCTAG | 82 |
| TOX3 | 214774_x_at | 0.309 | ATGACCACTGTTAGCCCATTATATT | 83 |
| HSPD1 | 200806_s_at | 0.306 | TAGTATCCAGTCCATTGTACCTGCT | 84 |
| ACLY | 210337_s_at | 0.306 | AGATGGTCTCATCGGAGTCGCATTT | 85 |
| TOX3 | 216623_x_at | 0.306 | ATATCTATGACCACTGTTAGCCCAT | 86 |
| MSANTD3- | 205122_at | 0.305 | TAAAGCCTATTCTACCAGTTAAACT | 87 |
| TMEFF1 | | | | |
| TMEFF1 | | | | |
| AKIRIN1 | 217893_s_at | 0.305 | GGAGCATCAGGGTTGGCTTGGGAGC | 88 |
| UBE2M | 203109_at | 0.304 | CATTGACCTCGAGGGCAACGTCTGC | 89 |
| MTF2 | 203345_s_at | 0.304 | TTATACGTTTATATGCTCTGTCTGC | 90 |
| EWSR1 | 209214_s_at | 0.304 | GTTGGCCACAACATTATGATTATTC | 91 |
| FARSA | 202159_at | 0.303 | TCCTGTGTGGTGTGTCTACTGTGAG | 92 |
| SKP2 | 203625_x_at | 0.303 | AGAGCTGGGGTTAGGATCCGGTTGG | 93 |
| TMEM97 | 212279_at | 0.303 | TCCTTTCTGTTTTGCGAGCTTGTGT | 94 |
| HNRNPD | 221481_x_at | 0.303 | AAAAGCCCAGTGTGACAGTGTCATG | 95 |
| ILKAP | 221548_s_at | 0.303 | CAGTACAAGCGCTGCGGTGTCACCT | 96 |
| NASP | 201970_s_at | 0.302 | GAAAGCCAACAGACGGTGCTTCCTC | 97 |
| SNRPD1 | 202690_s_at | 0.301 | GTTTACCTCTGGATACACTACTTGT | 98 |
| TIMM44 | 203093_s_at | 0.301 | CGCTCTGCCGAGACCAGGACGAGCT | 99 |
| PKN1 | 202161_at | 0.3 | CACCGACGTCAGCAACTTCGACGAG | 100 |
| STAU2 | 204226_at | 0.299 | AAGCCTACTAGAGCCATTGTATGTG | 101 |
| DNAAF2 | 219166_at | 0.299 | CTTCATTACTGAACAAAACTACGGT | 102 |
| SNRPD2 | 200826_at | 0.297 | TGCTCATCAACTGCCGCAACAATAA | 103 |
| FUS | 200959_at | 0.297 | ATCCCACCTGTGAGAATATGAACTT | 104 |
| PASK | 213534_s_at | 0.297 | AGCTAAACACCAATTTCTTCCTGCT | 105 |
| ATP6V1G2- | 200041_s_at | 0.296 | TAGTGAGCTGCCTGATGAGATAGAC | 106 |
| DDX39B | | | | |
| DDX39B | | | | |
| SNORD84 | | | | |
| PDSS1 | 220865_s_at | 0.296 | GCAGCTATCTTACCAGACTGTGCCT | 107 |
| NUDC | 210574_s_at | 0.295 | GAAGTTCATGGATCAACATCCGGAG | 108 |
| TOX3 | 215108_x_at | 0.295 | GGTGACATAAACCATTCATTGCTAC | 109 |
| TPGS2 | 212055_at | 0.294 | AAGTGCCCAAATAAGTCTGAGTGCT | 110 |
| SLIRP | 221434_s_at | 0.294 | TTTGACAAGGAGACTGGCTTTCACA | 111 |
| NCL | 200610_s_at | 0.293 | AAGTTTGAATAGCTTCTGTCCCTCT | 112 |
| ANP32A | 201051_at | 0.293 | TTTGGAGTTCTCTTACGTTTCCTGG | 113 |
| SAFB | 201748_s_at | 0.293 | TGGCGAGAGAAGCATGTCCGGTCAC | 114 |
| STIP1 | 212009_s_at | 0.293 | AATAAGTGCCGGGAGCTTTGTGAGA | 115 |
| CEP68 | 212677_s_at | 0.293 | GGCCAGAAACCTTGGGTCTTTTCAT | 116 |
| STIP1 | 213330_s_at | 0.291 | GGATGTGGGTCTGATTGCAATTCGG | 117 |
| C8orf33 | 218187_s_at | 0.291 | TGGACCATGGTCTAGCAGTAGCATC | 118 |
| MRPL11 | 219162_s_at | 0.29 | GGGCATTCGCGTGGTGAAGGACCTC | 119 |
| POLR21 | 212955_s_at | 0.288 | GCTGTACCCCAAGGAAGACAAGGAG | 120 |
| FAM134B | 218532_s_at | 0.288 | AAATCTCTGAACTGTGTGGGTTTTG | 121 |
| MCAM | 211042_x_at | 0.286 | TGCCCTCAAACATACAGAACTTCCA | 122 |
| MIR6756 | | | | |
| ECSIT | 218225_at | 0.285 | TCATCTCTAGCAGTATGGCATCCCC | 123 |
| MDK | 209035_at | 0.284 | GCAAGGATTGCGGCGTGGGTTTCCG | 124 |
| PUF60 | 209899_s_at | 0.283 | TCAATGGCCGCTGGTTTGCTGGCCG | 125 |
| PFN2 | 204992_s_at | 0.282 | CACACCAGCTCTACTCTTTAGTAAA | 126 |
| SYNCRIP | 209025_s_at | 0.282 | AGAAACTCACCCTAAATCTGAACGG | 127 |
| TSPAN3 | 200972_at | 0.281 | AAGTGGTAACAGGACTGATGCCGAA | 128 |
| SLC16A1 | 202236_s_at | 0.281 | TGTTTATAGATCACTGCCCTTTTTG | 129 |
| POLR2H | 209302_at | 0.281 | AAGTCACTCAGGTCATGGGCATTGT | 130 |
| MAP3K7 | 211537_x_at | 0.281 | TGATTACTACCTCAGGACCAACCTC | 131 |
| CSRP2 | 207030_s_at | 0.28 | GTACTTGGATAGGCTGGCTAACTCG | 132 |
| BCL11A | 210347_s_at | 0.28 | GAAGCTCGAGAGCCCTTAAGTTCTG | 133 |
| PNKP | 218961_s_at | 0.279 | CCATCGACAACACAAACCCAGACGC | 134 |
| DNAJC6 | 204720_s_at | 0.278 | AGCTTGTTGTTGATCAGATTCACTG | 135 |
| FDFT1 | 208647_at | 0.278 | GCAGAGCATTCAGTGCCACGGTTTA | 136 |
| FADS1 | 208963_x_at | 0.278 | CTCTTCAGCAGCATCTACTCTAGGC | 137 |
| MIR1908 | | | | |
| RPARP-AS1 | 214857_at | 0.277 | CTGCTGTAAGGACTGGTTCCAGAAT | 138 |
| DHRS7 | 210788_s_at | 0.276 | GAACTTGCCACATACCCAGGTATAA | 139 |
| CCNB1IP1 | 217988_at | 0.276 | CATGAAGGCACCCTTGAACCATCCA | 140 |
| CCT3 | 200910_at | 0.275 | GGAGCCATTGGCTGTGAAGCTGCAG | 141 |
| LOC101927137 | | | | |
| DDX18 | 208895_s_at | 0.274 | AATCTGACCCTATCGGAAACTCAAA | 142 |
| AARSD1 | 222064_s_at | 0.273 | ATAGCCTCAGGAACAGTCCAGACTG | 143 |
| PTGES3L | | | | |
| PTGES3L- | | | | |
| AARSD1 | | | | |
| HNRNPDL | 209067_s_at | 0.272 | GGTGGTGATCAAAACTATAGTGGCT | 144 |
| ATXN7L3B | 212114_at | 0.272 | TGATCACCCTGCAATCCTATTATGT | 145 |
| MRPS14 | 203801_at | 0.271 | ACTTAGAATCCATCTCACTACCAAT | 146 |
| SOX4 | 201416_at | 0.269 | TAGAGACTATGTCGCTTTCCTGAGC | 147 |
| ELOVL2 | 213712_at | 0.268 | TACTGTGATGGGCAGAACAGCCTTT | 148 |
| KCNJ8 | 205303_at | 0.267 | TCTCATTTAGGACTGGGTTCCCCAC | 149 |
| TRIAP1 | 218403_at | 0.267 | ACAGCTTTTAACACAGTTCCCTGCC | 150 |
| EIF2B1 | 201632_at | 0.266 | GATGAAAGATGAGCCCAAGTCCACC | 151 |
| FBXL14 | 213145_at | 0.266 | GATCCTCTTAAAACGTTTATATTCT | 152 |
| MAPRE2 | 213489_at | 0.266 | CCCTGGTCACATGTCATCGGGCTGG | 153 |
| ORC4 | 203352_at | 0.264 | ATCCACATCAGAATCCCTTGTAGGA | 154 |
| MDN1 | 212693_at | 0.264 | ACTGCTTTCAAGCTGGACTGAGCCA | 155 |
| KNOP1 | 213237_at | 0.264 | TAAACATTCCGTCCCTGTTTGAGAC | 156 |
| KBTBD11 | 204301_at | 0.263 | GTGAGATCAAAGCTCCTCCAAAGCC | 157 |
| FADS2 | 202218_s_at | 0.262 | CCTCGGTGGCCCTGACTGTCAGGGA | 158 |
| RANBP1 | 202483_s_at | 0.262 | TCACGCCGATGATGGAGCTGAAGCC | 159 |
| PLEKHB1 | 209504_s_at | 0.261 | GAGTTCTATTTGAGACTTCCAGCCC | 160 |
| HSPE1 | 205133_s_at | 0.26 | CCAAAGGAGGCATTATGCTTCCAGA | 161 |
| TMEM97 | 212282_at | 0.26 | GAAGTGTGCTAGTATGCTCCCTAGT | 162 |
| ITFG2 | 220589_s_at | 0.26 | GTTGGGGTTGGAGACGTGTGTAATA | 163 |
| LOC100507424 | | | | |
| SFPQ | 201586_s_at | 0.259 | GGAGGCTAAGTATTGCTTTCTACAA | 164 |
| RFC3 | 204127_at | 0.259 | TCATTAGCTCTGCCCTTTTTAATTA | 165 |
| SDR39U1 | 213398_s_at | 0.259 | CACTTCTCCTTTACGCTGTGTAGAG | 166 |
| PBK | 219148_at | 0.259 | GTACTTTGATACTGCTCATGCTGAC | 167 |
| PHB | 200658_s_at | 0.258 | CAGGGGATGGCCTGATCGAGCTGCG | 168 |
| KHDRBS1 | 201488_x_at | 0.258 | CCTTATTCCATTCTTAACTCTGCAT | 169 |
| PDAP1 | 202290_at | 0.256 | GCAGTCACTCTCCCTGAATAAGTAA | 170 |
| SSRP1 | 200957_s_at | 0.255 | GAAGAAGAACTAGCCAGTACTCCCC | 171 |
| B3GALT2 | 210121_at | 0.251 | GGAGTGCCAAGTTTGCAAACCTGCA | 172 |

TABLE 2
Biomarkers of resistance to stenoparib. Dashes mean that
the Affymetrix probeset has not been mapped to a specific
gene. Affymetrix IDs refer to the array type HG-U133_Plus_2.
All correlations are negative.

| | Affymetrix | Cor- | | SEQ |
|---|---|---|---|---|
| Gene ID | ID | relation | Probe sequence | ID no |
| HLA-E | 200904_at | 0.488 | AATGGGTTATCACAGGAATGGGACT | 173 |
| HLA-E | 200905_x_at | 0.477 | TGGGCAGAGTGCGGCAGCTCATGCC | 174 |
| GADD45B | 209304_x_at | 0.47 | TGGAGTGAGACTGACTGCAAGCCCC | 175 |
| CLIC1 | 208659_at | 0.454 | TTTTTGGATGGCAACGAGCTCACCC | 176 |
| LASP1 | 200618_at | 0.451 | TTATTGCTTAATTTCTGCCTTTCCC | 177 |
| HLA-E | 217456_x_at | 0.445 | GCTGCCTTGTGTGCGACTGAGATGC | 178 |
| APOBEC3B | 206632_s_at | 0.441 | CTACGATGAGTTTGAGTACTGCTGG | 179 |
| LGALS1 | 201105_at | 0.435 | CTCCTGGACTCAATCATGGCTTGTG | 180 |
| TAPBP | 208829_at | 0.435 | TGGAGGGCGCTCATCAAGTAGCTGC | 181 |
| AHNAK | 211986_at | 0.432 | TGGTCCCAGCCAGTTTGGTGCTGAC | 182 |
| BHLHE40 | 201170_s_at | 0.427 | GATCCTTTCTGTAGGCTAATTCCTC | 183 |
| S100A11 | 200660_at | 0.423 | TTCCCAGAAGCGGACCTGAGGACCC | 184 |
| LITAF | 200704_at | 0.413 | GACCTTCAAGGGTCCTCGTTTTGAT | 185 |
| ZBTB38 | 219221_at | 0.409 | GACGGTGCTTTTCGGTGCAAGGAAA | 186 |
| STAT1 | 209969_s_at | 0.408 | TGAACTTGTTGAGATCCCCGTGTTA | 187 |
| TCIRG1 | 204158_s_at | 0.404 | TTCTACTCAGGCACGGGCTACAAGC | 188 |
| S100A11P1 | 208540_x_at | 0.397 | CCGGTGTCCTTGACCACATGAAGAA | 189 |
| CTSA | 200661_at | 0.39 | TGATGCACTGATTCCATCCCAGGAA | 190 |
| VEGFA | 210512_s_at | 0.39 | TGACAGTCACTAGCTTATCTTGAAC | 191 |
| STOM | 201061_s_at | 0.388 | TTGTGTGAAGTGGTTCACCCTTGAG | 192 |
| P4HB | 200654_at | 0.387 | GACTTCCGGATCCTGTCAGGGTGTC | 193 |
| LITAF | 200706_s_at | 0.387 | GACAAAGATCTTGCCTTACAGACTT | 194 |
| FXYD5 | 218084_x_at | 0.384 | TGGTCGCAGCTGTGCTGTTCATCAC | 195 |
| HLA-C | 208812_x_at | 0.382 | GTGACTTCAAGAGCCTCTGGCATCT | 196 |
| YPEL5 | 217783_s_at | 0.38 | GAGAACAACAGCATTCATTTCCATT | 197 |
| HLA-C | 214459_x_at | 0.379 | CCCTCACCCTGAGATGGGAGCCGTC | 198 |
| HLA-C | 211799_x_at | 0.374 | CACCATCCCCAACTTGGGCATCGTT | 199 |
| STOM | 201060_x_at | 0.373 | AGTAAATGATTCCTCCTTGTTCTGT | 200 |
| PLIN3 | 202122_s_at | 0.373 | CAAAACGGGCATCACCCAGTTGACC | 201 |
| RRBP1 | 201206_s_at | 0.37 | AAGGAGGGCACCTCTGTCTGAGTTT | 202 |
| RF1 | 202531_at | 0.37 | TTATTTATACAGTGCCTTGCTCGGG | 203 |
| LMNA | 212086_x_at | 0.37 | TTCGGGGACAATCTGGTCACCCGCT | 204 |
| NPC2 | 200701_at | 0.369 | CCTCATTGAGTTCGGTGCATCTGGC | 205 |
| P4HB | 200656_s_at | 0.368 | TGCTTGCTACCGTGTTCGGAGTCTC | 206 |
| KLF6 | 208961_s_at | 0.367 | CCCTGCACATGAAGAGGCACCTCTG | 207 |
| HLA-B | 209140_x_at | 0.365 | GGCTGTCCTAGCAGTTGTGGTCATC | 208 |
| RHOC | 200885_at | 0.362 | GTCACACACCAGCACTTTATACACT | 209 |
| CD59 | 200985_s_at | 0.362 | GAACACTCTACTACATGTGACTGGC | 210 |
| SRGN | 201858_s_at | 0.362 | GAGGACTACTCTGGATCAGGCTTCG | 211 |
| SRGN | 201859_at | 0.359 | GAGCTAGTGGATGTGTTTGTCTACA | 212 |
| STAT1 | 200887_s_at | 0.358 | TTCCTTCCTTATCACTGACACAAAA | 213 |
| TNFSF10 | 202687_s_at | 0.358 | GGACTCTATTCCATCTATCAAGGGG | 214 |
| HLA-B | 211911_x_at | 0.358 | CCCCATCGTGGGCATTGTTGCTGGC | 215 |
| PIEZO1 | 202771_at | 0.357 | GCGGATTCTTCAGCGAGATCTCGCA | 216 |
| LGALS3 | 208949_s_at | 0.355 | CTCACCAGTGCTTCATATACCATGA | 217 |
| LDLRAP1 | 221790_s_at | 0.355 | GCAAGTTTCATCAGCCCTAGGGAAA | 218 |
| CD97 | 202910_s_at | 0.353 | CTCAGCTTCCCTCTTAAGCTAAGAC | 219 |
| HLA-B | 208729_x_at | 0.353 | CCCCATCGTGGGCATTGTTGCTGGC | 220 |
| CFLAR | 209939_x_at | 0.353 | GAATGTTCTCCAAGCAGCAATCCAA | 221 |
| FNDC3B | 218618_s_at | 0.353 | ATGTGCGTGTCGTCGCTGTTTAGAC | 222 |
| LOC101928615 | | | | |
| CKLF | 219161_s_at | 0.353 | CACAAGCCCCTGAACCATATATTGT | 223 |
| CKLF-CMTM1 | | | | |
| IFI35 | 209417_s_at | 0.351 | GATGTGGACGTTCGGGAGCTACTGC | 224 |
| TIPARP | 212665_at | 0.35 | TCCTGTTGTTTGCTGCCATTGGCAT | 225 |
| TAP1 | 202307_s_at | 0.348 | GTGGCCAGCACTCTGAAACTGAGAA | 226 |
| MICALL2 | 219332_at | 0.348 | AGTTCCGCTTGTCCAAGATCTGGTC | 227 |
| RRBP1 | 201204_s_at | 0.347 | AGAGCCCCAGTTTGTAAATGAACCT | 228 |
| ZFP36 | 201531_at | 0.347 | GTCCCCAAGTGTGCAAGCTCAGTAT | 229 |
| HLA-G | 211528_x_at | 0.347 | GACGTGGAGCTCGTGGAGACCAGGC | 230 |
| TNIP1 | 207196_s_at | 0.346 | TTTTTGCTTCAAGCTCTGTAGCAGG | 231 |
| CD59 | 200983_x_at | 0.345 | TCCATTTCTGGCAGCAGCCTGGAGC | 232 |
| VEGFA | 212171_x_at | 0.345 | TCTACCCCAGGTCAGACGGACAGAA | 233 |
| LDLRAP1 | 57082_at | 0.345 | ACGGGCTGTCCGTGTGCCTCCTGTG | 234 |
| FLNB | 208613_s_at | 0.343 | GGTGGAGTCAGTGACCAGGTCGTCT | 235 |
| PSG6 | 209738_x_at | 0.343 | CTAACCCACCGGCAGAGTATTTTTG | 236 |
| CBX7 | 212914_at | 0.342 | GCTTTCGAAGTGGCCAGCTGCGGCC | 237 |
| RARRES3 | 204070_at | 0.339 | GTGTTAGAAGCAGCTGTGGGGGTCC | 238 |
| CFLAR | 210563_x_at | 0.339 | GAGTGAGGCGATTTGACCTGCTCAA | 239 |
| SUN2 | 212144_at | 0.337 | TAAGGGCTTGATTCCTAGCCCCGTT | 240 |
| EHD2 | 45297_at | 0.337 | AGGGGCCTCTGAACGGAACAGTGTC | 241 |
| MAP3K5 | 203836_s_at | 0.336 | TGAGCACGCTCAGTTCTACTGTGTC | 242 |
| BTN3A2 | 209846_s_at | 0.336 | GTCTTCGTCCGATACCAATAAGTCA | 243 |
| NOL12 TRIOBP | 210276_s_at | 0.336 | AGCTGAGCCACATCAAGACACGGTC | 244 |
| CKLF | 221058_s_at | 0.336 | CTTATTTACCGGAAGCTTCTGTTCA | 245 |
| ARPC1B | 201954_at | 0.335 | TGCCTTCATCCTAACTGCTGGGGAA | 246 |
| TNFSF10 | 202688_at | 0.335 | GAGGCACCACTAAAAGATCGCAGTT | 247 |
| HLA-G | 211529_x_at | 0.335 | TACCCTGCGGAGATCATACTGACCT | 248 |
| RP11-395B7.7 | 214808_at | 0.335 | GGATGGGAAGTGTTGCATTGAGCAT | 249 |
| EHD2 | 221870_at | 0.335 | CTTGGAGATATTTCCGTCCTCCACC | 250 |
| LEPROT | 202378_s_at | 0.334 | AGGCCTCTCATGACCCAGGAAGGCC | 251 |
| BTN3A2 | 204820_s_at | 0.333 | TCATCACTATTATTGCTCACCACTG | 252 |
| BTN3A3 | | | | |
| INPP4B | 205376_at | 0.332 | AAGCTGACACCTAAGTTTACCAACA | 253 |
| DUSP1 | 201041_s_at | 0.331 | TAGGTGTCTGCCTTCACAAATGTCA | 254 |
| EVI2A | 204774_at | 0.33 | ATATTCCCTATGTAGCAACAGTGGT | 255 |
| TFPI | 213258_at | 0.33 | GATCATGCTGAAAACCACTCAAACG | 256 |
| EHD1 | 209037_s_at | 0.329 | AAGGTCCATAAAGACTGAGCGGATG | 257 |
| VEGFA | 210513_s_at | 0.329 | CTGAGGAGTCCAACATCACCATGCA | 258 |
| EPAS1 | 200878_at | 0.328 | GGACGACACCTCTGGTTTTTCAATA | 259 |
| IQGAP1 | 210840_s_at | 0.328 | TTCACTCCATCCCTATGGCAGAGGA | 260 |
| IL6ST | 212195_at | 0.327 | GAGATGCATTTAAGGCCGATAGTGA | 261 |
| CLIC3 | 219529_at | 0.327 | TCGTTACAGGGAGTCCAACACCGCC | 262 |
| TFPI | 210664_s_at | 0.326 | GATGGTCCGAATGGTTTCCAGGTGG | 263 |
| NACC2 | 212993_at | 0.326 | GTTTTTGTGCTGTTACTCGGTAGAG | 264 |
| TGFBI | 201506_at | 0.325 | GAGATGTGAGCCTTGTGCATGTGGG | 265 |
| IER3 | 201631_s_at | 0.325 | TGAGATCCGTGAGATCCTTCCATCT | 266 |
| MICA | 205904_at | 0.325 | TATTATTTTCTATGTCCGTTGTTGT | 267 |
| BTN3A2 | 212613_at | 0.325 | TACTGGGCAAGTGCTTGTCAAGTTC | 268 |
| IQGAP1 | 200791_s_at | 0.323 | GAGGATGCCCCAACAAACTCATGGC | 269 |
| CNN2 | 201605_x_at | 0.321 | TCTGAGTTGTTGGGGCTAAGCCTGA | 270 |
| TNFAIP8 | 208296_x_at | 0.321 | GAAAGGTAACAATCTTCATTCTACA | 271 |
| VEGFA | 211527_x_at | 0.321 | ATCGATACAGAAACCACGCTGCCGC | 272 |
| MBNL1 | 201152_s_at | 0.32 | ACTCTTGAGGGTTGATTATGCTGCA | 273 |
| ISG15 | 205483_s_at | 0.318 | CCCTGAGCACCGTGTTCATGAATCT | 274 |
| TNFAIP8 | 210260_s_at | 0.318 | GATTGAGTCATCGACATTCAGGATT | 275 |
| COPG1 | 217749_at | 0.318 | CACCCGGGACTACTTGCTGGTGACT | 276 |
| CD99 | 201028_s_at | 0.317 | TGGAGAAAATGACGACCCACGACCA | 277 |
| PSMB9 | 204279_at | 0.317 | GACAGCCTTTTGCCATTGGTGGCTC | 278 |
| ZFP36L1 | 211962_s_at | 0.317 | GGCCTTTCACAACTAGGACTGAGAA | 279 |
| IL6ST | 212196_at | 0.317 | TATGTAATGTACACCTGCCCTAAGG | 280 |
| SHC1 | 214853_s_at | 0.316 | GGGAGAGTTGACCGTTTTCATCCTG | 281 |
| GSTK1 | 217751_at | 0.316 | CATAAGGCACTGGGACTCGGATTTC | 282 |
| CAV1 | 203065_s_at | 0.315 | GCACATCTGGGCAGTTGTACCATGC | 283 |
| HLA-F | 204806_x_at | 0.314 | CACTGACAGTGCCCAGGGCTCTGGG | 284 |
| KRT7 | 209016_s_at | 0.314 | GAGTGGGAGCCGTGAATATCTCTGT | 285 |
| TFPI | 209676_at | 0.314 | AAATTATCATACTACCGGCTACATC | 286 |
| SPTBN1 | 212071_s_at | 0.314 | TAATTTGCTTCATTTCCTTGCTATT | 287 |
| RHOG | 203175_at | 0.313 | AAGGTGAATGTACCCTAACCTGCTC | 288 |
| CDH11 | 207173_x_at | 0.313 | GAGCATCAATCTTGTTACTGCTGAT | 289 |
| ABCC3 | 208161_s_at | 0.313 | GATCCCCAAGTGGTGAATGACACGC | 290 |
| CAV1 | 212097_at | 0.313 | GTTACCTACTTTGACTTTTTGCATT | 291 |
| HLA-J | 217436_x_at | 0.313 | GAACGGGAAGGAGACGCTGCAGCGC | 292 |
| MYL12A | 201319_at | 0.312 | CAGACTGGAAACGGGACTTTCTATT | 293 |
| MRPS10 | 218106_s_at | 0.312 | AACCCCAGAGAAGTGCTTCATTTTC | 294 |
| RRAS | 212647_at | 0.309 | GACGAGGCTTTTGAGCAGCTGGTGC | 295 |
| TMEM2 | 218113_at | 0.309 | GCAAAATGCCTTTACGTTGTTCTAA | 296 |
| SIDT2 | 218765_at | 0.308 | GGGTCTGGATCTTTTCTCAGAGCGT | 297 |
| RAB11FIP1 | 219681_s_at | 0.308 | GGATTTCGTTTGCTGTGGCATTGGT | 298 |
| RTP4 | 219684_at | 0.308 | AACATCTGTGTCTTTATTTTGCTGC | 299 |
| LOC101928916 | 202238_s_at | 0.307 | TCCCCAGTGGTGACCTATGTGTGTG | 300 |
| NNMT | | | | |
| SPTBN1 | 200671_s_at | 0.306 | ATGACAGTTTCACAACCTGCATTGA | 301 |
| TMEM189 | 201003_x_at | 0.306 | GGGAACTTACTTCTCTATAGCCCAC | 302 |
| TMEM189- | | | | |
| UBE2V1 | | | | |
| UBE2V1 | | | | |
| UBE2V2 | | | | |
| RPN2 | 213399_x_at | 0.305 | GCTCCTAGCACGATTATATTTCACC | 303 |
| ITGA5 | 201389_at | 0.304 | CACTAACTATGCATGGTGCCCCAGG | 304 |
| CDC42EP1 | 204693_at | 0.304 | GGCTTGCTCTGGGACTTTTATGCTC | 305 |
| BTN3A3 | 204821_at | 0.304 | GAGAATAACCTCATAGTACCAACAT | 306 |
| OSER1 | 209020_at | 0.304 | GGCTCCAGAACGAAGATCCACACTT | 307 |
| CHST15 | 203066_at | 0.303 | GGATTTTTCTTATGCACTCCTATGC | 308 |
| MDFIC | 211675_s_at | 0.303 | AAACATGTACTGGCACAATTTGTGA | 309 |
| CAV2 | 203324_s_at | 0.302 | AAGGCAGACAGTTTTGCTTTAGTAC | 310 |
| CARD10 | 210026_s_at | 0.302 | ACGGGCCTTTTTCACTTAGAAGCTG | 311 |
| RAC2 | 213603_s_at | 0.302 | GAGTGTCTCAGAAGTGTGCTCCTCT | 312 |
| MLPH | 218211_s_at | 0.302 | GCTCTGTGCTTTCCACTATACACAG | 313 |
| F2R | 203989_x_at | 0.301 | GGTTGAAACATATCTCTTATCTTAC | 314 |
| ICAM3 | 204949_at | 0.301 | TGGTACTTATCAGTGCCAAGCGTCC | 315 |
| CRIM1 | 202552_s_at | 0.3 | GTAAGAGAATTATCTGCAACTTGAT | 316 |
| LOC101929500 | | | | |
| IFI16 | 208966_x_at | 0.3 | TTCATAGTCACATCAAGGTCATCAA | 317 |
| EVI2B | 211742_s_at | 0.3 | GGAACTATCTGAAATCTGCTCAGAG | 318 |
| PFKFB3 | 202464_s_at | 0.299 | GCAGTTGCCAGACCAATAAAATACC | 319 |
| MIR6513 | 217730_at | 0.299 | GGGAGATACCGAAGCCTACTGTGGT | 320 |
| TMBIM1 | | | | |
| APOL3 | 221087_s_at | 0.299 | TGGAAGTGTATCTCAGTCAGCCAGT | 321 |
| CD55 | 201925_s_at | 0.298 | GATCTGTAATGTTATTTCCACTTAT | 322 |
| TRAM2 | 202369_s_at | 0.298 | TTTTCTACTCTAGCCTGTTCATATG | 323 |
| S100A4 | 203186_s_at | 0.297 | GGCTTCCCAGATAAGCAGCCCAGGA | 324 |
| SERPINB1 | 212268_at | 0.297 | GTGGACTAAACCTGAGAATCTCGAT | 325 |
| PIP4K2A | 212829_at | 0.297 | GTGGTTTGACCCAAGACCACAACCA | 326 |
| RPN2 | 213491_x_at | 0.297 | GGTGAGCTCAGATAGTCTCTTTCTC | 327 |
| ALDOA | 214687_x_at | 0.297 | TCGTCTCTAACCACGCCTATTAAGC | 328 |
| IFIT3 | 204747_at | 0.296 | GCATAGGCAGTATTTTCCTGTCAGC | 329 |
| PLAC8 | 219014_at | 0.296 | AATGAGGACTCTCTACAGGACCCGA | 330 |
| SDF4 | 221972_s_at | 0.296 | CAAGTACAGCGAGTTCTTCACGGGC | 331 |
| CAV2 | 203323_at | 0.294 | GATGGGACGCATAATCATTACCTTA | 332 |
| HLA-C | 216526_x_at | 0.294 | CGATCATCTTTCCTGTTCCAGAGAG | 333 |
| MVP | 202180_s_at | 0.292 | GAGCTCCAGAGGGTCCAGAAGGTCC | 334 |
| RNH1 | 206050_s_at | 0.292 | TCAGCAACAACTGCCTGGGGGACGC | 335 |
| EIF1 | 212225_at | 0.292 | AGGGGTGATAAATGCGTTCATGCTC | 336 |
| SERPINB1 | 213572_s_at | 0.292 | TGCGTAGATTCTTGACCATGTAGTA | 337 |
| ASL | 204608_at | 0.291 | AAGCTGTGTTCATGGCCGAGACCAA | 338 |
| CD99 | 201029_s_at | 0.29 | TGTTCACGGCGGATTCTTTGTTTTA | 339 |
| USP4 | 202682_s_at | 0.289 | GAACATCTTTGACACTCTGCAGACT | 340 |
| TACC1 | 200911_s_at | 0.288 | TTCCCACGGGTGCCATGAAGTGTGT | 341 |
| CD55 | 201926_s_at | 0.288 | GGTCCCACCAACAGTTCAGAAACCT | 342 |
| PDXK | 218019_s_at | 0.288 | GAGGCATCAAAGCACCTGTCGCCTC | 343 |
| BST2 | 201641_at | 0.287 | AACATTCCCTTGATCTCATCAGTTC | 344 |
| LOC101928916 | 202237_at | 0.287 | CAACAACGAAGGACTTTTCTCCCTG | 345 |
| NNMT | | | | |
| DUSP5 | 209457_at | 0.287 | TGTGTAGACACCCTCTTGGGTCCAA | 346 |
| TNFSF13 | 210314_x_at | 0.287 | CCACAAGAAGCCTTATCCTACGTCC | 347 |
| COMT | 208818_s_at | 0.286 | TGAGTGCACACACTACCAATCGTTC | 348 |
| CYR61 | 210764_s_at | 0.286 | GATGCGGTTCCGCTGCGAAGATGGG | 349 |
| LY6E | 202145_at | 0.284 | GTCAGTAGGGATGTGTGCCTGGCTG | 350 |
| ACSL5 | 218322_s_at | 0.284 | CTCTCTAGTTAGATATCTGACTTGG | 351 |
| GBP2 | 202748_at | 0.283 | ACAGGGCCCAGTTAATGGCAGAGCA | 352 |
| TNFRSF1B | 203508_at | 0.283 | GAAGCGATGAATTTGGAGACTCTGT | 353 |
| PTRF | 208790_s_at | 0.283 | GGGTCCCAGAGGAACGCTGGAGCCA | 354 |
| CYR61 | 201289_at | 0.282 | GGAGCACATGTTACTGCTTCATTTT | 355 |
| BTN3A1 | 209770_at | 0.282 | ATCATTATTATTGCTCTCCACTGTA | 356 |
| PLEC | 201373_at | 0.281 | CATGGGAAGACGAGGCCCTCGGGCC | 357 |
| CTNND1 TMX2- | 208407_s_at | 0.281 | TACCAGGGACTATCTTTGCTGCAGA | 358 |
| CTNND1 | | | | |
| TNFRSF14 | 209354_at | 0.281 | GTGAGGCCACAGTCATTGAGGCCCT | 359 |
| ABCC10 | 213485_s_at | 0.281 | GAGCCCACTTGCATTTTCATAGTTT | 360 |
| SELPLG | 209879_at | 0.28 | CACTGCACTGCCATTGTCTTTTGGT | 361 |
| GPX4 | 201106_at | 0.279 | GATCAAAGAGTTCGCCGCGGGCTAC | 362 |
| EDEM1 | 203279_at | 0.279 | AAGAAAATCCTGAGCTTTCCTGTCC | 363 |
| MIR6787 | 202856_s_at | 0.278 | GATCGTCGCCCGATCAGTGTTTTGA | 364 |
| SLC16A3 | | | | |
| DMBT1 | 208250_s_at | 0.278 | GCTACCGAGGCTGTGTGTTGAGGTC | 365 |
| PSMB8 | 209040_s_at | 0.278 | TATCGGCCTAATCTTAGCCCTGAAG | 366 |
| FN1 | 214701_s_at | 0.278 | CGTCGACCAATGCCAGGATTCAGAG | 367 |
| COL1A1 | 202310_s_at | 0.277 | CAGACAAGCAACCCAAACTGAACCC | 368 |
| FOS | 209189_at | 0.277 | TCTCCTTAGTCTTCTCATAGCATTA | 369 |
| CYLD | 222142_at | 0.277 | TTCACCACCTGACTTTGAATTTGTC | 370 |
| ADAMTS1 | 222162_s_at | 0.276 | TGGTGAATGTCTGTTCAGCTCTTCT | 371 |
| ALDOA | 200966_x_at | 0.275 | TTCCCTCGTGACAGTGGTGTGTGGT | 372 |
| GATA6 | 210002_at | 0.275 | GACATTCTTATGCTTCTTTTACAAC | 373 |
| YWHAB | 217717_s_at | 0.275 | CCCCCTTTCCTACAGCAATATGTTC | 374 |
| CIB1 | 201953_at | 0.274 | AGACATCAAGTCCCATTATGCCTTC | 375 |
| OPTN | 202074_s_at | 0.273 | GAACAAGTGACTCTGACCAGCAGGC | 376 |
| IFI16 | 208965_s_at | 0.273 | GACCTGGCTGAAACTCTTAAAAAAG | 377 |
| CFLAR | 211316_x_at | 0.273 | TCTTCTGGAGCCTGTGTACTGCGGA | 378 |
| PTGER4 | 204897_at | 0.272 | GATTCCCAAACGTGGTTACATTAGC | 379 |
| CCND1 | 208712_at | 0.272 | AGCAAGCTGCCGAACCAAAAGAATT | 380 |
| PDLIM5 | 212412_at | 0.271 | GGATTCTCAATGTATAAGTTGCCTT | 381 |
| HLA-F | 221875_x_at | 0.271 | AGCTACTCTCAGGCTGCAGTGTGAG | 382 |
| CYP1B1 | 202437_s_at | 0.27 | TTAGCCTTTACCTGTGAAGTGTTAC | 383 |
| SVIL | 202565_s_at | 0.27 | GTTTGCTCTGCATTTTTGATGATGG | 384 |
| RNASET2 | 217983_s_at | 0.27 | GGCCTGACGTAATTCACTCGTTTCC | 385 |
| TAGLN2 | 200916_at | 0.269 | CTGCCTACCATGGTCTGGGGCTTGA | 386 |
| IFI27 | 202411_at | 0.267 | TCTGCCATTGCGGCTGTCATTGCGA | 387 |
| FLII | 212025_s_at | 0.267 | ATCAGACCCTGACGAAGCCAAGTTG | 388 |
| STAT6 | 201331_s_at | 0.266 | GAACATGTGTCTATCTGCTTTTGCC | 389 |
| WWP2 | 204022_at | 0.266 | TCAGGGTGCAAGCTGCTGGTGAGGT | 390 |
| FLNC | 207876_s_at | 0.266 | CTGCGAGGCCAGGGAAGCCCTGAGT | 391 |
| PARP12 | 218543_s_at | 0.266 | TTGTCTCTGCAGTGATTCGGTTTCT | 392 |
| VPS13D | 220221_at | 0.266 | GACTGGCATTTTACTTCTGTGTTTT | 393 |
| IFITM2 | 201315_x_at | 0.265 | ATCATTGAGGCCAGGAGCTCTGCCC | 394 |
| CTSZ | 210042_s_at | 0.265 | ACTGGCTGCGAGTGTTCCTGAGAGT | 395 |
| C19orf10 | 221739_at | 0.265 | GTGAAGCTGAAGGGGCCTGTGTCCC | 396 |
| DAPK1 | 203139_at | 0.264 | TGATCAGTGTTGTTGCTCTAGGAAG | 397 |
| LOC101928189 | 209006_s_at | 0.264 | GTGCATGCTAGAATTTGGGACAGGC | 398 |
| RSRP1 | | | | |
| MYOF | 211864_s_at | 0.263 | CTCTACTCTTTGCCGAACTATTTGT | 399 |
| ATP2B4 | 212135_s_at | 0.263 | CGCCCTTTCACTTAGTTCAGTGCCA | 400 |
| AXL | 202686_s_at | 0.262 | ATTGGTCCAAGATTCCGGATCCTAA | 401 |
| MIR6787 | 202855_s_at | 0.262 | TGTTCGTGGTGAGCTACGCCAAGGA | 402 |
| SLC16A3 | | | | |
| LY96 | 206584_at | 0.262 | CAGTATTGGGTCTGCAACTCATCCG | 403 |
| FN1 | 212464_s_at | 0.262 | CTGTTCTGCTTCGAAGTATTCAATA | 404 |
| CREB3L1 | 213059_at | 0.261 | TTGTATTTTACAAATCTCCCTCTTC | 405 |
| TNFSF12- | 209499_x_at | 0.26 | GTGTCATAATTCCCCGGGCAAGGGC | 406 |
| TNFSF13 | | | | |
| TNFSF13 | | | | |
| POFUT2 | 209578_s_at | 0.26 | GTTGCATTTTCCAGGCTGAGAGCTG | 407 |
| WDR1 | 200609_s_at | 0.259 | GTTGCCTGTCAGTGTTTACAAACTA | 408 |
| SLC7A7 | 204588_s_at | 0.259 | TAAAAGGGCCCACAATGCTCCAATT | 409 |
| MICB | 206247_at | 0.259 | ACCGTTATGCATTACTCTGTGTCTA | 410 |
| GATA3 | 209604_s_at | 0.258 | GTGTCTGTGTTCCAACCACTGAATC | 411 |
| LRRFIP1 | 211452_x_at | 0.257 | GAGCTATGATTGCACACTGTACTCT | 412 |
| RNASET2 | 217984_at | 0.256 | TAGATCGTGGCCCTTCAATTTAGAA | 413 |
| ITM2A | 202746_at | 0.255 | GCAAGCTCTTGTCAAATTTTAGTTT | 414 |

### RNA Extraction and Biomarker Expression Measurement

Cell samples or tissue samples may be snap frozen in liquid nitrogen until processing. RNA may be extracted using, e.g., Trizol Reagent from Invitrogen following manufacturer's instructions, and detected directly or converted to cDNA for detection. RNA may be amplified using, e.g., MessageAmp kit from Ambion following manufacturer's instructions. Amplified RNA may be quantified using, e.g., HG-U133A or HG-U133_Plus2 GeneChip from Affymetrix Inc. and compatible apparatus e.g., GCS3000Dx from Affymetrix, using the manufacturer's instructions. Exemplary Affymetrix probes for detecting the genes in Table 1 and Table 2 are described in PCT Patent Publication No. WO 2019/219759 and U.S. Patent Publication No. US 2021/0222252, each of which is incorporated by reference in its entirety. The resulting biomarker expression measurements may be further analyzed as described herein. The procedures described can be implemented using, e.g., R software available from R-Project and supplemented with packages available from Bioconductor.

One or more of the biomarkers shown in Table 1 and/or Table 2 (e.g., SRSF7 and/or HLA-E) may be measured in a biological sample (e.g., a tumor sample) obtained from the cancer subject using, e.g., polymerase chain reaction (PCR), reverse transcriptase PCR (RT-PCR), quantitative real-time PCR (qPCR), an array (e.g., a microarray), a genechip, pyrosequencing, nanopore sequencing, sequencing by synthesis, sequencing by expansion, single molecule real time technology, sequencing by ligation, microfluidics, infrared fluorescence, next generation sequencing (e.g., RNA-Seq techniques), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), proteomic techniques (e.g., mass spectrometry or protein arrays), and combinations thereof. In a particular example, biomarkers for sensitivity or resistance to stenoparib or a pharmaceutically acceptable salt thereof or a hydrate thereof can be measured using any of the methods (e.g., microarray, PCR-based techniques, and/or sequencing) described in PCT Patent Publication No. WO 2019/219759 and U.S. Patent Publication No. US 2021/0222252, each of which is incorporated by reference in its entirety.

### Methods of Determining the Responsiveness of a Subject to Stenoparib or a Pharmaceutically Acceptable Salt Thereof or a Hydrate Thereof

The disclosure features diagnostic methods for the detection and screening of a subject with a cancer (e.g., a subject with cancer or a recurrence thereof) that may be responsive to stenoparib or a pharmaceutically acceptable salt thereof or a hydrate thereof using one or more of the biomarkers shown in Table 1 and/or Table 2 (e.g., SRSF7 and/or HLA-E). The methods of the disclosure may be used for predicting a subject's responsiveness to stenoparib or a pharmaceutically acceptable salt thereof or a hydrate thereof, and optionally, treating the cancer subject throughout the progression of cancer and/or in cases of recurrence (e.g., after a first line treatment, a second line treatment, and/or a third line treatment).

The disclosure provides individual biomarkers (e.g., SRSF7 or HLA-E) and sets of biomarkers (e.g., two or more of the biomarkers listed in Table 1 and/or Table 2), the expression levels of which, as detected in a biological sample (e.g., a tumor sample, such as a biopsy) obtained from the subject with cancer, are indicative of the responsiveness of the subject to stenoparib or a pharmaceutically acceptable salt thereof or a hydrate thereof. The biomarkers were identified using methods similar to those previously described in, e.g., Chen et al. (Mol. Cancer Ther. 11:34-33, 2012), Wang et al. (J. Nat. Cancer Inst. 105:1284-1291, 2013), Knudsen et al. (PLOS One, 9: e87415, 2014), and Buhl et al (PLOS One 13 (3): e0194609, 2018), each of which is incorporated by reference herein in their entirety. In particular, an algorithm based on growth inhibition values (GI50) of a cell line (e.g., NCI60 cells) is used. The cell line is subjected to treatment with stenoparib or a pharmaceutically acceptable salt thereof or a hydrate thereof and baseline gene expression is determined (e.g., by microarray analysis, RT-PCR, qPCR, or next generation sequencing). After normalization, genes with, e.g., a Pearson correlation coefficient greater than 0.25 or below-0.25 were classified as biomarkers of sensitivity or resistance, respectively. In particular, a correlation coefficient of 0.25 or greater was used as a statistically significant cut-off for establishing that the expression levels of, e.g., the genes shown in Table 1 and/or Table 2, correlate with the likelihood of cancer treatment sensitivity, such as sensitivity to stenoparib or a pharmaceutically acceptable salt thereof or a hydrate thereof, as described in van′t Veer et al. Nature 415 (6871): 530-536, 2002, hereby incorporated by reference.

Thus, after normalization, expression levels of one or more of the biomarkers shown in Table 1 and/or Table 2 in a sample (e.g., a tumor sample) from a subject that are above a cutoff value of the 50th percentile in a reference population with the same diagnosis as the subject, or greater (e.g., 60th percentile, 70th percentile, or 80th percentile, or greater), indicate sensitivity and/or resistance, respectively, of the subject to treatment with stenoparib or a pharmaceutically acceptable salt thereof or a hydrate thereof.

Additionally, the predicted responsiveness of a cancer subject may be determined from, e.g., the difference score, which is the difference between the mean score (e.g., the mean of the expression level) of the one or more biomarkers of sensitivity of Table 1 (e.g., SRSF7) and the mean score (e.g., mean of the expression level) of the one or more biomarkers of resistance of Table 2 (e.g., HLA-E). The difference score of the cancer subject can then be compared to the difference score in a cell (e.g., a cancer cell) or tissue (e.g., a tumor tissue) known to be sensitive or resistant to treatment with stenoparib or a pharmaceutically acceptable salt thereof or a hydrate thereof.

For example, the cancer subject may be determined to be responsive to treatment with stenoparib or a pharmaceutically acceptable salt thereof or a hydrate thereof if the difference score is above a cutoff value of about 0.3 (e.g., about 0.3, about 0.35, about 0.4, about 0.45, about 0.5, about 0.55, about 0.6, about 0.65, about 0.7, or more). Alternatively, the cancer subject may be determined to be non-responsive to treatment with stenoparib or a pharmaceutically acceptable salt thereof or a hydrate thereof if the difference score is below a cutoff value of about 0.3 (e.g., about 0.29, about 0.28, about 0.27, about 0.26, about 0.2, about 0.15, about 0.1, about 0.05, about 0.02, about 0.01, or less).

Additionally, the mean score (i.e., mean of the expression level) of the one or more biomarkers of sensitivity of Table 1 and/or the mean score (i.e., mean of the expression level) of the one or more biomarkers of resistance of Table 2 can be used to predict responsiveness of a cancer subject (e.g., a subject with cancer recurrence) to stenoparib or a pharmaceutically acceptable salt thereof or a hydrate thereof. After determining the mean score of the biomarker(s) in a sample (e.g., a tumor sample) from the cancer subject, the mean score of the biomarker(s) in the sample may be compared to the mean score of the biomarker(s) in a cell (e.g., a cancer cell) or tissue (e.g., a tumor tissue) known to be sensitive to treatment with stenoparib or a pharmaceutically acceptable salt thereof or a hydrate thereof.

For example, the cancer subject (e.g., a subject with cancer recurrence) may be determined to be responsive to treatment with stenoparib or a pharmaceutically acceptable salt thereof or a hydrate thereof if the mean score (i.e., mean of the expression level) of the one or more biomarkers of sensitivity of Table 1 is above a cutoff value of about 0.3 (e.g., about 0.3, about 0.35, about 0.4, about 0.45, about 0.5, about 0.55, about 0.6, about 0.65, about 0.7, or more). Alternatively, the cancer subject (e.g., a subject with cancer recurrence) may be determined to be non-responsive to treatment with stenoparib or a pharmaceutically acceptable salt thereof or a hydrate thereof if the mean score of the one or more biomarkers of sensitivity of Table 1 is below a cutoff value of about 0.3 (e.g., about 0.29, about 0.28, about 0.27, about 0.26, about 0.2, about 0.15, about 0.1, about 0.05, about 0.02, about 0.01, or less). Additionally, the cancer subject (e.g., a subject with cancer recurrence) may be determined to be non-responsive to treatment with stenoparib or a pharmaceutically acceptable salt thereof or a hydrate thereof if the mean score (i.e., mean of the expression level) of the one or more biomarkers of resistance of Table 2 is above a cutoff value of about 0.3 (e.g., about 0.3, about 0.35, about 0.4, about 0.45, about 0.5, about 0.55, about 0.6, about 0.65, about 0.7, or more). Alternatively, the cancer subject (e.g., a subject with cancer recurrence) may be determined to be responsive to treatment with stenoparib or a pharmaceutically acceptable salt thereof or a hydrate thereof if the mean score of the one or more biomarkers of resistance of Table 2 is below a cutoff value of about 0.3 (e.g., about 0.29, about 0.28, about 0.27, about 0.26, about 0.2, about 0.15, about 0.1, about 0.05, about 0.02, about 0.01, or less).

In a particular example, the level(s) of any of the biomarkers from Table 1 and/or Table 2, the difference score, and/or the mean score can be used to predict sensitivity or resistance to treatment of a cancer using stenoparib or a pharmaceutically acceptable salt thereof or a hydrate thereof using any of the methods (e.g., analytical methods) described in PCT Patent Publication No. WO 2019/219759A1 and U.S. Patent Publication No. US 2021/0222252A1, each of which is incorporated by reference in its entirety.

### DRP® Companion Diagnostic for Dovitinib

The methods described herein include administration of stenoparib and dovitinib combination therapy to a subject having a cancer who may have sensitivity or resistance to treatment including dovitinib or a pharmaceutically acceptable salt thereof or a hydrate thereof. The methods may include determining, prior to therapy, whether the subject is likely to respond to the therapy, e.g., using a DRP® companion diagnostic for dovitinib.

A cancer subject can be assessed for sensitivity or resistance to dovitinib or a pharmaceutically acceptable salt thereof or a hydrate thereof by detecting gene expression of a biomarker (e.g., one or more of the biomarkers of Table 3 and/or Table 4) in a biological sample obtained from the cancer subject (e.g., a subject with the cancer or a recurrence thereof). The biological sample can include, for example, cells, tissue (e.g., a tissue sample obtained by biopsy), blood, serum, plasma, urine, sputum, cerebrospinal fluid, lymph tissue or fluid, or pancreatic fluid. For example, the biological sample can be fresh frozen or formalin-fixed paraffin embedded (FFPE) tissue obtained from the subject, such as a tumor sample (e.g., a biopsy) from the tissue of interest (e.g., lymph nodes, thymus, spleen, bone marrow, breast, colorectal, pancreatic, cervical, prostate, bladder, lung, gastrointestinal, head, neck, or ovarian tissue).

Table 3 below lists biomarkers for sensitivity to treatment with to dovitinib or a pharmaceutically acceptable salt thereof or a hydrate thereof, which are included in the dovitinib-DRP® companion diagnostic. Table 4 below lists biomarkers for resistance to treatment with to dovitinib or a pharmaceutically acceptable salt thereof or a hydrate thereof, which are included in the dovitinib-DRP® companion diagnostic.

TABLE 3
Biomarkers of sensitivity to dovitinib. Dashes mean that
the Affymetrix probeset has not been mapped to a specific
gene. Affymetrix IDS refer to the array type HG-U133_Plus 2

| | Affymetrix | Cor- | | SEQ ID |
|---|---|---|---|---|
| Gene | ID | relation | Affymetrix Probe Sequence | NO: |
| DDIT4 | 202887_s_at | 0.563 | GGCAGCTATCTTACAGACGCATGAA | 415 |
| ZNF395 | 218149_s_at | 0.331 | GTCCAGCGAATGACGTCTGTGGCCA | 416 |
| IncRNA | 216246_at | 0.326 | AGTCAATGAGTCGCTTGTGAATTCT | 417 |
| LSM4 | 202736_s_at | 0.307 | GAGCTGCGACAACTGGATGAACATT | 418 |
| SEL1L3 | 212314_at | 0.301 | TATTTTGGTACCTGTGCTTGCCACA | 419 |
| FABP5 | 202345_s_at | 0.296 | CAACACAGTTTTCTTGTACCCTGGG | 420 |
| TRIM22 | 213293_s_at | 0.293 | TGAGGTCAAATTTTATCTTTTCACT | 421 |
| TPK1 | 221218_s_at | 0.293 | TCATTACTTTCTGCTTGACCGGAAG | 422 |
| SLC16A3 | 202856_s_at | 0.291 | GAGTGGATCTGCGGTGAAGCCAAGC | 423 |
| INSIG1 | 201626_at | 0.288 | CAATAAGTCTTTCTCTCCGAAACCG | 424 |
| HOXA10- | 214651_s_at | 0.284 | AATGCGGGCATTTAAGTCTGTCCAT | 425 |
| HOXA9 | | | | |
| HOXA9 | | | | |
| MIR196B | | | | |
| DPP4 | 203716_s_at | 0.281 | AAAGCCCTGGTCGATGTTGGAGTGG | 426 |
| LDLR | 202068_s_at | 0.277 | AGAATGATGTCCCCGTTGTATGTAT | 427 |
| HSPE1 | 205133_s_at | 0.275 | TAGAAAGTTTCTTCCACTCTTTGAC | 428 |
| VEGFA | 212171_x_at | 0.274 | GGAGACCTGGTTGTGTGTGTGTGAG | 429 |
| TMSB10 | 217733_s_at | 0.272 | AAGAGCCACCTGCAAGATGGACACG | 430 |
| SLC16A3 | 202855_s_at | 0.271 | TGTTCGTGGTGAGCTACGCCAAGGA | 431 |
| VEGFA | 210512_s_at | 0.271 | GAATTCTACATACTAAATCTCTCTC | 432 |
| CAV2 | 203323_at | 0.269 | GATGAGCAGACTTCTCGGAATTCAT | 433 |
| P4HA1 | 207543_s_at | 0.267 | AACTAGGACACTAACTGGGTTCCAT | 434 |
| APOL1 | 209546_s_at | 0.265 | GTCCCCTGCTTGAACACTGAAGGGC | 435 |
| CAV2 | 203324_s_at | 0.264 | AAATGTTGCTCTAATCAGATTGCTT | 436 |
| PTPRE | 221840_at | 0.263 | GACACCTGTGTTTCAGCATTTGGAG | 437 |
| DPYSL2 | 200762_at | 0.262 | GACCCATGAGTGGAGGAACTTTCAG | 438 |
| ANP32B | 201305_x_at | 0.262 | ATGACCTGCAGAAACAGAACTGTTC | 439 |
| STAT6 | 201331_s_at | 0.26 | GCTGGCACCTTAGTTGCATGACCAC | 440 |
| RPL27A | 203034_s_at | 0.259 | TCTGCCCAACTGTCAACCTTGACAA | 441 |
| SNORA3 | | | | |
| EPB41L2 | 201719_s_at | 0.258 | GTTATACCATTTAAAGCTGGCACCA | 442 |
| RPL38 | 202028_s_at | 0.255 | AAAGGTATCTGCTGCATCGAACTTT | 443 |
| RNU86 | 211073_x_at | 0.253 | TTAAGTTCATTGACACCACCTCCAA | 444 |
| RPL3 | | | | |
| SNORD83B | | | | |

TABLE 4
Biomarkers of resistance to dovitinib. Dashes mean that
the Affymetrix probeset has not been mapped to a specific
gene. Affymetrix IDs refer to the array type HG-U133_Plus_2.
All correlations are negative.

| | Affymetrix | Cor- | | SEQ ID |
|---|---|---|---|---|
| Gene ID | ID | relation | Probe sequence | no |
| SCAMP3 | 201771_at | −0.397 | CTGCTCTGGTGGTGCCGAAGGGCAA | 445 |
| BAG5 | 202985_s_at | −0.329 | GATTGACTGTCAGGCATGGCTTTGT | 446 |
| ABCF1 | 200045_at | −0.325 | CTGATTTACCCTACAGCTTCAGGCC | 447 |
| MARCH6 | 201736_s_at | −0.313 | TAGCTTCTGGTGTTGTTCCTTTACT | 448 |
| EMC3 | 217882_at | −0.312 | GAGCTACTCACATTAGATGCATCCT | 449 |
| UCP2 | 208998_at | −0.304 | GAAAGTTCAGCCAGAATCTTCGTCC | 450 |
| TUG1 | 212337_at | −0.295 | ATACAGCAGTTCGAAAGCCGCGTCC | 451 |
| CLPTM1 | 211136_s_at | −0.286 | TTCATCTACCTCTACCAACGGTGGA | 452 |
| IGFBP5 | 211958_at | −0.285 | AGGAGGCTCCGTTTTGCAAAGTGGA | 453 |
| MAGEA1 | 207325_x_at | −0.278 | CTCAGTAGTAGGTTTCTGTTCTATT | 454 |
| ATM | 208442_s_at | −0.278 | CATTACGGGTGTTGAAGGTGTCTTC | 455 |
| GATA3 | 209602_s_at | −0.278 | CTGCTAGTCTTAAGAACTGCTTTCT | 456 |
| SPDEF | 220192_x_at | −0.278 | GGAAAACGGGCAGTCTGCTCTGCTG | 457 |
| LDOC1 | 204454_at | −0.276 | GCCGTTCAGCCTGGTTAGTTTTCTA | 458 |
| HRAS | 212983_at | −0.276 | GACTGTCTTGAACATCCCAAATGCC | 459 |
| SRM | 201516_at | −0.275 | TCGCCCACCAACCAAGTGTTACAAG | 460 |
| ZNF331 | 219228_at | −0.274 | CGCTTTCCACAGTTTGTTACCTGCA | 461 |
| GATA3 | 209604_s_at | −0.272 | GGACAAACTGCCAGTTTTGTTTCCT | 462 |
| TOB1 | 202704_at | −0.271 | CTCTCAGATATGGCCTCTTACAGTA | 463 |
| APITD1 CORT | 213454_at | −0.271 | CTTAAGCAAAATACTCCCAGGTCTC | 464 |
| NDUFV1 | 208714_at | −0.27 | GCACAGCTGCGGTGATCGTCATGGA | 465 |
| BAG6 | 213318_s_at | −0.27 | GTGGTATGCCTGCCAAGAGACGCAA | 466 |
| CUL3 | 201371_s_at | −0.269 | TATTCTTGGACTGTACTCTTCGCAT | 467 |
| CCDC90A | 220094_s_at | −0.268 | GAAATCATTGTGTCTGCATTGGTCA | 468 |
| CERS2 | 222212_s_at | −0.262 | GTAGGGGTTACAATTCACATTCCTT | 469 |
| LOC100506032 | 201103_x_at | −0.26 | GGTCTAGGAGATCTGTCCCTTTTAG | 470 |
| NBPF10 | | | | |
| NBPF11 | | | | |
| NBPF12 | | | | |
| NBPF15 | | | | |
| NBPF16 | | | | |
| NBPF24 NBPF7 | | | | |
| NBPF8 NBPF9 | | | | |
| PPP1R11 | 201500_s_at | −0.258 | CCATACCACCACTGAGATCTCATTT | 471 |
| CKB | 200884_at | −0.254 | GAAGCGAGGCACAGGCGGTGTGGAC | 472 |

### RNA Extraction and Biomarker Expression Measurement

Cell samples or tissue samples may be snap frozen in liquid nitrogen until processing. RNA may be extracted using, e.g., Trizol Reagent from Invitrogen following manufacturer's instructions, and detected directly or converted to cDNA for detection. RNA may be amplified using, e.g., MessageAmp kit from Ambion following manufacturer's instructions. Amplified RNA may be quantified using, e.g., HG-U133A or HG-U133_Plus2 GeneChip from Affymetrix Inc. and compatible apparatus e.g., GCS3000Dx from Affymetrix, using the manufacturer's instructions. The Affymetrix array typically contains 11 probes (also known as a probe set) specific to each gene. In general, confidence in a prediction of responsiveness or non-responsiveness increases with an increase in the number of probes used in the analysis. Exemplary Affymetrix probes for detecting the genes in Table 3 and Table 4 are described in PCT Patent Publication No. WO 2020/254383 and U.S. Pat. No. 10,835,531, each of which is incorporated by reference in its entirety. The resulting biomarker expression measurements may be further analyzed as described herein. The procedures described can be implemented using, e.g., R software available from R-Project and supplemented with packages available from Bioconductor.

One or more of the biomarkers shown in Table 3 and/or Table 4 (e.g., DDIT4) and/or SCAMP3) may be measured in a biological sample (e.g., a tumor sample) obtained from the cancer subject (e.g., a subject with any of the cancer types described herein, such as a subject with recurrence of cancer) using, e.g., polymerase chain reaction (PCR), reverse transcriptase PCR (RT-PCR), quantitative real-time PCR (qPCR), an array (e.g., a microarray), a genechip, pyrosequencing, nanopore sequencing, sequencing by synthesis, sequencing by expansion, single molecule real time technology, sequencing by ligation, microfluidics, infrared fluorescence, next generation sequencing (e.g., RNA-Seq techniques), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), proteomic techniques (e.g., mass spectrometry or protein arrays), and combinations thereof. In a particular example, biomarkers for sensitivity or resistance to dovitinib or a pharmaceutically acceptable salt thereof or a hydrate thereof can be measured using any of the methods (e.g., microarray, PCR-based techniques, and/or sequencing) described in PCT Patent Publication No. WO 2020/254383 and U.S. Pat. No. 10,835,531, each of which is incorporated by reference in its entirety

### Methods of Determining the Responsiveness of a Subject to Dovitinib or a Pharmaceutically Acceptable Salt Thereof or a Hydrate Thereof

The disclosure features diagnostic methods for the detection and screening of cancer subjects (e.g., subjects with cancer or a recurrence thereof) that may be responsive to dovitinib or a pharmaceutically acceptable salt thereof or a hydrate thereof using one or more of the biomarkers shown in Table 3 and/or Table 4 (e.g., DDIT4) and/or SCAMP3). The methods of the disclosure may be used for predicting a subject's responsiveness to dovitinib or a pharmaceutically acceptable salt thereof or a hydrate thereof, and optionally, treating the cancer subject throughout the progression of cancer and/or in cases of recurrence (e.g., after a first line treatment, a second line treatment, and/or a third line treatment).

The disclosure provides individual biomarkers (e.g., DDIT4 or SCAMP3) and sets of biomarkers (e.g., two or more of the biomarkers listed in Table 3 and/or Table 4), the expression levels of which, as detected in a biological sample (e.g., a tumor sample, such as a biopsy) obtained from a cancer subject (e.g., a human with cancer), are indicative of responsiveness to dovitinib or a pharmaceutically acceptable salt thereof or a hydrate thereof. The biomarkers were identified using methods similar to those previously described in, e.g., Chen et al. (Mol. Cancer Ther. 11:34-33, 2012), Wang et al. (J. Nat. Cancer Inst. 105:1284-1291, 2013), and Knudsen et al. (PLOS One, 9: e87415, 2014), Buhl et al (PLOS One 13 (3): e0194609, 2018) each of which are incorporated by reference herein in their entirety. In particular, an algorithm based on growth inhibition values (GI50) of a cell line (e.g., NCI60 cells) is used. The cell line is subjected to treatment with dovitinib or a pharmaceutically acceptable salt thereof or a hydrate thereof and baseline gene expression is determined (e.g., by microarray analysis, RT-PCR, qPCR, or next generation sequencing). After normalization, genes with, e.g., a Pearson correlation coefficient greater than 0.25 or below-0.25 were classified as biomarkers of sensitivity or resistance, respectively. In particular, a correlation coefficient of 0.25 or greater was used as a statistically significant cut-off for establishing whether the expression levels of, e.g., the genes shown in Table 3 and/or Table 4, correlate with the likelihood of cancer treatment sensitivity, such as sensitivity to dovitinib or a pharmaceutically acceptable salt thereof or a hydrate thereof, as described in van′t Veer et al. Nature 415 (6871): 530-536, 2002, hereby incorporated by reference.

Thus, after normalization, expression levels of one or more of the biomarkers shown in Table 3 and/or Table 4 in a sample (e.g., a tumor sample) from a subject that are above a cutoff value of the 50th percentile in a reference population with the same diagnosis as the subject, or greater (e.g., 60th percentile, 70th percentile, or 80th percentile, or greater), indicate sensitivity and/or resistance, respectively, of the subject to treatment with dovitinib or a pharmaceutically acceptable salt thereof or a hydrate thereof.

Additionally, the predicted responsiveness of a cancer subject may be determined from, e.g., the difference score, which may be defined as the difference between the mean score (e.g., mean of the expression level) of the one or more biomarkers of sensitivity of Table 3 (e.g., DDIT4) and the mean score (e.g., mean of the expression level) of the one or more biomarkers of resistance of Table 4 (e.g., SCAMP3). The difference score of the cancer subject can then be compared to the difference score in a cell (e.g., a cancer cell) or tissue (e.g., a tumor tissue) known to be sensitive or resistant to treatment with dovitinib or a pharmaceutically acceptable salt thereof or a hydrate thereof.

For example, the cancer subject (e.g., a subject with cancer recurrence) may be determined to be responsive to treatment with dovitinib or a pharmaceutically acceptable salt thereof or a hydrate thereof if the difference score is compared to a score from a reference population (e.g., a score determined using a tumor sample(s) from subjects diagnosed with the same type(s) of tumor as the subject), in which an expression level at the 50th percentile of the reference population, or the 60th percentile, or the 70th percentile, or the 80th percentile, or the 90th percentile, or greater, indicates that the sample (or the subject from whom the sample was taken) is predicted to be responsive to treatment with dovitinib or a pharmaceutically acceptable salt thereof or a hydrate thereof. The confidence of the prediction increases as the percentile level increases (e.g., an expression level above the 90th percentile of a reference population indicates a greater likelihood of treatment responsiveness than an expression level at the 50th percentile). Conversely, an expression level in the tested sample of below the 50th percentile of the reference population indicates that the sample (or the subject from whom the sample was taken) is predicted to be non-responsive to treatment with dovitinib or a pharmaceutically acceptable salt thereof or a hydrate thereof.

Additionally, the mean score (i.e., mean of the expression level) of the one or more biomarkers of sensitivity of Table 3 and/or the mean score (i.e., mean of the expression level) of the one or more biomarkers of resistance of Table 4 can be used to predict responsiveness of a cancer subject (e.g., a subject with cancer recurrence) to dovitinib or a pharmaceutically acceptable salt thereof or a hydrate thereof. After determining the mean score of the biomarker(s) in a sample (e.g., a tumor sample) from the cancer subject, the mean score of the biomarker(s) in the sample may be compared to the mean score of the biomarker(s) in a cell (e.g., a cancer cell) or tissue (e.g., a tumor tissue) known to be sensitive to treatment with dovitinib or a pharmaceutically acceptable salt thereof or a hydrate thereof. If the mean score of the biomarker(s) in the sample from the cancer subject is substantially similar to the mean score of the biomarker(s) in the cell or tissue known to be sensitive to dovitinib or a pharmaceutically acceptable salt thereof or a hydrate thereof, then the cancer subject is predicted to be responsive to treatment with dovitinib or a pharmaceutically acceptable salt thereof or a hydrate thereof. Alternatively, if the mean score of the biomarker(s) in the sample from the cancer subject is substantially dissimilar to the mean score of the biomarker(s) in the cell or tissue known to be sensitive to dovitinib or a pharmaceutically acceptable salt thereof or a hydrate thereof, then the cancer subject is predicted to be non-responsive to treatment with dovitinib or a pharmaceutically acceptable salt thereof or a hydrate thereof.

For example, the cancer subject (e.g., a subject with cancer recurrence) may be determined to be responsive to treatment with dovitinib or a pharmaceutically acceptable salt thereof or a hydrate thereof if the mean score (i.e., mean of the expression level) of the one or more biomarkers of sensitivity of Table 3 is compared to a mean score from a reference population (e.g., a mean score determined using a tumor sample(s) from subjects diagnosed with the same type(s) of tumor as the subject), in which an expression level at the 50th percentile of the reference population, or the 60th percentile, or the 70th percentile, or the 80th percentile, or the 90th percentile, or greater, indicates that the sample (or the subject from whom the sample was taken) is predicted to be responsive to treatment with dovitinib or a pharmaceutically acceptable salt thereof or a hydrate thereof. Conversely, an expression level in the tested sample of below the 50th percentile of the reference population indicates that the sample (or the subject from whom the sample was taken) is predicted to be non-responsive to treatment with dovitinib or a pharmaceutically acceptable salt thereof or a hydrate thereof.

Alternatively, the cancer subject (e.g., a subject with cancer recurrence) may be determined to be non-responsive to treatment with dovitinib or a pharmaceutically acceptable salt thereof or a hydrate thereof if the mean score (i.e., mean of the expression level) of the one or more biomarkers of resistance of Table 4 is compared to a mean score from a reference population (e.g., a mean score determined using a tumor sample(s) from subjects diagnosed with the same type(s) of tumor as the subject), in which an expression level at the 50th percentile of the reference population, or the 60th percentile, or the 70th percentile, or the 80th percentile, or the 90th percentile, or greater, indicates that the sample (or the subject from whom the sample was taken) is predicted to be non-responsive to treatment with dovitinib or a pharmaceutically acceptable salt thereof or a hydrate thereof.

In a particular example, the level(s) of any of the biomarkers from Table 3 and/or Table 4 can be used to predict sensitivity or resistance to treatment of a cancer using dovitinib or a pharmaceutically acceptable salt thereof or a hydrate thereof using any of the methods (e.g., analytical methods) described in PCT Patent Publication No. WO 2020/254383 and U.S. Pat. No. 10,835,531, each of which is incorporated by reference in its entirety.

### Kits

A dosage form of stenoparib or a pharmaceutically acceptable salt thereof or a hydrate thereof and/or a dosage form of dovitinib or a pharmaceutically acceptable salt thereof or a hydrate thereof described herein may be provided in a kit. The kit may include (a) a compound used in a method described herein (e.g., stenoparib or a pharmaceutically acceptable salt thereof or a hydrate thereof or dovitinib or a pharmaceutically acceptable salt thereof or a hydrate thereof), and, optionally (b) informational material. Alternatively, the kit may include (a) stenoparib or a pharmaceutically acceptable salt thereof or a hydrate thereof, b) dovitinib or a pharmaceutically acceptable salt thereof or a hydrate thereof, and optionally, (c) informational material. The informational material can be descriptive, instructional, marketing, or other material that relates to the methods described herein and/or the use of the dosage form for the methods described herein. The informational material may contain methods described herein directed to a combination therapy of stenoparib and dovitinib or pharmaceutically acceptable salts thereof and/or use of the dosage form for the methods described herein directed to a combination therapy of stenoparib and dovitinib or pharmaceutically acceptable salts thereof.

The informational material of the kit is not limited in its form. In one instance, the informational material can include information about production of the compound(s), molecular weight of the compound, concentration, date of expiration, batch or production site information, and so forth. In one instance, the informational material relates to methods for administering the compound (e.g., stenoparib or a pharmaceutically acceptable salt thereof or a hydrate thereof and/or dovitinib or a pharmaceutically acceptable salt thereof or a hydrate thereof).

In addition to a dosage form described herein, the kit can include other components or ingredients, such as a container(s) of a solvent or buffer, a stabilizer, a preservative, a flavoring agent (e.g., a bitter antagonist or a sweetener), a fragrance, a dye or coloring agent, for example, to tint or color one or more components in the kit. Other component(s) can be included in the kit, but in different compositions or containers distinct from the composition or containing with stenoparib or a pharmaceutically acceptable salt thereof or a hydrate thereof and/or dovitinib or a pharmaceutically acceptable salt thereof or a hydrate thereof. In such instances, the kit can include instructions for admixing a compound described herein (e.g., stenoparib or a pharmaceutically acceptable salt thereof or a hydrate thereof) and a different compound described herein (e.g., dovitinib or a pharmaceutically acceptable salt thereof or a hydrate thereof), or for using a compound described herein (e.g., stenoparib or a pharmaceutically acceptable salt thereof or a hydrate thereof and/or dovitinib or a pharmaceutically acceptable salt thereof or a hydrate thereof) together with the other component(s).

In some instances, the components of the kit are stored under inert conditions (e.g., under nitrogen or another inert gas such as argon). In some instances, the components of the kit are stored under anhydrous conditions (e.g., with a desiccant). In some instances, the components are stored in a light blocking container, such as an amber vial.

A dosage form described herein can be provided in any form, e.g., liquid, dried or lyophilized form. The compounds described herein may be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a dosage form described herein. In some instances, the kit contains separate containers, dividers or compartments for the composition and informational material. For example, the composition can be contained in a bottle, vial, or syringe, and the informational material can be contained in a plastic sleeve or packet. In other instances, the separate elements of the kit are contained within a single, undivided container. For example, the dosage form is contained in a bottle, vial or syringe that has attached thereto the informational material in the form of a label. In some instances, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light-tight.

The kit optionally includes a device suitable for delivery of the dosage form, e.g., a syringe, pipette, forceps, measured spoon, swab (e.g., a cotton swab or wooden swab), or any such device.

The kits of the disclosure can include dosage forms of varying strengths to provide a subject with doses suitable for one or more of the initiation phase regimens, induction phase regimens, or maintenance phase regimens described herein. Alternatively, the kit can include a scored tablet to allow the user to administer divided doses, as needed.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1. Treatment of Cancer with BID Dosing Stenoparib Monotherapy

Stenoparib potently inhibits both PARP1 and PARP2 in cell-free assays in vitro. Human recombinant PARP1 and mouse recombinant PARP2 were inhibited with IC50 values of 1.0 and 1.2 nmol/L, respectively.

The effect of stenoparib on cell proliferation was assessed in three human cancer cell lines and IC50 values were determined. Stenoparib inhibited cell proliferation in an 8-day assay in the BRCA1 mutant human triple negative breast cancer cell line MDA-MB-436 with an IC₅₀ value of 0.219 μmol/L. In an 8-day cell proliferation assay in the SR human B cell lymphoma cell line, stenoparib inhibited proliferation with an IC50 value of 1.055 μmol/L. Stenoparib inhibited cell proliferation in a 6-day assay in the human acute myeloid leukemia MV-4-11-luc2/AcGFP cell line with an IC50 value of 1.105 μmol/L.

In an in vitro study, stenoparib inhibited proliferation of the murine lymphocytic leukemia parent cell line P388/S and the P-gp overexpressing P388/VMDRC.04 line to a similar extent, with IC50 values of 2.14 and 1.39 μmol/L, respectively. Addition of the P-gp antagonist verapamil had little effect on inhibition of proliferation by stenoparib in either cell line (IC50 values of 2.34 and 1.82 μmol/L in P388/S and P388/VMDRC.04, respectively). Taken together, these data indicate that P-gp overexpression has no effect on inhibition by stenoparib.

Anti-tumor activity was observed for stenoparib in the BRCA1 mutant MDA-MB-436 human breast cancer subcutaneous xenograft model. Consecutive 28-day administration of Stenoparib at doses of 30 and 100 mg/kg resulted in statistically significant tumor growth inhibition. Stenoparib treatment was well-tolerated at both doses; there was no significant body weight loss.

Stenoparib inhibited PARP activity in tumor tissue from MDA-MB-436 xenografts following a single dose at 30 or 100 mg/kg. PARP activity was assessed in tumor tissue from MDA-MB-436 xenografts following a single dose of Stenoparib at various time points from 1 to 36 hours post-dose. PARP activity was completely inhibited in all tumors at 1 and 6 hours post-dose. At 12 hours, low levels of PARP activity were observed in the 30 mg/kg treated animals but not the 100 mg/kg dosed animals, and at 24 hours PARP activity appeared restored to control levels in both groups of mice.

Administration of stenoparib at 100 mg/kg for 28 days caused statistically significant anticancer activity in the human acute myeloid leukemia (AML) MV-4-11-luc2/AcGFP survival model. Reduced disease burden, as measured by luciferase signal, was observed at 100 mg/kg stenoparib and this translated into a statistically significant survival benefit. No statistically significant reduction in disease burden by luciferase signal or survival benefit was recorded for stenoparib at 10 and 30 mg/kg doses. Stenoparib administration at 10, 30 or 100 mg/kg was well-tolerated without any significant body weight loss.

Stenoparib monotherapy was examined in a finalized Phase 1 study (An Open-Label, Multicenter, Phase ½ Study of Poly (ADP-Ribose) Polymerase (PARP) Inhibitor E7449 as Single Agent in Subjects With Advanced Solid Tumors or With B-cell Malignancies and in Combination With Temozolomide (TMZ) or With Carboplatin and Paclitaxel in Subjects With Advanced Solid Tumors [E7449-E044-101]), and featured a once daily dosing of stenoparib as a single 600 mg dose. None of the 35 subjects assessed had a best overall response (BOR) of CR based on investigator assessment using RECIST 1.1. The overall objective response rate (ORR; CR+PR) was 4.9% (n=2) with 1 subject each in the 600 mg (MTD) and 800 mg dose groups with a BOR of PR. Overall, a BOR of stable disease was observed in 13 subjects (31.7%). Both PR were predicted by DRP® for stenoparib after analyzing biopsies from 13 of the 41 subjects.

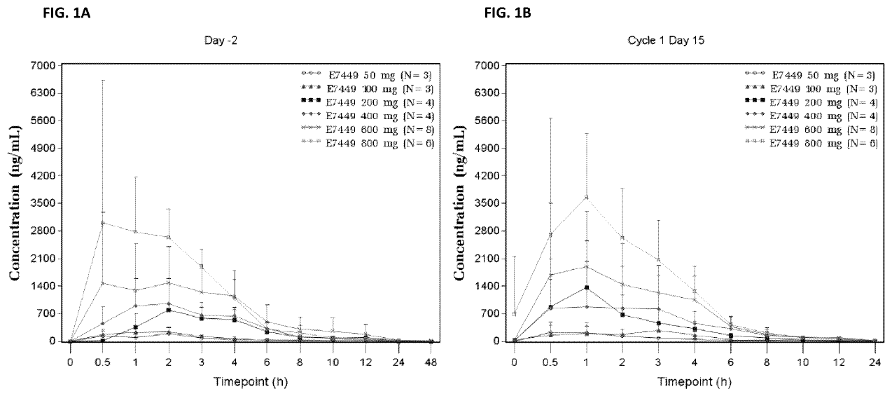

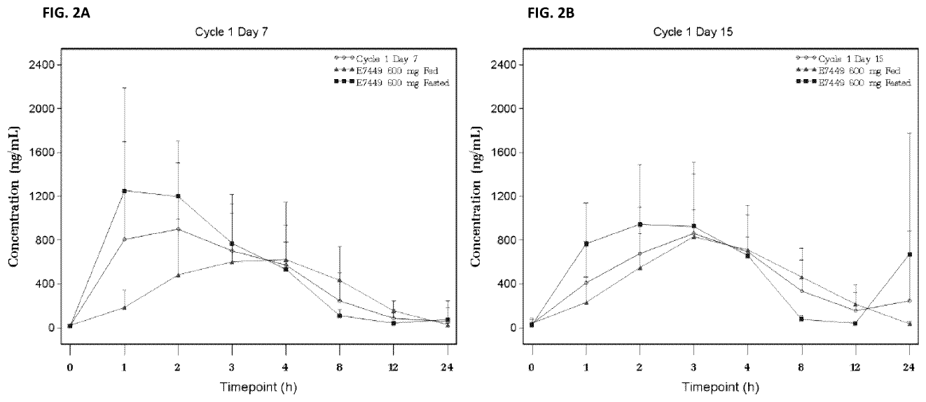

Stenoparib was well absorbed with tmax ranging from 0.5 to 4 hours across subjects and dose groups. The elimination half-life was approximately 8 hours with less than 1.5% of the administered dose recovered in urine. Following single or multiple oral doses between 50 mg and 800 mg a proportional absorption of stenoparib (e.g., in plasma) was found (FIG. 1A and FIG. 1B). At the 600 mg dose, food delayed stenoparib absorption and reduced Cₘₐₓ by 60%, and increased AUC by 10% (FIG. 2A and FIG. 2B) in plasma.

Dose dependent inhibition of PARP activity, as demonstrated by percent change in PAR levels, was observed. Maximal inhibition of PARP activity was observed at the MTD dose (600 mg) of single agent stenoparib. Evaluation of PAR levels at the MTD dose of stenoparib (600 mg) in the food effect cohort demonstrated that PAR levels show maximal decrease at 2 to 4 hours post-dose with up to 90% inhibition in PAR levels (from baseline) observed. Sustained PARP inhibition was observed with a 70% or greater decrease in PAR levels observed at 24 hours post-dose. Greater decrease in PAR levels was observed with increasing plasma concentration of stenoparib and with the maximal inhibition observed corresponding to the peak plasma concentration in measurements obtained at Day-2 and Cycle 1 Day 15. A greater decrease in PAR levels was observed with a corresponding higher Cₘₐₓ when stenoparib was administered without food than when administered with food.

The present study evaluates the safety and tolerability, antitumor activity, pharmacokinetics, and determine the maximum tolerated dose (MTD) of stenoparib monotherapy (at BID regimen) in subjects with advanced solid tumors.

### Dose Selection

In the above-mentioned Phase 1 trial, stenoparib, 600 mg, administered orally once daily, resulted in substantial and sustained PARP inhibition throughout the dosing interval and together with the efficacy observations supported the once daily dosing. However, the relative short half-life around 8 hours speaks to the possibility of increasing the daily dosing by changing from a QD dosing to a BID (i.e., twice per day) dosing.

Stenoparib, 600 mg, administered orally once daily was identified as the MTD in the phase I study, and was suggested as the RP2D for the subsequent phase II part of that study.

A breast cancer study was initiated in June 2018 and discontinued for futility after enrollment of 16 subjects. Twelve subjects were evaluable for response: Nine had PD and three subjects had SD.

The DLT from the phase one study was limited to fatigue and one case of anaphylactic reaction and no significant hematotoxicity or hepatotoxicity was observed though this would have been expected from the preclinical studies.

Safety data from the finalized Phase 1 study together with preliminary data from the Phase 2 study in metastatic breast cancer showed only dose-dependent drug related toxicity for fatigue and phototoxicity (average dose/sqm for each toxicity grade). At doses of 400 mg or higher fatigue was found in 30 subjects (grade 1-2: 25/42 (about 60%) and grade 3: 5/42 (about 12%)), and phototoxicity in 15 subjects (grade 1-2 14/42 (33%) and grade 3: 1/42 (2.6%)). Phototoxicity was in the Danish study primarily seen in 2 subjects treated during the hot summer period in 2018.

However, there does not seem to be dose related hematological and hepatological toxicity, that were the toxicities expected to be dose limiting from the preclinical studies and compared to other PARP inhibitors. Anemia was registered in only few subjects at doses of 400 mg and above 3/42 (7%) (all grade 2) and 4/42 had increased liver enzymes (grade 2: 1/42 (2.6%) and grade 3: 3/42 (7%)).

Gastrointestinal side-effects were registered in some subjects: nausea 14/42 (33%), vomiting 7/42 (16%) and diarrhea 13/42 (25%) all grade 1-2 apart from two cases of grade 3 diarrhea.

The efficacy was lower than expected with the only 2 subjects obtaining partial remission had received 600 and 800 mg respectively and furthermore had a relatively low body weight (61.4 and 63.7 kg). Thus, there is a risk that the recommended once daily dosing decided from the DLT in the phase 1 study may be too low. As discussed above, the half-life of stenoparib was discovered to be about 8 hours, so the initial dosing in the present study is a 200 mg am (morning) and 400 mg pm (evening), with a stepwise dose expansion of 200 mg daily to determine MTD using a 3+3 design.

### Objectives

The primary objective of this study is to determine the maximum tolerated dose (MTD) of stenoparib monotherapy given twice daily (BID) in subjects with advanced solid tumors.

The secondary objectives of this study are to evaluate the anti-tumor activity of stenoparib monotherapy, evaluate the pharmacokinetics (PK) of stenoparib monotherapy, and evaluate the safety and tolerability of stenoparib monotherapy.

### Study Outcome Measures

The MTD (e.g., of stenoparib) is defined as 1 dose level below the dose in which dose limiting toxicities (DLTs) are observed in ≥33% of the participants during the first 14 days of the main treatment period.

The secondary anti-tumor activity outcome measures in this study are:
To evaluate the objective response rate (ORR) of stenoparib monotherapy.
   ORR is defined as the proportion of subjects who achieve a Complete Response (CR) or Partial Response (PR) as assessed by RECIST v1.1.
To evaluate the duration of overall response (DOR) of stenoparib monotherapy.
   DOR is defined as the time in months from the first documented CR or PR per RECIST v. 1.1 to disease recurrence or disease progression (PD) whichever occurs first.
To evaluate progression free survival (PFS) of stenoparib monotherapy.
   PFS is defined as the time from study treatment initiation to either first observation of progressive disease or occurrence of death.
To evaluate overall survival (OS) of stenoparib monotherapy.
   OS is defined as the time from study treatment initiation to death from any cause or last day known to be alive.

### Determining MTD of Stenoparib

The determination of the maximum tolerated dose of stenoparib follows an accelerated titration method followed by a standard “3+3” design. The MTD is defined as one dose level (cohort) below the dose in which dose limiting toxicities (DLTs) were observed in ≥33% of the participants.

The calculation of the sample size for this trial is based on the traditional 3+3 dose escalation scheme which is conducted as follows:
Subjects are treated in cohorts of one (Cohort 1) or three (Cohorts 2-3) subjects, each receiving the same dose. For the assessment of a DLT, subjects are observed for 14 days.
In Cohort 1, if the one subject does not exhibit a DLT, the next cohort of three subjects will receive the next higher dose (Cohort 2). In Cohort 2, if none of the three subjects exhibits a DLT, the next cohort of three subjects will receive the next higher dose (Cohort 3).
Otherwise, if at least one subject of a cohort exhibits a DLT, a further cohort of three subjects is treated at the same dose level (cohort) without escalating the dose.
If exactly one out of the six subjects treated at this dose exhibits a DLT, the trial continues as planned at the next higher dose level (cohort).
If two or more subjects out of the six subjects treated at this dose exhibit a DLT, the dose escalation stops at that level and the next lower dose is considered as the MTD. When the escalation has stopped, additional subjects will be treated at the MTD to a total of six subjects.

Cohort escalation (e.g., the decision to progress from one cohort (dose level) to another) does not proceed until all of the following events have occurred:
(1) All study subjects in a given cohort (dose level) have been enrolled, and
(2) All such subjects have been followed for at least 14 days from the initiation of study treatment, and
(3) None of the DLTs outlined below have occurred, and further dose escalation is recommended
Hematological
   Grade 4 neutropenia [or Grade 3 neutropenia with fever (>38.5° C. in axilla)] for ≥7 days
   Grade 4 thrombocytopenia (Grade 3 thrombocytopenia with bleeding) lasting >7 days.
Other non-hematological toxicity
   Grade 3 fatigue, or a 2-point decline in Eastern Cooperative Oncology Group (ECOG) performance status that persists for >7 days.
   Nausea, vomiting or diarrhea that persists at Grade 3 or 4 despite maximal medical therapy.
   Any Grade 3 or higher non-hematological laboratory abnormalities that require hospitalization.

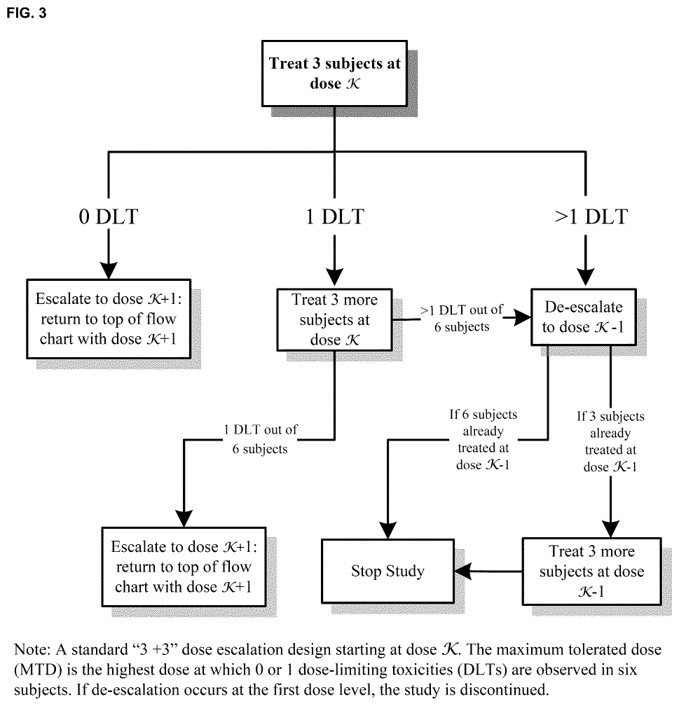

The MTD determination study design is illustrated in FIG. 3.

### Inclusion Criteria

Subjects are eligible for enrollment in the study if they meet the following criteria at time of Screening:
1. Age 18 years or older.
2. Histologically or cytological documented solid tumor.
3. Available tumor biopsy (most recent) for DRP® analysis.
4. Measurable disease by computed tomography (CT) scan or magnetic resonance imaging (MRI) if possible.
5. Performance status of Eastern Cooperative Oncology Group (ECOG)≤1.
6. Recovered to Grade <1 or baseline from prior surgery or from acute toxicity prior radiotherapy, or from treatment with cytotoxic, hormonal or biologic agents.
7. ≥2 weeks must have elapsed since any prior surgery or therapy with granulocyte colony-stimulating factor (G-CSF) and granulocyte-macrophage colony-stimulating factor (GM-CSF).
8. Subjects with intracranial disease must be on stable or decreased level of steroid therapy (e.g. dexamethasone) for at least 7 days prior to baseline MRI. Non-enzymatic inducing anti-epileptic drugs are allowed.
9. Adequate conditions as evidenced by the following clinical laboratory values:
   a. Absolute neutrophils count (ANC) ≥1500/mm3 (1.5×10³/mL)
   b. Hemoglobin >10.0 g/dL
   c. Platelets ≥100,000/mm3 (≥100×10⁹/L)
   d. Alanine aminotransferase (ALT) and aspartate aminotransferase (AST)≤3×ULN or ≤5×ULN in presence of liver metastases
   e. Serum bilirubin≤1.5 ULN
   f. Alkaline phosphatase≤2.5×ULN
   g. Creatinine≤1.5 ULN
   h. Blood urea nitrogen (BUN)≤2×ULN.
10. Life expectancy equal or longer than 3 months.
11. The subject is willing to provide written informed consent to participate in the study after reading the informed consent form and the information provided and has had the opportunity to discuss the study with the investigator or designee.
12. The subject is able to communicate satisfactorily with the investigator and to participate in, and comply with, the requirements of the study.
13. The subject is able to understand the nature of the study and any potential hazards associated with participating in it.
14. Negative pregnancy test for female subjects of childbearing potential. Women of childbearing potential (WOCBP) and Women of non-childbearing potential are eligible to participate. Both women of childbearing potential and women of non-childbearing potential should use an approved method of birth control and agree to continue to use this method for the duration of the study (and for 90 days after taking the last dose of study drug). All male subjects/partners must agree to consistently and correctly use a condom for the duration of the study and for 90 days after taking the study drug. In addition, subjects may not donate sperm for the duration of the study and for 90 days after taking study drug.

### Exclusion Criteria

Subjects meeting any of the following criteria at time of Screening are excluded from enrollment in the clinical study:
1. Concurrent chemotherapy, radiotherapy, hormonal therapy, or other investigational drug except non-disease related conditions (e.g., insulin for diabetes) during study period.
2. Other malignancy with exception of curative treated non-melanoma skin cancer or cervical carcinoma in situ within 5 years prior to entering the study.
3. Any active infection requiring parenteral or oral antibiotic treatment.
4. Known HIV positivity.
5. Known active hepatitis B or C.
6. Clinically significant (i.e., active) cardiovascular disease:
   a. Stroke within ≤6 months prior to day 1
   b. Transient ischemic attack (TIA) within $6 months prior to day 1
   c. Myocardial infarction within ≤6 months prior to day 1
   d. Unstable angina
   e. New York Heart Association (NYHA) Class II or greater congestive heart failure (CHF)
   f. Serious cardiac arrhythmia requiring medication.
7. Other medications or conditions, including surgery, that in the Investigator's opinion would contraindicate study participation for safety reasons or interfere with the interpretation of study results.
8. Inability to take oral medication, or malabsorption syndrome or any other uncontrolled gastrointestinal condition (e.g., nausea, diarrhea, or vomiting) that might impair the bioavailability of stenoparib and dovitinib.
9. Requiring immediate palliative treatment of any kind including surgery and/or radiotherapy.
10. Female subjects who are pregnant or breast-feeding (pregnancy test with a positive result before study entry).

### Study Treatment

The dose levels for stenoparib monotherapy BID dosing that are tested in this study are shown below in Table 5:

TABLE 5
Dosing for Stenoparib MTD Determination
for Stenoparib Monotherapy

| Dose Cohorts Dose Cohort | Stenoparib Monotherapy Dose (BID) |
|---|---|
| Cohort 1 | 600 mg (morning dose: 200 mg + |
| | evening dose: 400 mg) |
| Cohort 2 | 800 mg (morning dose: 400 mg + |
| | evening dose: 400 mg) |
| Cohort 3 | 1000 mg (morning dose: 400 mg + |
| | evening dose: 600 mg) |

On Day 1 of first treatment cycle (C1D1), subjects are administered stenoparib monotherapy as oral capsules taken twice daily. Each treatment cycle consists of 28 days.

Blood samples are collected for Stenoparib PK analysis at the following time points:
C1D1: prior to drug administration (morning dose), 1 h (±15 min), 3h (±15 min), 4h (±15 min), 6h (±15 min), 12h (±1 h, the sample should be taken before the evening dose)
C1D2: prior to drug administration (morning dose)
C1D7: prior to drug administration (morning dose)
C1D15: prior to drug administration (morning dose).
CXD1: prior to drug administration (morning dose) for the first day of all subsequent treatment cycles

Once the MTD has been determined, subjects enrolled in lower dose cohorts are allowed to escalate the dose to the MTD, if acceptable per the Investigator's discretion. Subjects are eligible for continuing treatment in absence of progressive disease or unacceptable toxicity or withdrawal of consent.

### Treatment Phase

Each treatment cycle is 28 days. Treatment consists of one or more treatment cycles, i.e., dosing cycles. A subject is considered to have completed the treatment phase once they complete the end of treatment (EOT) visit conducted 30 (+7) days after the last treatment visit.

A twice daily dose of stenoparib will be administered as hard gelatin capsules, independent of food intake in a 28-day cycle. The capsules are to be administered twice a day about the same time each treatment day.

### Dose Modification for Stenoparib

The dose level of stenoparib can be modified based on observed toxicity. Details of dose modification are summarized below in Table 6:

TABLE 6
Stenoparib Dose Modification

| | | Approximate dose |
|---|---|---|
| Stenoparib-Related Toxicity^{a} | During Therapy | Adjustment^{b} |

Grade 1

| | | |
|---|---|---|
| All occurrences | Continue treatment | Maintain dose level |

Grade 2

| | | |
|---|---|---|
| 1^{st} occurrence | Interrupt b until resolved to | Maintain dose level |
| | Grade 0-1 or baseline | |

| 2^{nd} occurrence (same toxicity or new toxicity) | 1^{st} dose reduction of stenoparib with 200 mg |
|---|---|
| | primarily, of the morning dose, secondarily, the |
| | evening dose |
| 3^{rd} occurrence (same toxicity or new toxicity) | 2^{nd} dose reduction of stenoparib with another |
| | 200 mg primarily, of the morning dose, |
| | secondarily, the evening dose |
| 4^{th} occurrence (same toxicity or new toxicity) | Discuss with sponsor |

Grade 3

| 1^{st} occurrence | Interrupt^{b} until resolved to | | 1^{st} dose reduction of stenoparib |
|---|---|---|---|
| | Grade 0-1 or baseline | | to with 200 mg primarily, of the |
| | | | morning dose, secondarily, the |
| | | | evening dose |
| 2^{nd} occurrence (same toxicity or new toxicity) | | 2nd dose reduction of stenoparib with another | |
| | | 200 mg primarily, of the morning dose, | |
| | | secondarily, the evening dose | |
| 3^{rd} occurrence (same toxicity or | Discontinue treatment | | Not Applicable |
| new toxicity) | | | |

Grade 4^{c}

| 1st occurrence | Interrupt until resolved to | 1^{st} dose reduction of stenoparib |
|---|---|---|
| | Grade 0-1 or baseline | with 400 mg primarily, of the |
| | | morning dose, secondarily, the |
| | | evening dose |
| 2nd occurrence (same toxicity | Discontinue treatment | Discuss with sponsor |
| or new toxicity) | | |

For grading see Common Toxicity Criteria for Adverse Events (CTCAE v5.0; cancer.gov, Nov. 27, 2017)^{a}: Excluding alopecia and nausea, vomiting and diarrhea not receiving adequate treatment. ^{b}: A delay of stenoparib for more than 21 days due to any toxicity will require a discussion with the sponsor before treatment can be resumed. ^{c}: Excluding laboratory abnormalities judged to be non-life-threatening, in which case manage as Grade 3.

### Example 2. Treatment of Cancer with BID Dosing Stenoparib and QD Dosing Dovitinib

This study evaluates safety and tolerability, antitumor activity, and pharmacokinetics of stenoparib (MTD) and dovitinib as combination therapy and determines the MTD of dovitinib when given in combination with the MTD of stenoparib determined in Example 1.

### Objectives

The primary objective of this study is to determine the MTD of dovitinib given in combination with stenoparib (MTD) in subjects with advanced solid tumors.

The secondary objectives of this study are to evaluate the anti-tumor activity of stenoparib in combination with dovitinib, evaluate the pharmacokinetics (PK) of stenoparib in combination with dovitinib, and evaluate the safety and tolerability of stenoparib in combination with dovitinib.

### Study Outcome Measures

The MTD (e.g., of stenoparib) is defined as 1 dose level below the dose in which dose limiting toxicities (DLTs) are observed in ≥33% of the participants during the first 14 days of the main treatment period.

The secondary anti-tumor activity outcome measures in this study are:
To evaluate the objective response rate (ORR) of stenoparib in combination with dovitinib.
   ORR is defined as the proportion of subjects who achieve a Complete Response (CR) or Partial Response (PR) as assessed by RECIST v1.1.
To evaluate the duration of overall response (DOR) of stenoparib in combination with dovitinib.
   DOR is defined as the time in months from the first documented CR or PR per RECIST v. 1.1 to disease recurrence or disease progression (PD) whichever occurs first.
To evaluate progression free survival (PFS) of stenoparib in combination with dovitinib.
   PFS is defined as the time from study treatment initiation to either first observation of progressive disease or occurrence of death.
To evaluate overall survival (OS) of stenoparib in combination with dovitinib.
   OS is defined as the time from study treatment initiation to death from any cause or last day known to be alive.

### Determining MTD of Dovitinib

The determination of the maximum tolerated dose of dovitinib follows the accelerated titration method followed by a standard “3+3” design described in Example 1 for stenoparib. The MTD is defined as one dose level (cohort) below the dose in which dose limiting toxicities (DLTs) were observed in ≥33% of the participants.

### Inclusion and Exclusion Criteria

This study shares the same Inclusion and Exclusion Criteria as described above in Example 1.

### Study Treatment

The dose levels for dovitinib dosing for combination treatment with stenoparib that are tested in this study are shown below in Table 7:

TABLE 7
Dosing for Dovitinib MTD Determination for Stenoparib
and Dovitinib Combination Therapy

| | Combination Stenoparib and |
|---|---|
| Dose Cohorts Dose Cohort | Dovitinib Dosing |
| Cohort 1 | Stenoparib (MTD) + 300 mg dovitinib |
| Cohort 2 | Stenoparib (MTD) + 400 mg dovitinib |
| Cohort 3 | Stenoparib (MTD) + 500 mg dovitinib |

Dovitinib will be administered once daily (morning) on a 5 days on and 2 days off schedule. In a 28 day cycle, dovitinib will be administered Cycle (C) 1 Day (D) 1 (C1D1)-C1D5, C1D8-C1D12, C1D15-C1D19, and C1D22-C1D26.

Dovitinib (TKI258), a pan-TKI, is an inhibitor of type III-V receptor tyrosine kinases (RTKs) that mediate both endothelial and tumor cell proliferation and survival. Dovitinib is formulated as a film-coated tablet of 100 mg strength.

Blood samples will be collected for stenoparib and dovitinib PK analysis at the following time points (in relation to stenoparib dosing):
C1D1: prior to drug administration (morning dose), 0.5h (±15 min), 1h (±15 min), 2h (±15 min), 3h (±15 min), 4h (±15 min), 6h (±15 min), 8h (±15 min), 10h (±1 h), 12h (±1 h, to be taken before the evening dose)
C1D2: prior to drug administration (morning dose)
C1D7: prior to drug administration (morning dose)
C1D15: prior to drug administration (morning dose), 0.5h (+15 min), 1h (+15 min), 2h (+15 min), 3h (+15 min), 4h (+15 min), 6h (+15 min), 8h (+15 min), 10h (+1 h), 12h (+1 h, to be taken before the evening dose)
CXD1: prior to drug administration (morning dose) for the first day of all subsequent treatment cycles

Once the MTD has been determined, subjects enrolled in lower dose cohorts will be allowed to escalate the dose to the MTD, if acceptable per the Investigator's discretion. Subjects will be eligible for continuing treatment in absence of progressive disease or unacceptable toxicity or withdrawal of consent.

### Treatment Phase

Each treatment cycle is 28 days. Treatment consists of one or more treatment cycles, i.e., dosing cycles. A subject is considered to have completed the treatment phase once they complete the EOT visit conducted 30 (+7) days after the last treatment visit.

A twice daily dose of stenoparib will be administered as hard gelatin capsules, independent of food intake in a 28 days cycle. Dovitinib tablets are supplied as 100 mg film-coated tablets in a blister pack. The capsules are to be administered twice a day about the same time each treatment day. The morning dose of stenoparib is administered at the same time as dovitinib.

Dovitinib will be administered once daily (morning) on a 5 days on and 2 days off schedule independent of food intake at the same time as the stenoparib morning dose. In a 28 day cycle, dovitinib will be administered C1D1-C1D5, C1D8-C1D12, C1D15-C1D19, and C1D22-C1D26.

### Dose Modification

Dose modification for dovitinib treatment may be allowable according to Table 8 below.

TABLE 8
Dose modification rules for dovitinib

| | |
|---|---|
| Dovitinib-Related Toxicity intensity^{a} | Dose modification^{b, c} |

Cardiovascular

| | |
|---|---|
| Hypertension | Treatment-emergent hypertension should be treated |
| | as per standard practice. |
| Systolic blood pressure (SBP) <160 mmHg | Maintain dose level |
| and/or diastolic blood pressure (DBP) <100 | |
| mm Hg, with or without anti-hypertensive | |
| medication | |
| ≥160 mmHg and/or DBP ≥100 mm Hg, with or | Delay the dovitinib treatment and initiate/intensify |
| without anti- hypertensive medication, but not | antihypertensive therapy. |
| immediately life-threatening | Dovitinib may be restarted in conjunction with |
| | standard anti- hypertensive medication if BP is |
| | controlled (i.e., BP <160/100 mmHg). |
| | Once BP is controlled after suspending dovitinib, |
| | maintain dose level or reduce one dose level at the |
| | discretion of the healthcare provider. |
| Life-threatening consequences (e.g., malignant | Discontinue dovitinib treatment permanently. |
| hypertension, transient or permanent | |
| neurologic deficit, hypertensive crisis); urgent | |
| intervention indicated | |

Left Ventricular Systolic Dysfunction

| | |
|---|---|
| Left ventricular ejection fraction (LVEF) ≥50%/ | Maintain dose level |
| lower limit of normal (LLN) | |
| (echocardiogram (ECHO)), or ≥45%/LLN | |
| (multigated acquisition (MUGA) scan) | |
| LVEF <50%/LLN (ECHO), or <45%/LLN | Delay dovitinib treatment until resolved to ≥50%/ |
| (MUGA), or | LLN (ECHO) or ≥45%/LLN (MUGA), then |
| Symptomatic due to drop in ejection fraction | reduce one dose level. |
| responsive to intervention | |
| Refractory or poorly controlled heart failure due | Discontinue dovitinib treatment. |
| to drop in ejection fraction; intervention such as | |
| ventricular assist device, intravenous | |
| vasopressor support or heart transplant | |
| indicated | |

Other Cardiovascular

| | |
|---|---|
| Grade 1 | Maintain dose level |
| Grade 2 | Maintain dose level |
| | If asymptomatic decrease of LVEF by absolute |
| | value of 20% and to < LLN, delay dovitinib treatment |
| | until resolved to ≤ grade 1, then reduce one dose |
| | level. |
| Grade 3 | Delay dovitinib treatment until resolved to ≤ grade 1, |
| | then reduce one dose level. |
| Grade 4 | Discontinue study treatment |

Gastrointestinal

| | |
|---|---|
| Diarrhea | At the first sign of abdominal cramping, loose stools, |
| | or onset of diarrhea, it is recommended that the |
| | subject be treated according to institutional standard |
| | of care. |
| Grade 1 (despite maximal anti- diarrheal | Maintain dovitinib dose level |
| medication) | |
| Grade 2 (despite maximal anti- diarrheal | Delay dovitinib treatment, until resolved to ≤ grade |
| medication) | 1, then re- start at the current dose level. |
| | If diarrhea returns as ≥ grade 2, then suspend dose |
| | until resolved to ≤ grade 1, then reduce one dose |
| | level. |
| Grade 3/4 (despite maximal anti- diarrheal | Delay dovitinib treatment until resolved to ≤ grade 1, |
| medication) | then reduce one dose level. |
| Nausea | Suspend dose for CTCAE grade 2-3 nausea only if |
| | it could not be controlled despite the use of standard |
| | anti-emetics. |
| Grade 1 (despite standard anti- emetics) | Maintain dovitinib dose level |
| Grade 2 (despite standard anti- emetics) | Delay dovitinib treatment, until resolved to ≤ grade |
| Grade 3 (despite standard anti- emetics) | 1, and then re-start at the current dose level. |
| | If nausea returns as > grade 2, then suspend dose |
| | until resolved to ≤ grade 1, then reduce one dose |
| | level. |
| | Delay dovitinib treatment until resolved to ≤ grade 1, |
| | then reduce one dose level. |
| Vomiting | Suspend dose for CTCAE grade 2-4 vomiting only if |
| | it cannot be controlled despite the use of standard |
| | anti-emetics. |
| Grade 1 (despite standard anti- emetics) | Maintain dovitinib dose level |
| Grade 2 (despite standard anti- emetics) | Delay dovitinib treatment, until resolved to ≤ grade |
| | 1, and then re-start at the current dose level. |
| | If nausea returns as > grade 2, then suspend dose |
| | until resolved to ≤ grade 1, then reduce one dose |
| | level. |
| Grade 3/4 (despite standard anti- emetics) | Delay study treatment until resolved to ≤ grade 1, |
| | then reduce 1 dose level. |

Hematologic

| | |
|---|---|
| Febrile neutropenia (Grade 3 or 4) | Delay dovitinib treatment until resolved, then reduce |
| (Per protocol) fever of unknown origin without | one dose level. |
| clinically or microbiologically documented | |
| infection (absolute neutrophil count (ANC) | |
| 1.0 × 10⁹/L, fever ≥38.5° C.) | |
| ≥Grade 3 anemia judged to be a hemolytic | Discontinue dovitinib treatment. |
| process secondary to study treatment | |
| ≥Grade 3 lymphopenia considered clinically | Requires dovitinib dose interruption until resolved to ≤ |
| significant | grade 1, then reduce dose level. |

Neutropenia/Neutrophil Count Decreased

| | |
|---|---|
| Grade 1 or 2 | Maintain dovitinib dose level. |
| Grade 3 | Delay dovitinib treatment until resolved to ≤ grade 2, |
| Grade 4 | then: |
| | If resolved by ≤7 days after suspending dovitinib, |
| | maintain dose level. |
| | If resolved by >7 days after suspending dovitinib, |
| | reduce one dose level. |

Thrombocytopenia/Platelet Count Decreased

| | |
|---|---|
| Grade 1 or 2 | Maintain dovitinib dose level. |
| Grade 3 | Delay dovitinib treatment until resolved to ≤ grade 1, |
| | then: |
| | If resolved by ≤7 days after suspending dovitinib, |
| | maintain dose level. |
| | If resolved by >7 days after suspending dovitinib, |
| | reduce one dose level. |
| Grade 4 | Delay dovitinib treatment until resolved to ≤ grade 1, |
| | then reduce one dose level. |

Hepatic

| | |
|---|---|
| ≥grade 3 alkaline phosphatase or GGT | Isolated values of ≥ grade 3 alkaline phosphatase or |
| | GGT values will NOT require dose interruption. |

Alanine Aminotransferase (ALT) or Aspartate Transaminase (AST)

| | |
|---|---|
| ≤3.0 × upper limit of normal (ULN) | Maintain dovitinib dose level with liver function tests |
| | (LFTs)^{e} monitored as per protocol. |
| >3.0 to ≤5.0 × ULN, | Maintain dovitinib dose level with weekly monitoring |
| without bilirubin elevation to >2.0 × ULN | of LFTs^{e}, or more frequently if clinically indicated, |
| | until resolved to ≤ grade 1 or baseline. |
| >5.0 × ULN | Delay dovitinib treatment until resolved to ≤ grade 1 |
| | or baseline, then reduce one dose level. |
| | Monitor LFTs^{e} weekly, or more frequently if clinically |
| | indicated, for 8 weeks; then, every 2 weeks for 4 |
| | weeks; then, every 4 weeks as per protocol. |
| | Following re-introduction of dovitinib, if ALT or AST |
| | elevations >3 × ULN recur as assessed on 2 |
| | separate measurements no more than 1 week |
| | apart, then study treatment should be permanently |
| | discontinued. |
| | If study treatment is permanently discontinued, then |
| | subjects should be monitored weekly (including |
| | LFTs^{e}), or more frequently if clinically indicated, until |
| | AST or ALT have resolved to ≤ grade 1 or |
| | stabilization (i.e., no CTCAE grade change over 4 |
| | weeks). |

Total Bilirubin

| | |
|---|---|
| ≤1.5 × ULN | Maintain dovitinib dose level with LFTs^{e} monitored |
| | as per protocol. |
| >1.5 to 3.0 × ULN | Maintain dovitinib dose level with LFTs^{e} monitored |
| with ALT or AST ≤3.0 × ULN | as per protocol. |
| >3.0 × ULN | Discontinue dovitinib treatment permanently. |
| | Subjects should be monitored weekly (including |
| | LFTs^{e}), or more frequently if clinically indicated, until |
| | total bilirubin has resolved to ≤ grade 1 or |
| | stabilization (i.e., no CTCAE grade change over 4 |
| | weeks). |
| | Note: If grade 3 or grade 4 hyperbilirubinemia is due |
| | to the indirect component only, and hemolysis as |
| | the etiology has been ruled out as per institutional |
| | guidelines (e.g., review of peripheral blood smear |
| | and haptoglobin determination), then reduce one |
| | dose level and continue dovitinib treatment at the |
| | discretion of the healthcare provider. |

ALT or AST, and Concurrent Bilirubin

| | |
|---|---|
| ALT or AST >3.0 × ULN and | Discontinue dovitinib treatment permanently. |
| Total Bilirubin >2.0 × ULN | Subjects should be monitored weekly (including |
| | LFTs^{e}), or more frequently if clinically indicated, until |
| | ALT/AST and total bilirubin have resolved to ≤ grade |
| | 1 or stabilization (i.e., no CTCAE grade change over |
| | 4 weeks). |

Renal Serum Creatinine

| | |
|---|---|
| Grade 1 or 2 | Maintain dose level |
| Grade 3 | Delay dovitinib treatment until resolved to ≤ grade 1 |
| | or baseline, then reduce one dose level |
| Grade 4 | Discontinue dovitinib treatment |

Amylase and/or Lipase Elevations

| | |
|---|---|
| Asymptomatic Grade 1 or 2 | Maintain dovitinib dose level |
| Asymptomatic Grade 3 or 4 | Subjects who develop grade 3 or 4 hyperlipasemia |
| | or hyperamylasemia without clinical or other |
| | evidence of pancreatitis may continue study |
| | medication without interruption. |

Pancreatitis

| | |
|---|---|
| Grade 2 | Maintain dovitinib dose level |
| Grade 3 or 4 | Discontinue dovitinib treatment |

Hand-Foot Syndrome

| | |
|---|---|
| Grade 1 or 2 | Maintain dovitinib dose level |
| Grade 3 | Delay dovitinib treatment until resolved to ≤ grade 1 |
| | then reduce 1 dose level. |
| Grade 4 | Discontinue dovitinib treatment |

Other Clinically Significant Adverse Events

| | |
|---|---|
| Grade 1 or 2 | Maintain dovitinib dose level |
| Grade 3 (except hyperlipidemia^{d}) | Delay dovitinib treatment until resolved to ≤ grade 1 |
| | or baseline, then maintain dose level or reduce one |
| | dose level at the discretion of the healthcare |
| | provider. |
| Grade 4 | Delay treatment until resolved to ≤ grade 1 or |
| | baseline, then reduce one dose level or discontinue |
| | dovitinib at the discretion of the healthcare provider. |

All dose modifications should be based on the worst preceding toxicity.
^{a}Common Terminology Criteria for Adverse Events (CTCAE) v4.03 (cancer.gov; Jun. 14, 2010)
^{b}Subjects are allowed two dose reductions: a dose reduction to 400 mg from 500 mg, and, if necessary, a dose reduction to 300 mg from 400 mg.
^{c}If a subject requires a dose interruption of >21 days starting from the first day a dose was missed, then the subject must be discontinued from the study. Subjects who discontinue the study for a study related adverse event including an abnormal laboratory value must be followed at least once a week for 30 days and subsequently at 30 days intervals until resolution or stabilization of the event, whichever comes first.
^{d}Grade 3 hyperlipidemia (hypercholesterolemia and/or hypertriglyceridemia) should be managed using standard therapies. Fluvastatin and Rosuvastatin may have potential of drug to drug interactions. If these statins are prescribed, close clinical monitoring including serum lipids is required since dovitinib could reduce the exposure of statins. Statins should be prescribed according to product label/data sheets for HMG-CoA reductase inhibitors. Treatment of hyperlipidemia should take into account the pre-treatment status and dietary habits. Blood tests to monitor hyperlipidemia must be taken in the fasting state. Grade 2 or higher hypercholesterolemia (>300 mg/dL or 7.75 mmol/L) or Grade 2 or higher hypertriglyceridemia (>2.5 × ULN) should be treated with fibrate, or appropriate lipid-lowering medication in addition to diet.
^{e}LFTs include albumin, ALT, AST, total bilirubin (fractionated [direct and indirect], if total bilirubin: 2.0 × ULN), alkaline phosphatase (fractionated [quantification of isoenzymes], if alkaline phosphatase ≥ Grade 2), and γ-glutamyl transferase (GGT).

### Example 3. Administration of Stenoparib BID to a Subject to Treat a Cancer

According to the methods disclosed herein, a subject, such as a human subject, with a solid tumor cancer (e.g., ovarian cancer) can be treated with a composition containing stenoparib or a pharmaceutically acceptable salt thereof or a hydrate thereof. The subject may be determined to be responsive to stenoparib or a pharmaceutically acceptable salt thereof or a hydrate thereof prior to treatment by using the Stenoparib DRP® described herein.

The subject may be administered stenoparib or a pharmaceutically acceptable salt thereof or a hydrate thereof orally twice a day, e.g., as a first dose and a second dose, according to a dosing regimen. The dosing regimen may include 2 to 8 dosing cycles. Each dosing cycle may be 28 days in length.

The subject may be administered a first dose of about 200 mg of stenoparib or a pharmaceutically acceptable salt thereof or a hydrate thereof (e.g., as one 200 mg hard gel capsule) and a second dose of about 400 mg of stenoparib or a pharmaceutically acceptable salt thereof or a hydrate thereof (e.g., as two 200 mg hard gel capsules) during each day of the dosing regimen; a first dose of about 400 mg of stenoparib or a pharmaceutically acceptable salt thereof or a hydrate thereof (e.g., as two 200 mg hard gel capsules) and a second dose of about 400 mg of stenoparib or a pharmaceutically acceptable salt thereof or a hydrate thereof (e.g., as two 200 mg hard gel capsules) during each day of the dosing regimen; or a first dose of about 400 mg of stenoparib or a pharmaceutically acceptable salt thereof or a hydrate thereof (e.g., as two 200 mg hard gel capsules) and a second dose of about 600 mg of stenoparib or a pharmaceutically acceptable salt thereof or a hydrate thereof (e.g., as three 200 mg hard gel capsules) during each day of the dosing regimen. The subject may be administered the second dose about 12 hours after administration of the first dose during each day of the dosing regimen. The subject may be administered the first dose and the second dose of stenoparib or the pharmaceutically acceptable salt thereof or the hydrate thereof at about the same time during each day of the dosing regimen.

### Example 4. Administration of Stenoparib BID and Dovitinib QD to a Subject to Treat a Cancer

According to the methods disclosed herein, a subject, such as a human subject, with a solid tumor cancer (e.g., prostate or ovarian cancer) can be treated with a first composition containing stenoparib or a pharmaceutically acceptable salt thereof or a hydrate thereof and a second composition containing dovitinib or a pharmaceutically acceptable salt thereof or a hydrate thereof. The subject may be determined to be responsive to stenoparib or a pharmaceutically acceptable salt thereof or a hydrate thereof and/or dovitinib or a pharmaceutically acceptable salt thereof or a hydrate thereof prior to treatment by using the Stenoparib DRPR and/or the Dovitinib DRP® described herein.

The subject may be administered stenoparib or a pharmaceutically acceptable salt thereof or a hydrate thereof orally twice a day, e.g., as a first dose and a second dose, and dovitinib or a pharmaceutically acceptable salt thereof or a hydrate thereof orally once a day, e.g., as a single dose, according to a dosing regimen. The subject may be administered dovitinib on a 5 days on and 2 days off schedule. The dosing regimen may include 2 to 8 dosing cycles. Each dosing cycle may be 28 days in length. For example, during a 28-day dosing cycle, the subject may be administered dovitinib or the pharmaceutically acceptable salt thereof or the hydrate thereof on Days 1-5, 8-12, 15-19, and 22-26, and not on Days 6, 7, 13, 14, 20, and 21.

The subject may be administered a first dose of about 200 mg of stenoparib or a pharmaceutically acceptable salt thereof or a hydrate thereof (e.g., as one 200 mg hard gel capsule) and a second dose of about 400 mg of stenoparib or a pharmaceutically acceptable salt thereof or a hydrate thereof (e.g., as two 200 mg hard gel capsules) during each day of the dosing regimen; a first dose of about 400 mg of stenoparib or a pharmaceutically acceptable salt thereof or a hydrate thereof (e.g., as two 200 mg hard gel capsules) and a second dose of about 400 mg of stenoparib or a pharmaceutically acceptable salt thereof or a hydrate thereof (e.g., as two 200 mg hard gel capsules) during each day of the dosing regimen; or a first dose of about 400 mg of stenoparib or a pharmaceutically acceptable salt thereof or a hydrate thereof (e.g., as two 200 mg hard gel capsules) and a second dose of about 600 mg of stenoparib or a pharmaceutically acceptable salt thereof or a hydrate thereof (e.g., as three 200 mg hard gel capsules) during each day of the dosing regimen. The subject may be administered the second dose about 12 hours after administration of the first dose during each day of the dosing regimen. The subject may be administered the first dose and the second dose of stenoparib or the pharmaceutically acceptable salt thereof or the hydrate thereof at about the same time during each day of the dosing regimen.

In addition, the subject may be administered a single dose of about 300 mg of dovitinib or a pharmaceutically acceptable salt thereof or a hydrate thereof (e.g., as three 100 mg film-coated tablets) during each day of the dosing regimen; a single dose of about 400 mg of dovitinib or a pharmaceutically acceptable salt thereof or a hydrate thereof (e.g., as four 100 mg film-coated tablets) during each day of the dosing regimen; or a single dose of about 500 mg of dovitinib or a pharmaceutically acceptable salt thereof or a hydrate thereof (e.g., as five 100 mg film-coated tablets) during each day of the dosing regimen. The subject may be administered the single dose of dovitinib or the pharmaceutically acceptable salt thereof or the hydrate thereof prior to administering the first dose of stenoparib or the pharmaceutically acceptable salt thereof or the hydrate thereof during each day of the dosing regimen that includes administration of dovitinib or the pharmaceutically acceptable salt thereof or the hydrate thereof.

### Example 5: Phase 2, Prospective Open Label, Single Arm Study to Investigate Anti-Tumor Effect and Tolerability of 2X-121 in Patients with Advanced Ovarian Cancer Selected by the 2X-121 DRP®

The primary objective of this study was to evaluate the anti-tumor efficacy of 2X-121 (i.e., stenoparib) as single agent therapy at 600 mg daily as QD (once daily) regimen compared to BID (twice-a-day) regimen in advanced ovarian cancer patients selected by the 2X-121 DRP®.

Patient population: Histologically or cytologically documented epithelial ovarian carcinoma including high-grade serous, endometrioid, clear cell, carcinosarcoma, undifferentiated, and mixed histological subtypes (independent of BRCA1 and homologous recombination (HR) deficiency (HRD) status) and with 2 or more previous chemotherapies/antibody therapies including treatment with other PARPi for advanced, refractory, or recurrent ovarian cancer. Patients were selected with DRP-2×121-Ovary (also known as stenoparib-DRP®) using the genes listed in Table 1 and 2.

### Results of First Interim Analysis:

22 patients were screened for BID dosing, and 23 patients had been screened for QD dosing, with 17 and 12 DRP-positive patients, respectively. Two patients consented but did not receive first dose and were not part of the Interim Analysis Population (IAP) or safety population. Table 5 below shows the baseline characteristics in the IAP.

TABLE 5
Baseline characteristics in the Interim Analysis
Population in terms of BRCA mutation status and
homologous recombination deficiency (HRD) status.

| | QD (N = 18) | BID (N = 10) |
|---|---|---|
| prior PARP treatment | | |
| Yes | 28% | 90% |
| No | 72% | 10% |
| tumor BRCA1/2 | | |
| Mut | 0% | 20% |
| WT | 28% | 0% |
| unknown | 72% | 80% |
| blood BRCA1/2 | | |
| Mut | 0% | 0% |
| WT | 33% | 70% |
| unknown | 67% | 30% |
| HRD status | | |
| positive | 0% | 20% |
| negative | 0% | 0% |
| unknown | 100% | 80% |

28 patients entered treatment: 11 from without DRP screening, 7 from QD (600 mg stenoparib daily) after DRP screening, and 10 from BID (200 mg stenoparib morning and 400 mg stenoparib evening) after DRP screening. The Interim Analysis Population (IAP) and safety population consisted of 28 patients (10 BID and 18 QD). Five patients received a first dose but dropped out before any tumor evaluation. Table 6 below shows the best overall tumor response in the IAP population.

TABLE 6
Tumor response (evaluated using RECIST 1.1)

| Tumor response | QD (N = 18 IAP) | BID (N = 10 IAP) |
|---|---|---|
| CR | 0 (0%) | 1 (10%) unconfirmed |
| PR | 0 (0%) | 0 (0%) |
| SD | 3 (17%) | 3 (30%) |
| SD > 16w | 1 (6%) | 1 (10%) |
| PD | 9 (50%) | 0 (0%) |
| Not evaluated | 5 (28%) | 5 (50%) |

Table 7 below shows the CA125 response evaluated according to Gynecologic Cancer Intergroup (GCIG) guidelines. Only patients with a baseline of CA125 twice the upper normal limit of 70 unit/ml who received at least one dose were included in IAP. Only patients who had completed 2 cycles were determined to be evaluable for CA125 response. Two QD patients and four BID patients were below 70 units/ml. The CA125 test was confirmed with a second measurement that was lower than or within 10% of the previous measurement.

TABLE 7
CA125 test response (GCIG)

| CA125 response | QD (N = 16 IAP) | BID (N = 6 IAP) |
|---|---|---|
| CR | 0 (0%) | 1 (17%) unconfirmed |
| PR | 0 (0%) | 0 (0%) |
| SD | 3 (19%) | 1 (17%) |

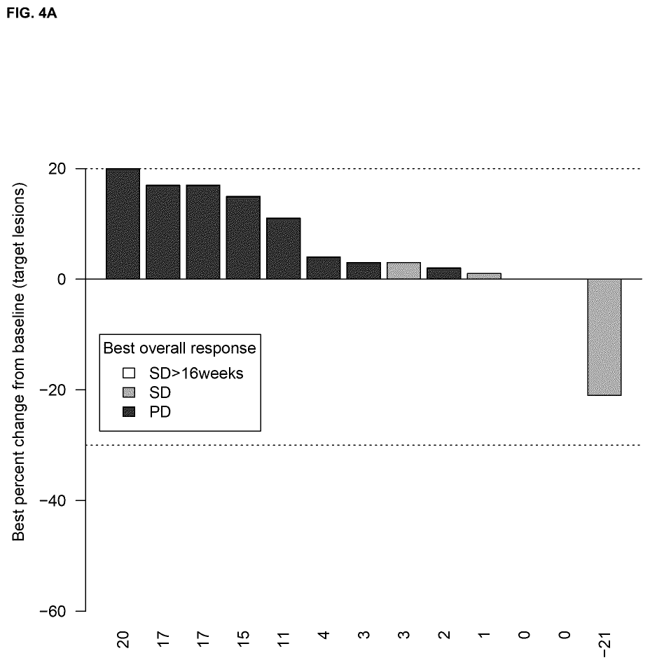

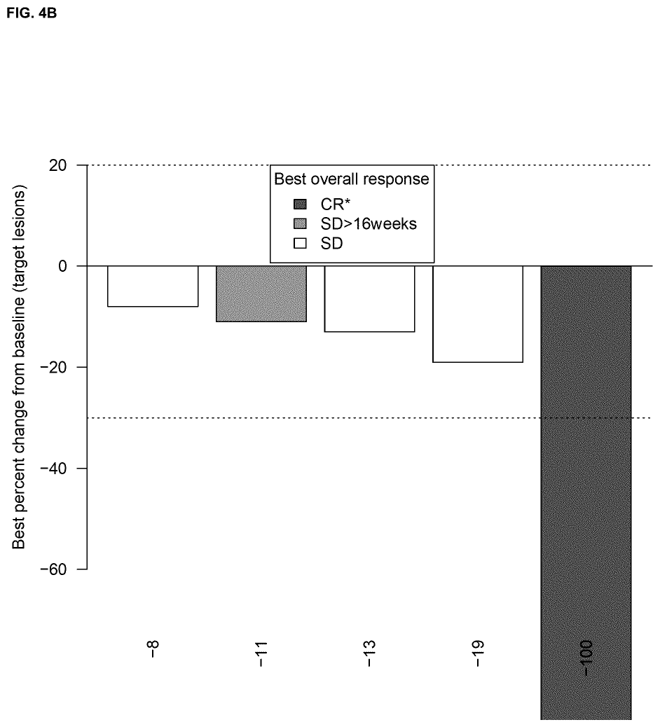

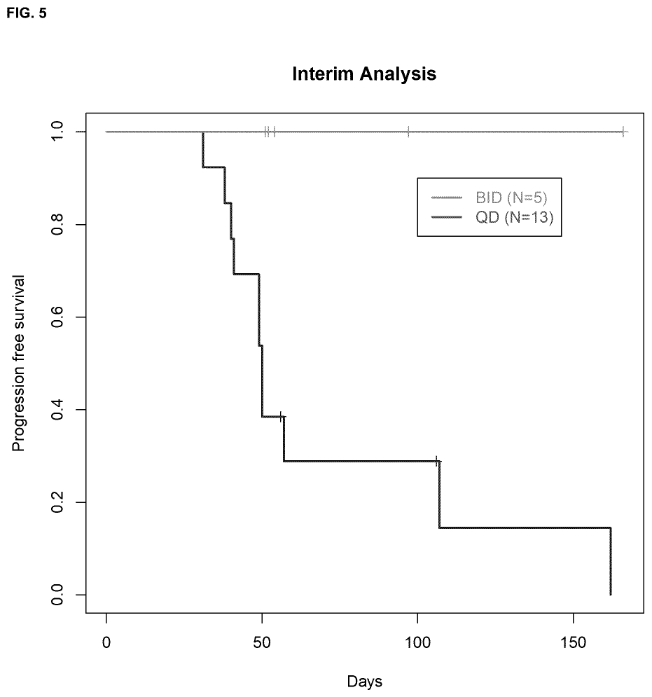

FIG. 4A and FIG. 4B show the best change in tumor diameter from baseline in the IAP QD population (FIG. 4A) and the IAP BID population (FIG. 4B). Progression free survival in the IAP population is shown in FIG. 5. The mean of best change in tumor diameter from baseline in the QD population (FIG. 4A) was 4% tumor growth. The CR in the BID pollution (FIG. 4B) was unconfirmed, and consisted of target lymph node lesions that all turned normal. This CR patient also had CA125 CR unconfirmed status. The mean of best change in tumor diameter from baseline in the BID population (FIG. 4B) was 29% tumor reduction. The median PFS was not reached in the BID population, and was 50 days (1.6 months) in the QD population.

### Analysis of DRP Scores

DRP scores correlation to best change in tumor diameter was assessed for the three cohorts:
US QD (no DRP selection): CC=0.30
UK QD (DRP selection): Pearson CC=0.41
US BID (DRP selection): Pearson CC=−0.49

To demonstrate a correlation between a DRP score and a reduction in tumor diameter, the correlation must be negative. Only the BID population exhibited such a negative correlation. These surprising results demonstrated that stenoparib DRP was surprisingly effective at predicting effectiveness of stenoparib BID treatment in patients with ovarian cancer.

In order to calculate a correlation coefficient for DRP scores and the RECIST best response category, the RECIST response categories were numerically encoded as follows: complete response (CR)=1, partial response (PR)=2, stable disease (SD)>16w=3, SD=4, disease progression (PD)=5. The resulting CC was obtained for the three for the three cohorts:
DFCI QD (no DRP selection): Pearson CC=0.37
UK QD (DRP selection): Pearson CC=0.93
US BID (DRP selection): Pearson CC=−0.60

For a correlation between a DRP score and a clinically beneficial response, the correlation must be negative. Only BID has such a negative correlation. These surprising results likewise demonstrated that stenoparib DRP was surprisingly effective at predicting effectiveness of stenoparib BID treatment in patients with ovarian cancer.

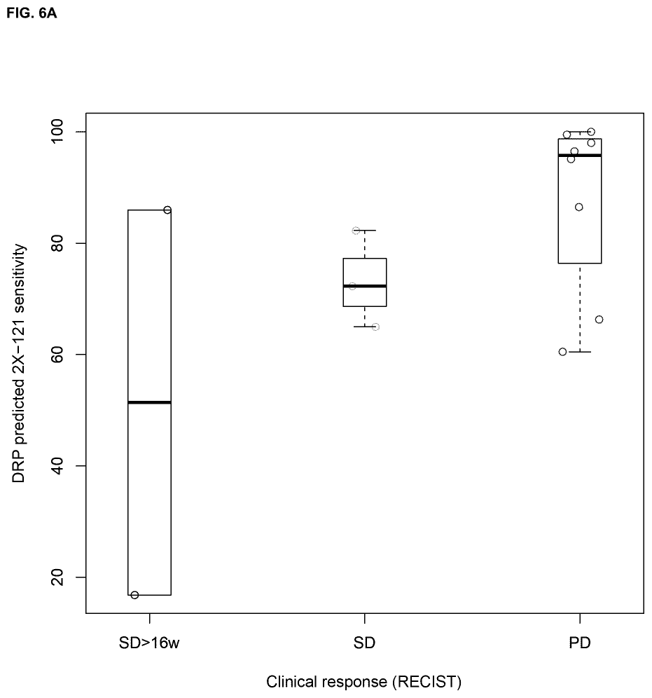

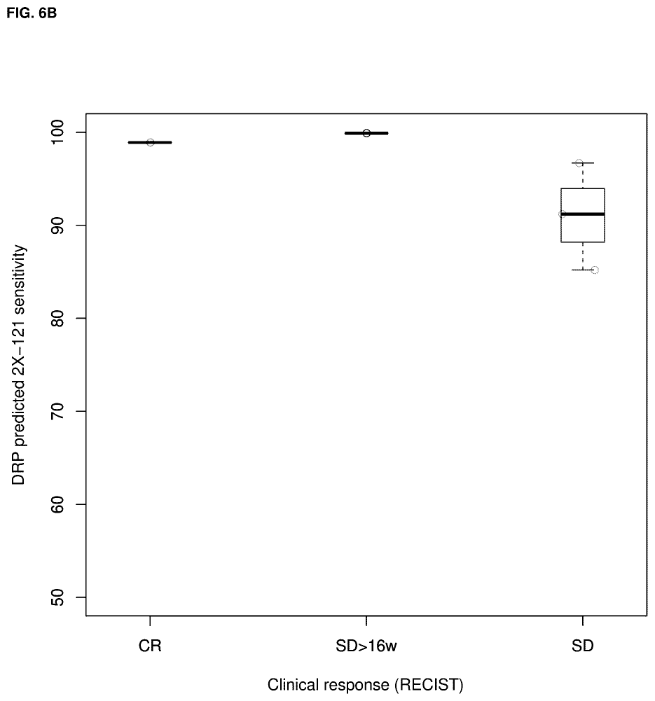

FIG. 6A and FIG. 6B show stripcharts of DRP scores in the individual RECIST categories. There was no apparent association between DRP score and RECIST category for QD dosed patients, as confirmed with a correlation analysis of QD scores. FIG. 6A shows a stripchart for QD dosed patients and FIG. 6B shows a stripchart for BID dosed patients. A correlation to RECIST category was found for BID dosed patients, as confirmed by the correlation analysis above. As is clear from the data, BID dosing was considerably more robust and effective than QD dosing.

### Safety

Adverse events and treatment-related adverse events in the safety population (N=28) are listed in Table 8 and Table 9 below. There was a bias in this evaluation in that most BID patients had been in treatment for a shorter duration than the QD patients, and thus had a longer period of time to experience adverse events. The Adverse events listed for the QUADRA trial in the niraparib FDA approval were included for comparison (see Moore et al., Lancet Oncol. 20:636-648, 2019).

TABLE 8
Adverse events in the safety population (N = 28)

| | Grade 1-4 | Grade 3-4 | Grade 5 |
|---|---|---|---|
| 600 mg QD (N = 18) | 100% | 61% | 6% |
| 600 mg BID (N = 10) | 70% | 20% | 0% |
| 300 mg QD niraparib (N = 463) | NA | NA | 2% |

TABLE 9
Treatment-related adverse events

| | Grade 1 | Grade 2 | Grade 3 | Grade 4 | Grade 5 |
|---|---|---|---|---|---|
| 600 mg QD | 94% | 83% | 33% | 0% | 0% |
| (N = 18) | | | | | |
| 600 mg BID | 70% | 60% | 20% | 0% | 0% |
| (N = 10) | | | | | |

| | | | | |
|---|---|---|---|---|
| 300 mg QD niraparib | 90% | 56% | 20% | <1% |
| (N = 463) | | | | |

Myelotoxicity that was observed for other PARP inhibitors (i.e., niraparib) is shown below in Table 10. In the published Phase I trial of 2X-121 (Plummer, Br J Cancer 2020. 123 (4): 525-533, 2020), there was no myelotoxicity among adverse events found in more than 5% of patients. Because myelotoxicity appeared to be a rare event for 2×-121, 14 patients with metastatic breast cancer that received 600 mg QD in a phase II trial were included in Table 10.

TABLE 10
Myelotoxicity that was observed for other PARP inhibitors

| | 2X-121 BID + QD (N = 42) | | Niraparib QD (N = 463) | |
|---|---|---|---|---|
| | Grade 1-4 | Grade 3-4 | Grade 1-4 | Grade 3-4 |
| Neutropenia | 2% | 0% | 20% | 13% |
| Anemia | 21% | 7% | 51% | 27% |
| Thrombocytopenia | 0% | 0% | 52% | 28% |

Dose modifications in patients with advanced ovarian cancer are shown and compared to niraparib in Table 11 below.

TABLE 11
Dose modifications in patients with advanced ovarian cancer

| | Reduction | Interruption | Discontinuation |
|---|---|---|---|
| 600 mg QD | 28% | 83% | 11% |
| (N = 18) | | | |
| 600 mg BID | 40% | 50% | 0% |
| (N = 9) | | | |
| 300 mg QD niraparib | 47% | 62% | 21% |
| (N = 463) | | | |

### CONCLUSION

BID dosing was more effective than QD dosing (FIG. 4A, FIG. 4B, and FIG. 5). Myelosuppression occurred less with 2X-121 (QD and BID) compared to approved PARP inhibitor niraparib. DRP scores showed correlation to clinical benefit in BID dosed patients, but not in QD dosed patients.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Although specific dosing regimens (e.g., including a pharmaceutical composition including stenoparib, or a pharmaceutically acceptable salt thereof, optionally with a pharmaceutical composition including dovitinib, or a pharmaceutically acceptable salt thereof) have been disclosed, a number of other embodiments and modifications may be made without departing from the spirit and scope of the invention. It should be apparent, however, to those skilled in the art that many more modifications besides those already described are possible without departing from the inventive concepts described herein. The inventive subject matter, therefore, is not to be restricted except in the spirit of the disclosure.

All patents, patent publications and publications mentioned herein are incorporated herein by reference in their entirety to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Other embodiments are within the claims.

1. A method of treating a cancer in a subject in need thereof comprising administering to the subject a dosing regimen comprising a first dose and a second dose of a composition comprising stenoparib or a pharmaceutically acceptable salt thereof or a hydrate thereof per day.

2. The method of claim 1, wherein:
(a) the dosing regimen comprises a dosing cycle comprising administration of the composition for 28 days±1 to 5 days;
(b) the dosing regimen comprises 2-100 dosing cycles;
(c) an amount of stenoparib or the pharmaceutically acceptable salt thereof or the hydrate thereof administered in the first dose is less than or equal to an amount of stenoparib or the pharmaceutically acceptable salt thereof or the hydrate thereof administered in the second dose;
(d) the first dose comprises between about 200 mg to about 400 mg of stenoparib or the pharmaceutically acceptable salt thereof or the hydrate thereof;
(e) the second dose comprises between about 400 mg to about 600 mg of stenoparib or the pharmaceutically acceptable salt thereof or the hydrate thereof; and/or
(f) the second dose is administered to the subject at least 11 to 13 hours after administration of the first dose.

3-4. (canceled)

5. The method of claim 2, wherein the dosing regimen comprises 2 to 8 dosing cycles.

6-8. (canceled)

9. The method of claim 1, wherein:
(a) the first dose comprises about 200 mg of stenoparib or the pharmaceutically acceptable salt thereof or the hydrate thereof and the second dose comprises about 400 mg of stenoparib or the pharmaceutically acceptable salt thereof or the hydrate thereof;
(b) the first dose comprises about 400 mg of stenoparib or the pharmaceutically acceptable salt thereof or the hydrate thereof and the second dose comprises about 400 mg of stenoparib or the pharmaceutically acceptable salt thereof or the hydrate thereof; or
(c) the first dose comprises about 400 mg of stenoparib or the pharmaceutically acceptable salt thereof or the hydrate thereof and the second dose comprises about 600 mg of stenoparib or the pharmaceutically acceptable salt thereof or the hydrate thereof.

10. (canceled)

11. The method of claim 1, wherein:
(a) the cancer:
(i) is a solid tumor cancer;
(ii) is locally advanced or metastatic;
(iii) comprises a BRCA mutation; and/or
(iv) is homologous recombination (HR) deficient; and/or
(b) the subject is non-responsive to at least one cancer therapy other than stenoparib or a pharmaceutically acceptable salt thereof or a hydrate thereof.

12. The method of claim 11, wherein the cancer is a solid tumor cancer, and wherein the cancer is an ovarian cancer, a breast cancer, a colon cancer, a pancreatic cancer, or a prostate cancer.

13-15. (canceled)

16. The method of claim 12, wherein the cancer is ovarian cancer, and wherein the ovarian cancer is an epithelial ovarian carcinoma.

17. The method of claim 16, wherein the epithelial ovarian carcinoma is a high-grade serous epithelial ovarian carcinoma, an endometrioid epithelial ovarian carcinoma, a clear cell epithelial ovarian carcinoma, an epithelial ovarian carcinosarcoma, an undifferentiated epithelial ovarian carcinoma, or an epithelial ovarian carcinoma of mixed histological subtypes.

18-19. (canceled)

20. The method of claim 1, wherein the subject is non-responsive to at least one cancer therapy other than stenoparib or a pharmaceutically acceptable salt thereof or a hydrate thereof, and wherein the at least one cancer therapy comprises a PARP inhibitor.

21. The method of claim 1, wherein;
(a) the subject is administered the composition orally; and/or
(b) the composition is formulated as a capsule or a tablet.

22. (canceled)

23. The method of claim 21, wherein the composition is formulated as a capsule, and wherein each capsule comprises about 50 mg, about 100 mg, or about 200 mg of stenoparib or the pharmaceutically acceptable salt thereof or the hydrate thereof.

24. The method of claim 1, wherein the method comprises, prior to administration of the composition, determining the responsiveness of the subject to treatment with stenoparib or the pharmaceutically acceptable salt thereof or the hydrate thereof by:
(a) determining the expression level of one or more biomarkers of sensitivity selected from the biomarkers of Table 1; and/or
(b) determining the expression level of one or more biomarkers of resistance selected from the biomarkers of Table 2.

25. A method of treating a cancer in a subject in need thereof comprising administering to the subject a dosing regimen comprising:
(a) administering a first dose and a second dose of a first composition comprising stenoparib or a pharmaceutically acceptable salt thereof or a hydrate thereof per day; and
(b) administering a dose of a second composition comprising dovitinib or a pharmaceutically acceptable salt thereof or a hydrate thereof.

26. The method of claim 25, wherein;
(a) the dosing regimen comprises a dosing cycle comprising 28 days+1 to 5 days;
(b) the dosing regimen comprises 2-100 dosing cycles:
(c) the method comprises administering the first and second doses of the first composition each day of the dosing cycle and administering the second composition on a 5 days on/2 days off schedule during the dosing cycle;
(d) an amount of stenoparib or the pharmaceutically acceptable salt thereof or the hydrate thereof administered in the first dose of the first composition is less than or equal to an amount of stenoparib or the pharmaceutically acceptable salt thereof or the hydrate thereof administered in the second dose of the first composition;
(e) the first dose of the first composition comprises between about 200 mg to about 400 mg of stenoparib or the pharmaceutically acceptable salt thereof or the hydrate thereof;
(f) the second dose of the first composition comprises between about 400 mg to about 600 mg of stenoparib or the pharmaceutically acceptable salt thereof or the hydrate thereof;
(g) the dose of the second composition comprises between about 300 mg to about 500 mg of dovitinib or the pharmaceutically acceptable salt thereof or the hydrate thereof; and/or
(h) the second dose is administered to the subject at least 11 to 13 hours after administration of the first dose.

27-28. (canceled)

29. The method of claim 26, wherein:
(a) the dosing regimen comprises 2-8 dosing cycles; and/or
(b) the dosing regimen comprises 28 days, and wherein the second composition is administered to the subject as a single dose on Days 1-5, Days 8-12, Days 15-19, and Days 22-26 of each said dosing cycle.

30-35. (canceled)

36. The method of claim 25, wherein:
(A)
(a) the first dose of the first composition comprises about 200 mg of stenoparib or the pharmaceutically acceptable salt thereof or the hydrate thereof and the second dose of the first composition comprises about 400 mg of stenoparib or the pharmaceutically acceptable salt thereof or the hydrate thereof;
(b) the first dose of the first composition comprises about 400 mg of stenoparib or the pharmaceutically acceptable salt thereof or the hydrate thereof and the second dose of the first composition comprises about 400 mg of stenoparib or the pharmaceutically acceptable salt thereof or the hydrate thereof; or
(c) the first dose of the first composition comprises about 400 mg of stenoparib or the pharmaceutically acceptable salt thereof or the hydrate thereof and the second dose of the first composition comprises about 600 mg of stenoparib or the pharmaceutically acceptable salt thereof or the hydrate thereof; and/or
(B)
(a) the dose of the second composition comprises about 300 mg of dovitinib or the pharmaceutically acceptable salt thereof or the hydrate thereof;
(b) the dose of the second composition comprises about 400 mg of dovitinib or the pharmaceutically acceptable salt thereof or the hydrate thereof; or
(c) the dose of the second composition comprises about 500 mg of dovitinib or the pharmaceutically acceptable salt thereof or the hydrate thereof.

37-38. (canceled)

39. The method claim 25, wherein the cancer:
(a) is a solid tumor cancer;
(b) is locally advanced or metastatic;
(c) comprises a BRCA mutation; and/or
(d) is homologous recombination (HR) deficient.

40. The method of claim 25, wherein the cancer is a solid tumor cancer, and wherein the cancer is an ovarian cancer, a breast cancer, a colon cancer, a pancreatic cancer, or a prostate cancer.

41-43. (canceled)

44. The method of claim 25, wherein:
(a) the subject is administered the first composition orally;
(b) the first composition is formulated as a capsule or a tablet;
(c) the subject is administered the second composition orally; and/or
(d) the second composition is formulated as a tablet.

45. (canceled)

46. The method of claim 44, wherein:
(a) the first composition is formulated as a capsule, and wherein each capsule of the first composition comprises about 50 mg, about 100 mg, or about 200 mg of stenoparib or the pharmaceutically acceptable salt thereof or the hydrate thereof; and/or
(b) the second composition is formulated as a tablet, and wherein each tablet of the second composition comprises about 100 mg of dovitinib or the pharmaceutically acceptable salt thereof or the hydrate thereof.

47-49. (canceled)

50. The method of claim 25, wherein the method comprises, prior to the administration of the first and second compositions:
(I) determining the responsiveness of the subject to treatment with stenoparib or a pharmaceutically acceptable salt thereof or a hydrate thereof by:
(a) determining the expression level of one or more biomarkers of sensitivity selected from the biomarkers of Table 1; and/or
(b) determining the expression level of one or more biomarkers of resistance selected from the biomarkers of Table 2; and/or
(II) determining the responsiveness of the subject to treatment with dovitinib or a pharmaceutically acceptable salt thereof or a hydrate thereof by:
(a) determining the expression level of one or more biomarkers of sensitivity selected from the biomarkers of Table 3; and/or
(b) determining the expression level of one or more biomarkers of resistance selected from the biomarkers of Table 4.

51-99. (canceled)


SEQUENCE LISTING (472 sequences)

SEQ ID NO: 1 - DNA, 25 bp, synthetic construct
  source 1..25: mol_type = other DNA
gatcaagatctatctctcttcgtag

SEQ ID NO: 2 - DNA, 25 bp, synthetic construct
  source 1..25: mol_type = other DNA
gagggactttgctgatttcccctct

SEQ ID NO: 3 - DNA, 25 bp, synthetic construct
  source 1..25: mol_type = other DNA
tcagtgggcacagttcttcagctac

SEQ ID NO: 4 - DNA, 25 bp, synthetic construct
  source 1..25: mol_type = other DNA
cctggagcaatgcactgtacctgcc

SEQ ID NO: 5 - DNA, 25 bp, synthetic construct
  source 1..25: mol_type = other DNA
gtctgtgtctgacaatcgtcacgca

SEQ ID NO: 6 - DNA, 25 bp, synthetic construct
  source 1..25: mol_type = other DNA
tccccgtacacgcactggaagcaga

SEQ ID NO: 7 - DNA, 25 bp, synthetic construct
  source 1..25: mol_type = other DNA
ttgaagatccccgagatgcagctga

SEQ ID NO: 8 - DNA, 25 bp, synthetic construct
  source 1..25: mol_type = other DNA
gcgtttctggcgatgcagcaattcc

SEQ ID NO: 9 - DNA, 25 bp, synthetic construct
  source 1..25: mol_type = other DNA
aaccatgcctactaaatgtacagat

SEQ ID NO: 10 - DNA, 25 bp, synthetic construct
  source 1..25: mol_type = other DNA
agatcattaaaatccgtgcccagac

SEQ ID NO: 11 - DNA, 25 bp, synthetic construct
  source 1..25: mol_type = other DNA
ttactcaagttcaaacctccagcct

SEQ ID NO: 12 - DNA, 25 bp, synthetic construct
  source 1..25: mol_type = other DNA
tgttctagatttcctgtagctgtga

SEQ ID NO: 13 - DNA, 25 bp, synthetic construct
  source 1..25: mol_type = other DNA
gtgctttagttgctagtttgtactg

SEQ ID NO: 14 - DNA, 25 bp, synthetic construct
  source 1..25: mol_type = other DNA
cctgcgagaagtcatctgcacgtcc

SEQ ID NO: 15 - DNA, 25 bp, synthetic construct
  source 1..25: mol_type = other DNA
gccattaacagtgttttctttccca

SEQ ID NO: 16 - DNA, 25 bp, synthetic construct
  source 1..25: mol_type = other DNA
cgcagccacgccgatggttatacag

SEQ ID NO: 17 - DNA, 25 bp, synthetic construct
  source 1..25: mol_type = other DNA
tcttgctaaaaccggcaattctccg

SEQ ID NO: 18 - DNA, 25 bp, synthetic construct
  source 1..25: mol_type = other DNA
tgactccatttctgtaagctactct

SEQ ID NO: 19 - DNA, 25 bp, synthetic construct
  source 1..25: mol_type = other DNA
ggtggtctttcaggttatcttggca

SEQ ID NO: 20 - DNA, 25 bp, synthetic construct
  source 1..25: mol_type = other DNA
tgtgtcttagatcctcattatttta

SEQ ID NO: 21 - DNA, 25 bp, synthetic construct
  source 1..25: mol_type = other DNA
ctgtactcttcgcatggactgggaa

SEQ ID NO: 22 - DNA, 25 bp, synthetic construct
  source 1..25: mol_type = other DNA
cggctctagccactgagcggctaaa

SEQ ID NO: 23 - DNA, 25 bp, synthetic construct
  source 1..25: mol_type = other DNA
aagattggtgctttttgcatgtttg

SEQ ID NO: 24 - DNA, 25 bp, synthetic construct
  source 1..25: mol_type = other DNA
gccgagagagcctgccggatgatga

SEQ ID NO: 25 - DNA, 25 bp, synthetic construct
  source 1..25: mol_type = other DNA
ggagcaatctccttggaaggattca

SEQ ID NO: 26 - DNA, 25 bp, synthetic construct
  source 1..25: mol_type = other DNA
gaaaagtcatccctatttccttgtt

SEQ ID NO: 27 - DNA, 25 bp, synthetic construct
  source 1..25: mol_type = other DNA
agtgttgatgggctctacttctgat

SEQ ID NO: 28 - DNA, 25 bp, synthetic construct
  source 1..25: mol_type = other DNA
atttacctctaaatttccatatcct

SEQ ID NO: 29 - DNA, 25 bp, synthetic construct
  source 1..25: mol_type = other DNA
ggccttgtctaacttttgtgaagaa

SEQ ID NO: 30 - DNA, 25 bp, synthetic construct
  source 1..25: mol_type = other DNA
ccacagaaatccagcatggcaactc

SEQ ID NO: 31 - DNA, 25 bp, synthetic construct
  source 1..25: mol_type = other DNA
aagatgaagctctctttgctcttcc

SEQ ID NO: 32 - DNA, 25 bp, synthetic construct
  source 1..25: mol_type = other DNA
gagggggaggtttactctgcggatc

SEQ ID NO: 33 - DNA, 25 bp, synthetic construct
  source 1..25: mol_type = other DNA
atcgcatcatgccaacttgtgactt

SEQ ID NO: 34 - DNA, 25 bp, synthetic construct
  source 1..25: mol_type = other DNA
ttttcctggccgtggtagttactca

SEQ ID NO: 35 - DNA, 25 bp, synthetic construct
  source 1..25: mol_type = other DNA
tcatcctcttagaagcacctgtgaa

SEQ ID NO: 36 - DNA, 25 bp, synthetic construct
  source 1..25: mol_type = other DNA
ggtagaggcgcttattgccctaacc

SEQ ID NO: 37 - DNA, 25 bp, synthetic construct
  source 1..25: mol_type = other DNA
tctttccttcagctcctgatgttaa

SEQ ID NO: 38 - DNA, 25 bp, synthetic construct
  source 1..25: mol_type = other DNA
catccaggaccgtgttctttacgta

SEQ ID NO: 39 - DNA, 25 bp, synthetic construct
  source 1..25: mol_type = other DNA
tattccggaatgagaccctggacgc

SEQ ID NO: 40 - DNA, 25 bp, synthetic construct
  source 1..25: mol_type = other DNA
gctgcagctctgacacaacataatg

SEQ ID NO: 41 - DNA, 25 bp, synthetic construct
  source 1..25: mol_type = other DNA
gaggatcttgttcaatcggaaaccc

SEQ ID NO: 42 - DNA, 25 bp, synthetic construct
  source 1..25: mol_type = other DNA
tgatgttctcaagcctcggaagtgg

SEQ ID NO: 43 - DNA, 25 bp, synthetic construct
  source 1..25: mol_type = other DNA
ttaactacgacatgcctgaggactc

SEQ ID NO: 44 - DNA, 25 bp, synthetic construct
  source 1..25: mol_type = other DNA
ccagaggccgacgatctactatgtg

SEQ ID NO: 45 - DNA, 25 bp, synthetic construct
  source 1..25: mol_type = other DNA
gctacatacaacctagtacacttga

SEQ ID NO: 46 - DNA, 25 bp, synthetic construct
  source 1..25: mol_type = other DNA
aatgcggctgtagaacatgctgaaa

SEQ ID NO: 47 - DNA, 25 bp, synthetic construct
  source 1..25: mol_type = other DNA
ccttctccttactatagtcgtggag

SEQ ID NO: 48 - DNA, 25 bp, synthetic construct
  source 1..25: mol_type = other DNA
cacaggtcccggggcatcaggagaa

SEQ ID NO: 49 - DNA, 25 bp, synthetic construct
  source 1..25: mol_type = other DNA
ccaaggggtgctgtatgctctaggc

SEQ ID NO: 50 - DNA, 25 bp, synthetic construct
  source 1..25: mol_type = other DNA
cagtttccacgagagcacagaaggg

SEQ ID NO: 51 - DNA, 25 bp, synthetic construct
  source 1..25: mol_type = other DNA
ggcgccaatggcgagatctgcgtca

SEQ ID NO: 52 - DNA, 25 bp, synthetic construct
  source 1..25: mol_type = other DNA
gagaaggagcggaggatggccaaca

SEQ ID NO: 53 - DNA, 25 bp, synthetic construct
  source 1..25: mol_type = other DNA
gggtcaatgcattttggggtgagct

SEQ ID NO: 54 - DNA, 25 bp, synthetic construct
  source 1..25: mol_type = other DNA
caaaccttttcgttttactgatcat

SEQ ID NO: 55 - DNA, 25 bp, synthetic construct
  source 1..25: mol_type = other DNA
ttggtcaaggccattacctgtttcc

SEQ ID NO: 56 - DNA, 25 bp, synthetic construct
  source 1..25: mol_type = other DNA
tataccctttctacagagttgcctc

SEQ ID NO: 57 - DNA, 25 bp, synthetic construct
  source 1..25: mol_type = other DNA
gctgaacaggcctatcttgaacttt

SEQ ID NO: 58 - DNA, 25 bp, synthetic construct
  source 1..25: mol_type = other DNA
taatggccaaggctgtttgcatccc

SEQ ID NO: 59 - DNA, 25 bp, synthetic construct
  source 1..25: mol_type = other DNA
agcgaaagtgcattcagtccatcct

SEQ ID NO: 60 - DNA, 25 bp, synthetic construct
  source 1..25: mol_type = other DNA
actgattatggctcaggctacccca

SEQ ID NO: 61 - DNA, 25 bp, synthetic construct
  source 1..25: mol_type = other DNA
gtgatcgaggatgctcgacacccac

SEQ ID NO: 62 - DNA, 25 bp, synthetic construct
  source 1..25: mol_type = other DNA
ggcaggcaattctaagatcttccac

SEQ ID NO: 63 - DNA, 25 bp, synthetic construct
  source 1..25: mol_type = other DNA
gtgatcgtagtcatgcctcagaagc

SEQ ID NO: 64 - DNA, 25 bp, synthetic construct
  source 1..25: mol_type = other DNA
gatgggaagcgaaaggccaaggacc

SEQ ID NO: 65 - DNA, 25 bp, synthetic construct
  source 1..25: mol_type = other DNA
acagacatcaccagttgacttctgc

SEQ ID NO: 66 - DNA, 25 bp, synthetic construct
  source 1..25: mol_type = other DNA
gtaactaggtttttgcttctccagt

SEQ ID NO: 67 - DNA, 25 bp, synthetic construct
  source 1..25: mol_type = other DNA
cacagcttcctagccacagtgataa

SEQ ID NO: 68 - DNA, 25 bp, synthetic construct
  source 1..25: mol_type = other DNA
cggcagcgcaagatgagggtgaatg

SEQ ID NO: 69 - DNA, 25 bp, synthetic construct
  source 1..25: mol_type = other DNA
agatactcagcactagactaacata

SEQ ID NO: 70 - DNA, 25 bp, synthetic construct
  source 1..25: mol_type = other DNA
cattgctatatgtgtctgttctact

SEQ ID NO: 71 - DNA, 25 bp, synthetic construct
  source 1..25: mol_type = other DNA
gagacagctgcattgttgtggaatt

SEQ ID NO: 72 - DNA, 25 bp, synthetic construct
  source 1..25: mol_type = other DNA
gcctgatgggcctggtagttttcca

SEQ ID NO: 73 - DNA, 25 bp, synthetic construct
  source 1..25: mol_type = other DNA
gtaaccgtagcaaaactgcattggt

SEQ ID NO: 74 - DNA, 25 bp, synthetic construct
  source 1..25: mol_type = other DNA
agatcggcatagtggagccagtgct

SEQ ID NO: 75 - DNA, 25 bp, synthetic construct
  source 1..25: mol_type = other DNA
tttgtaaaatgatgcttcccccttc

SEQ ID NO: 76 - DNA, 25 bp, synthetic construct
  source 1..25: mol_type = other DNA
aaggagatcggcttgtggtttcacc

SEQ ID NO: 77 - DNA, 25 bp, synthetic construct
  source 1..25: mol_type = other DNA
acagcattgtacccagagtctgtcc

SEQ ID NO: 78 - DNA, 25 bp, synthetic construct
  source 1..25: mol_type = other DNA
tgcccaatcggaagagctctgtgcc

SEQ ID NO: 79 - DNA, 25 bp, synthetic construct
  source 1..25: mol_type = other DNA
aggtgccattattctttctagcctc

SEQ ID NO: 80 - DNA, 25 bp, synthetic construct
  source 1..25: mol_type = other DNA
gaagctgtccacgatgtcttggatc

SEQ ID NO: 81 - DNA, 25 bp, synthetic construct
  source 1..25: mol_type = other DNA
ggagcataaaagtgtaagaccgtaa

SEQ ID NO: 82 - DNA, 25 bp, synthetic construct
  source 1..25: mol_type = other DNA
gatctttcagattttccatctctag

SEQ ID NO: 83 - DNA, 25 bp, synthetic construct
  source 1..25: mol_type = other DNA
atgaccactgttagcccattatatt

SEQ ID NO: 84 - DNA, 25 bp, synthetic construct
  source 1..25: mol_type = other DNA
tagtatccagtccattgtacctgct

SEQ ID NO: 85 - DNA, 25 bp, synthetic construct
  source 1..25: mol_type = other DNA
agatggtctcatcggagtcgcattt

SEQ ID NO: 86 - DNA, 25 bp, synthetic construct
  source 1..25: mol_type = other DNA
atatctatgaccactgttagcccat

SEQ ID NO: 87 - DNA, 25 bp, synthetic construct
  source 1..25: mol_type = other DNA
taaagcctattctaccagttaaact

SEQ ID NO: 88 - DNA, 25 bp, synthetic construct
  source 1..25: mol_type = other DNA
ggagcatcagggttggcttgggagc

SEQ ID NO: 89 - DNA, 25 bp, synthetic construct
  source 1..25: mol_type = other DNA
cattgacctcgagggcaacgtctgc

SEQ ID NO: 90 - DNA, 25 bp, synthetic construct
  source 1..25: mol_type = other DNA
ttatacgtttatatgctctgtctgc

SEQ ID NO: 91 - DNA, 25 bp, synthetic construct
  source 1..25: mol_type = other DNA
gttggccacaacattatgattattc

SEQ ID NO: 92 - DNA, 25 bp, synthetic construct
  source 1..25: mol_type = other DNA
tcctgtgtggtgtgtctactgtgag

SEQ ID NO: 93 - DNA, 25 bp, synthetic construct
  source 1..25: mol_type = other DNA
agagctggggttaggatccggttgg

SEQ ID NO: 94 - DNA, 25 bp, synthetic construct
  source 1..25: mol_type = other DNA
tcctttctgttttgcgagcttgtgt

SEQ ID NO: 95 - DNA, 25 bp, synthetic construct
  source 1..25: mol_type = other DNA
aaaagcccagtgtgacagtgtcatg

SEQ ID NO: 96 - DNA, 25 bp, synthetic construct
  source 1..25: mol_type = other DNA
cagtacaagcgctgcggtgtcacct

SEQ ID NO: 97 - DNA, 25 bp, synthetic construct
  source 1..25: mol_type = other DNA
gaaagccaacagacggtgcttcctc

SEQ ID NO: 98 - DNA, 25 bp, synthetic construct
  source 1..25: mol_type = other DNA
gtttacctctggatacactacttgt

SEQ ID NO: 99 - DNA, 25 bp, synthetic construct
  source 1..25: mol_type = other DNA
cgctctgccgagaccaggacgagct

SEQ ID NO: 100 - DNA, 25 bp, synthetic construct
  source 1..25: mol_type = other DNA
caccgacgtcagcaacttcgacgag

SEQ ID NO: 101 - DNA, 25 bp, synthetic construct
  source 1..25: mol_type = other DNA
aagcctactagagccattgtatgtg

SEQ ID NO: 102 - DNA, 25 bp, synthetic construct
  source 1..25: mol_type = other DNA
cttcattactgaacaaaactacggt

SEQ ID NO: 103 - DNA, 25 bp, synthetic construct
  source 1..25: mol_type = other DNA
tgctcatcaactgccgcaacaataa

SEQ ID NO: 104 - DNA, 25 bp, synthetic construct
  source 1..25: mol_type = other DNA
atcccacctgtgagaatatgaactt

SEQ ID NO: 105 - DNA, 25 bp, synthetic construct
  source 1..25: mol_type = other DNA
agctaaacaccaatttcttcctgct

SEQ ID NO: 106 - DNA, 25 bp, synthetic construct
  source 1..25: mol_type = other DNA
tagtgagctgcctgatgagatagac

SEQ ID NO: 107 - DNA, 25 bp, synthetic construct
  source 1..25: mol_type = other DNA
gcagctatcttaccagactgtgcct

SEQ ID NO: 108 - DNA, 25 bp, synthetic construct
  source 1..25: mol_type = other DNA
gaagttcatggatcaacatccggag

SEQ ID NO: 109 - DNA, 25 bp, synthetic construct
  source 1..25: mol_type = other DNA
ggtgacataaaccattcattgctac

SEQ ID NO: 110 - DNA, 25 bp, synthetic construct
  source 1..25: mol_type = other DNA
aagtgcccaaataagtctgagtgct

SEQ ID NO: 111 - DNA, 25 bp, synthetic construct
  source 1..25: mol_type = other DNA
tttgacaaggagactggctttcaca

SEQ ID NO: 112 - DNA, 25 bp, synthetic construct
  source 1..25: mol_type = other DNA
aagtttgaatagcttctgtccctct

SEQ ID NO: 113 - DNA, 25 bp, synthetic construct
  source 1..25: mol_type = other DNA
tttggagttctcttacgtttcctgg

SEQ ID NO: 114 - DNA, 25 bp, synthetic construct
  source 1..25: mol_type = other DNA
tggcgagagaagcatgtccggtcac

SEQ ID NO: 115 - DNA, 25 bp, synthetic construct
  source 1..25: mol_type = other DNA
aataagtgccgggagctttgtgaga

SEQ ID NO: 116 - DNA, 25 bp, synthetic construct
  source 1..25: mol_type = other DNA
ggccagaaaccttgggtcttttcat

SEQ ID NO: 117 - DNA, 25 bp, synthetic construct
  source 1..25: mol_type = other DNA
ggatgtgggtctgattgcaattcgg

SEQ ID NO: 118 - DNA, 25 bp, synthetic construct
  source 1..25: mol_type = other DNA
tggaccatggtctagcagtagcatc

SEQ ID NO: 119 - DNA, 25 bp, synthetic construct
  source 1..25: mol_type = other DNA
gggcattcgcgtggtgaaggacctc

SEQ ID NO: 120 - DNA, 25 bp, synthetic construct
  source 1..25: mol_type = other DNA
gctgtaccccaaggaagacaaggag

SEQ ID NO: 121 - DNA, 25 bp, synthetic construct
  source 1..25: mol_type = other DNA
aaatctctgaactgtgtgggttttg

SEQ ID NO: 122 - DNA, 25 bp, synthetic construct
  source 1..25: mol_type = other DNA
tgccctcaaacatacagaacttcca

SEQ ID NO: 123 - DNA, 25 bp, synthetic construct
  source 1..25: mol_type = other DNA
tcatctctagcagtatggcatcccc

SEQ ID NO: 124 - DNA, 25 bp, synthetic construct
  source 1..25: mol_type = other DNA
gcaaggattgcggcgtgggtttccg

SEQ ID NO: 125 - DNA, 25 bp, synthetic construct
  source 1..25: mol_type = other DNA
tcaatggccgctggtttgctggccg

SEQ ID NO: 126 - DNA, 25 bp, synthetic construct
  source 1..25: mol_type = other DNA
cacaccagctctactctttagtaaa

SEQ ID NO: 127 - DNA, 25 bp, synthetic construct
  source 1..25: mol_type = other DNA
agaaactcaccctaaatctgaacgg

SEQ ID NO: 128 - DNA, 25 bp, synthetic construct
  source 1..25: mol_type = other DNA
aagtggtaacaggactgatgccgaa

SEQ ID NO: 129 - DNA, 25 bp, synthetic construct
  source 1..25: mol_type = other DNA
tgtttatagatcactgccctttttg

SEQ ID NO: 130 - DNA, 25 bp, synthetic construct
  source 1..25: mol_type = other DNA
aagtcactcaggtcatgggcattgt

SEQ ID NO: 131 - DNA, 25 bp, synthetic construct
  source 1..25: mol_type = other DNA
tgattactacctcaggaccaacctc

SEQ ID NO: 132 - DNA, 25 bp, synthetic construct
  source 1..25: mol_type = other DNA
gtacttggataggctggctaactcg

SEQ ID NO: 133 - DNA, 25 bp, synthetic construct
  source 1..25: mol_type = other DNA
gaagctcgagagcccttaagttctg

SEQ ID NO: 134 - DNA, 25 bp, synthetic construct
  source 1..25: mol_type = other DNA
ccatcgacaacacaaacccagacgc

SEQ ID NO: 135 - DNA, 25 bp, synthetic construct
  source 1..25: mol_type = other DNA
agcttgttgttgatcagattcactg

SEQ ID NO: 136 - DNA, 25 bp, synthetic construct
  source 1..25: mol_type = other DNA
gcagagcattcagtgccacggttta

SEQ ID NO: 137 - DNA, 25 bp, synthetic construct
  source 1..25: mol_type = other DNA
ctcttcagcagcatctactctaggc

SEQ ID NO: 138 - DNA, 25 bp, synthetic construct
  source 1..25: mol_type = other DNA
ctgctgtaaggactggttccagaat

SEQ ID NO: 139 - DNA, 25 bp, synthetic construct
  source 1..25: mol_type = other DNA
gaacttgccacatacccaggtataa

SEQ ID NO: 140 - DNA, 25 bp, synthetic construct
  source 1..25: mol_type = other DNA
catgaaggcacccttgaaccatcca

SEQ ID NO: 141 - DNA, 25 bp, synthetic construct
  source 1..25: mol_type = other DNA
ggagccattggctgtgaagctgcag

SEQ ID NO: 142 - DNA, 25 bp, synthetic construct
  source 1..25: mol_type = other DNA
aatctgaccctatcggaaactcaaa

SEQ ID NO: 143 - DNA, 25 bp, synthetic construct
  source 1..25: mol_type = other DNA
atagcctcaggaacagtccagactg

SEQ ID NO: 144 - DNA, 25 bp, synthetic construct
  source 1..25: mol_type = other DNA
ggtggtgatcaaaactatagtggct

SEQ ID NO: 145 - DNA, 25 bp, synthetic construct
  source 1..25: mol_type = other DNA
tgatcaccctgcaatcctattatgt

SEQ ID NO: 146 - DNA, 25 bp, synthetic construct
  source 1..25: mol_type = other DNA
acttagaatccatctcactaccaat

SEQ ID NO: 147 - DNA, 25 bp, synthetic construct
  source 1..25: mol_type = other DNA
tagagactatgtcgctttcctgagc

SEQ ID NO: 148 - DNA, 25 bp, synthetic construct
  source 1..25: mol_type = other DNA
tactgtgatgggcagaacagccttt

SEQ ID NO: 149 - DNA, 25 bp, synthetic construct
  source 1..25: mol_type = other DNA
tctcatttaggactgggttccccac

SEQ ID NO: 150 - DNA, 25 bp, synthetic construct
  source 1..25: mol_type = other DNA
acagcttttaacacagttccctgcc

SEQ ID NO: 151 - DNA, 25 bp, synthetic construct
  source 1..25: mol_type = other DNA
gatgaaagatgagcccaagtccacc

SEQ ID NO: 152 - DNA, 25 bp, synthetic construct
  source 1..25: mol_type = other DNA
gatcctcttaaaacgtttatattct

SEQ ID NO: 153 - DNA, 25 bp, synthetic construct
  source 1..25: mol_type = other DNA
ccctggtcacatgtcatcgggctgg

SEQ ID NO: 154 - DNA, 25 bp, synthetic construct
  source 1..25: mol_type = other DNA
atccacatcagaatcccttgtagga

SEQ ID NO: 155 - DNA, 25 bp, synthetic construct
  source 1..25: mol_type = other DNA
actgctttcaagctggactgagcca

SEQ ID NO: 156 - DNA, 25 bp, synthetic construct
  source 1..25: mol_type = other DNA
taaacattccgtccctgtttgagac

SEQ ID NO: 157 - DNA, 25 bp, synthetic construct
  source 1..25: mol_type = other DNA
gtgagatcaaagctcctccaaagcc

SEQ ID NO: 158 - DNA, 25 bp, synthetic construct
  source 1..25: mol_type = other DNA
cctcggtggccctgactgtcaggga

SEQ ID NO: 159 - DNA, 25 bp, synthetic construct
  source 1..25: mol_type = other DNA
tcacgccgatgatggagctgaagcc

SEQ ID NO: 160 - DNA, 25 bp, synthetic construct
  source 1..25: mol_type = other DNA
gagttctatttgagacttccagccc

SEQ ID NO: 161 - DNA, 25 bp, synthetic construct
  source 1..25: mol_type = other DNA
ccaaaggaggcattatgcttccaga

SEQ ID NO: 162 - DNA, 25 bp, synthetic construct
  source 1..25: mol_type = other DNA
gaagtgtgctagtatgctccctagt

SEQ ID NO: 163 - DNA, 25 bp, synthetic construct
  source 1..25: mol_type = other DNA
gttggggttggagacgtgtgtaata

SEQ ID NO: 164 - DNA, 25 bp, synthetic construct
  source 1..25: mol_type = other DNA
ggaggctaagtattgctttctacaa

SEQ ID NO: 165 - DNA, 25 bp, synthetic construct
  source 1..25: mol_type = other DNA
tcattagctctgccctttttaatta

SEQ ID NO: 166 - DNA, 25 bp, synthetic construct
  source 1..25: mol_type = other DNA
cacttctcctttacgctgtgtagag

SEQ ID NO: 167 - DNA, 25 bp, synthetic construct
  source 1..25: mol_type = other DNA
gtactttgatactgctcatgctgac

SEQ ID NO: 168 - DNA, 25 bp, synthetic construct
  source 1..25: mol_type = other DNA
caggggatggcctgatcgagctgcg

SEQ ID NO: 169 - DNA, 25 bp, synthetic construct
  source 1..25: mol_type = other DNA
ccttattccattcttaactctgcat

SEQ ID NO: 170 - DNA, 25 bp, synthetic construct
  source 1..25: mol_type = other DNA
gcagtcactctccctgaataagtaa

SEQ ID NO: 171 - DNA, 25 bp, synthetic construct
  source 1..25: mol_type = other DNA
gaagaagaactagccagtactcccc

SEQ ID NO: 172 - DNA, 25 bp, synthetic construct
  source 1..25: mol_type = other DNA
ggagtgccaagtttgcaaacctgca

SEQ ID NO: 173 - DNA, 25 bp, synthetic construct
  source 1..25: mol_type = other DNA
aatgggttatcacaggaatgggact

SEQ ID NO: 174 - DNA, 25 bp, synthetic construct
  source 1..25: mol_type = other DNA
tgggcagagtgcggcagctcatgcc

SEQ ID NO: 175 - DNA, 25 bp, synthetic construct
  source 1..25: mol_type = other DNA
tggagtgagactgactgcaagcccc

SEQ ID NO: 176 - DNA, 25 bp, synthetic construct
  source 1..25: mol_type = other DNA
tttttggatggcaacgagctcaccc

SEQ ID NO: 177 - DNA, 25 bp, synthetic construct
  source 1..25: mol_type = other DNA
ttattgcttaatttctgcctttccc

SEQ ID NO: 178 - DNA, 25 bp, synthetic construct
  source 1..25: mol_type = other DNA
gctgccttgtgtgcgactgagatgc

SEQ ID NO: 179 - DNA, 25 bp, synthetic construct
  source 1..25: mol_type = other DNA
ctacgatgagtttgagtactgctgg

SEQ ID NO: 180 - DNA, 25 bp, synthetic construct
  source 1..25: mol_type = other DNA
ctcctggactcaatcatggcttgtg

SEQ ID NO: 181 - DNA, 25 bp, synthetic construct
  source 1..25: mol_type = other DNA
tggagggcgctcatcaagtagctgc

SEQ ID NO: 182 - DNA, 25 bp, synthetic construct
  source 1..25: mol_type = other DNA
tggtcccagccagtttggtgctgac

SEQ ID NO: 183 - DNA, 25 bp, synthetic construct
  source 1..25: mol_type = other DNA
gatcctttctgtaggctaattcctc

SEQ ID NO: 184 - DNA, 25 bp, synthetic construct
  source 1..25: mol_type = other DNA
ttcccagaagcggacctgaggaccc

SEQ ID NO: 185 - DNA, 25 bp, synthetic construct
  source 1..25: mol_type = other DNA
gaccttcaagggtcctcgttttgat

SEQ ID NO: 186 - DNA, 25 bp, synthetic construct
  source 1..25: mol_type = other DNA
gacggtgcttttcggtgcaaggaaa

SEQ ID NO: 187 - DNA, 25 bp, synthetic construct
  source 1..25: mol_type = other DNA
tgaacttgttgagatccccgtgtta

SEQ ID NO: 188 - DNA, 25 bp, synthetic construct
  source 1..25: mol_type = other DNA
ttctactcaggcacgggctacaagc

SEQ ID NO: 189 - DNA, 25 bp, synthetic construct
  source 1..25: mol_type = other DNA
ccggtgtccttgaccacatgaagaa

SEQ ID NO: 190 - DNA, 25 bp, synthetic construct
  source 1..25: mol_type = other DNA
tgatgcactgattccatcccaggaa

SEQ ID NO: 191 - DNA, 25 bp, synthetic construct
  source 1..25: mol_type = other DNA
tgacagtcactagcttatcttgaac

SEQ ID NO: 192 - DNA, 25 bp, synthetic construct
  source 1..25: mol_type = other DNA
ttgtgtgaagtggttcacccttgag

SEQ ID NO: 193 - DNA, 25 bp, synthetic construct
  source 1..25: mol_type = other DNA
gacttccggatcctgtcagggtgtc

SEQ ID NO: 194 - DNA, 25 bp, synthetic construct
  source 1..25: mol_type = other DNA
gacaaagatcttgccttacagactt

SEQ ID NO: 195 - DNA, 25 bp, synthetic construct
  source 1..25: mol_type = other DNA
tggtcgcagctgtgctgttcatcac

SEQ ID NO: 196 - DNA, 25 bp, synthetic construct
  source 1..25: mol_type = other DNA
gtgacttcaagagcctctggcatct

SEQ ID NO: 197 - DNA, 25 bp, synthetic construct
  source 1..25: mol_type = other DNA
gagaacaacagcattcatttccatt

SEQ ID NO: 198 - DNA, 25 bp, synthetic construct
  source 1..25: mol_type = other DNA
ccctcaccctgagatgggagccgtc

SEQ ID NO: 199 - DNA, 25 bp, synthetic construct
  source 1..25: mol_type = other DNA
caccatccccaacttgggcatcgtt

SEQ ID NO: 200 - DNA, 25 bp, synthetic construct
  source 1..25: mol_type = other DNA
agtaaatgattcctccttgttctgt

SEQ ID NO: 201 - DNA, 25 bp, synthetic construct
  source 1..25: mol_type = other DNA
caaaacgggcatcacccagttgacc

SEQ ID NO: 202 - DNA, 25 bp, synthetic construct
  source 1..25: mol_type = other DNA
aaggagggcacctctgtctgagttt

SEQ ID NO: 203 - DNA, 25 bp, synthetic construct
  source 1..25: mol_type = other DNA
ttatttatacagtgccttgctcggg

SEQ ID NO: 204 - DNA, 25 bp, synthetic construct
  source 1..25: mol_type = other DNA
ttcggggacaatctggtcacccgct

SEQ ID NO: 205 - DNA, 25 bp, synthetic construct
  source 1..25: mol_type = other DNA
cctcattgagttcggtgcatctggc

SEQ ID NO: 206 - DNA, 25 bp, synthetic construct
  source 1..25: mol_type = other DNA
tgcttgctaccgtgttcggagtctc

SEQ ID NO: 207 - DNA, 25 bp, synthetic construct
  source 1..25: mol_type = other DNA
ccctgcacatgaagaggcacctctg

SEQ ID NO: 208 - DNA, 25 bp, synthetic construct
  source 1..25: mol_type = other DNA
ggctgtcctagcagttgtggtcatc

SEQ ID NO: 209 - DNA, 25 bp, synthetic construct
  source 1..25: mol_type = other DNA
gtcacacaccagcactttatacact

SEQ ID NO: 210 - DNA, 25 bp, synthetic construct
  source 1..25: mol_type = other DNA
gaacactctactacatgtgactggc

SEQ ID NO: 211 - DNA, 25 bp, synthetic construct
  source 1..25: mol_type = other DNA
gaggactactctggatcaggcttcg

SEQ ID NO: 212 - DNA, 25 bp, synthetic construct
  source 1..25: mol_type = other DNA
gagctagtggatgtgtttgtctaca

SEQ ID NO: 213 - DNA, 25 bp, synthetic construct
  source 1..25: mol_type = other DNA
ttccttccttatcactgacacaaaa

SEQ ID NO: 214 - DNA, 25 bp, synthetic construct
  source 1..25: mol_type = other DNA
ggactctattccatctatcaagggg

SEQ ID NO: 215 - DNA, 25 bp, synthetic construct
  source 1..25: mol_type = other DNA
ccccatcgtgggcattgttgctggc

SEQ ID NO: 216 - DNA, 25 bp, synthetic construct
  source 1..25: mol_type = other DNA
gcggattcttcagcgagatctcgca

SEQ ID NO: 217 - DNA, 25 bp, synthetic construct
  source 1..25: mol_type = other DNA
ctcaccagtgcttcatataccatga

SEQ ID NO: 218 - DNA, 25 bp, synthetic construct
  source 1..25: mol_type = other DNA
gcaagtttcatcagccctagggaaa

SEQ ID NO: 219 - DNA, 25 bp, synthetic construct
  source 1..25: mol_type = other DNA
ctcagcttccctcttaagctaagac

SEQ ID NO: 220 - DNA, 25 bp, synthetic construct
  source 1..25: mol_type = other DNA
ccccatcgtgggcattgttgctggc

SEQ ID NO: 221 - DNA, 25 bp, synthetic construct
  source 1..25: mol_type = other DNA
gaatgttctccaagcagcaatccaa

SEQ ID NO: 222 - DNA, 25 bp, synthetic construct
  source 1..25: mol_type = other DNA
atgtgcgtgtcgtcgctgtttagac

SEQ ID NO: 223 - DNA, 25 bp, synthetic construct
  source 1..25: mol_type = other DNA
cacaagcccctgaaccatatattgt

SEQ ID NO: 224 - DNA, 25 bp, synthetic construct
  source 1..25: mol_type = other DNA
gatgtggacgttcgggagctactgc

SEQ ID NO: 225 - DNA, 25 bp, synthetic construct
  source 1..25: mol_type = other DNA
tcctgttgtttgctgccattggcat

SEQ ID NO: 226 - DNA, 25 bp, synthetic construct
  source 1..25: mol_type = other DNA
gtggccagcactctgaaactgagaa

SEQ ID NO: 227 - DNA, 25 bp, synthetic construct
  source 1..25: mol_type = other DNA
agttccgcttgtccaagatctggtc

SEQ ID NO: 228 - DNA, 25 bp, synthetic construct
  source 1..25: mol_type = other DNA
agagccccagtttgtaaatgaacct

SEQ ID NO: 229 - DNA, 25 bp, synthetic construct
  source 1..25: mol_type = other DNA
gtccccaagtgtgcaagctcagtat

SEQ ID NO: 230 - DNA, 25 bp, synthetic construct
  source 1..25: mol_type = other DNA
gacgtggagctcgtggagaccaggc

SEQ ID NO: 231 - DNA, 25 bp, synthetic construct
  source 1..25: mol_type = other DNA
tttttgcttcaagctctgtagcagg

SEQ ID NO: 232 - DNA, 25 bp, synthetic construct
  source 1..25: mol_type = other DNA
tccatttctggcagcagcctggagc

SEQ ID NO: 233 - DNA, 25 bp, synthetic construct
  source 1..25: mol_type = other DNA
tctaccccaggtcagacggacagaa

SEQ ID NO: 234 - DNA, 25 bp, synthetic construct
  source 1..25: mol_type = other DNA
acgggctgtccgtgtgcctcctgtg

SEQ ID NO: 235 - DNA, 25 bp, synthetic construct
  source 1..25: mol_type = other DNA
ggtggagtcagtgaccaggtcgtct

SEQ ID NO: 236 - DNA, 25 bp, synthetic construct
  source 1..25: mol_type = other DNA
ctaacccaccggcagagtatttttg

SEQ ID NO: 237 - DNA, 25 bp, synthetic construct
  source 1..25: mol_type = other DNA
gctttcgaagtggccagctgcggcc

SEQ ID NO: 238 - DNA, 25 bp, synthetic construct
  source 1..25: mol_type = other DNA
gtgttagaagcagctgtgggggtcc

SEQ ID NO: 239 - DNA, 25 bp, synthetic construct
  source 1..25: mol_type = other DNA
gagtgaggcgatttgacctgctcaa

SEQ ID NO: 240 - DNA, 25 bp, synthetic construct
  source 1..25: mol_type = other DNA
taagggcttgattcctagccccgtt

SEQ ID NO: 241 - DNA, 25 bp, synthetic construct
  source 1..25: mol_type = other DNA
aggggcctctgaacggaacagtgtc

SEQ ID NO: 242 - DNA, 25 bp, synthetic construct
  source 1..25: mol_type = other DNA
tgagcacgctcagttctactgtgtc

SEQ ID NO: 243 - DNA, 25 bp, synthetic construct
  source 1..25: mol_type = other DNA
gtcttcgtccgataccaataagtca

SEQ ID NO: 244 - DNA, 25 bp, synthetic construct
  source 1..25: mol_type = other DNA
agctgagccacatcaagacacggtc

SEQ ID NO: 245 - DNA, 25 bp, synthetic construct
  source 1..25: mol_type = other DNA
cttatttaccggaagcttctgttca

SEQ ID NO: 246 - DNA, 25 bp, synthetic construct
  source 1..25: mol_type = other DNA
tgccttcatcctaactgctggggaa

SEQ ID NO: 247 - DNA, 25 bp, synthetic construct
  source 1..25: mol_type = other DNA
gaggcaccactaaaagatcgcagtt

SEQ ID NO: 248 - DNA, 25 bp, synthetic construct
  source 1..25: mol_type = other DNA
taccctgcggagatcatactgacct

SEQ ID NO: 249 - DNA, 25 bp, synthetic construct
  source 1..25: mol_type = other DNA
ggatgggaagtgttgcattgagcat

SEQ ID NO: 250 - DNA, 25 bp, synthetic construct
  source 1..25: mol_type = other DNA
cttggagatatttccgtcctccacc

SEQ ID NO: 251 - DNA, 25 bp, synthetic construct
  source 1..25: mol_type = other DNA
aggcctctcatgacccaggaaggcc

SEQ ID NO: 252 - DNA, 25 bp, synthetic construct
  source 1..25: mol_type = other DNA
tcatcactattattgctcaccactg

SEQ ID NO: 253 - DNA, 25 bp, synthetic construct
  source 1..25: mol_type = other DNA
aagctgacacctaagtttaccaaca

SEQ ID NO: 254 - DNA, 25 bp, synthetic construct
  source 1..25: mol_type = other DNA
taggtgtctgccttcacaaatgtca

SEQ ID NO: 255 - DNA, 25 bp, synthetic construct
  source 1..25: mol_type = other DNA
atattccctatgtagcaacagtggt

SEQ ID NO: 256 - DNA, 25 bp, synthetic construct
  source 1..25: mol_type = other DNA
gatcatgctgaaaaccactcaaacg

SEQ ID NO: 257 - DNA, 25 bp, synthetic construct
  source 1..25: mol_type = other DNA
aaggtccataaagactgagcggatg

SEQ ID NO: 258 - DNA, 25 bp, synthetic construct
  source 1..25: mol_type = other DNA
ctgaggagtccaacatcaccatgca

SEQ ID NO: 259 - DNA, 25 bp, synthetic construct
  source 1..25: mol_type = other DNA
ggacgacacctctggtttttcaata

SEQ ID NO: 260 - DNA, 25 bp, synthetic construct
  source 1..25: mol_type = other DNA
ttcactccatccctatggcagagga

SEQ ID NO: 261 - DNA, 25 bp, synthetic construct
  source 1..25: mol_type = other DNA
gagatgcatttaaggccgatagtga

SEQ ID NO: 262 - DNA, 25 bp, synthetic construct
  source 1..25: mol_type = other DNA
tcgttacagggagtccaacaccgcc

SEQ ID NO: 263 - DNA, 25 bp, synthetic construct
  source 1..25: mol_type = other DNA
gatggtccgaatggtttccaggtgg

SEQ ID NO: 264 - DNA, 25 bp, synthetic construct
  source 1..25: mol_type = other DNA
gtttttgtgctgttactcggtagag

SEQ ID NO: 265 - DNA, 25 bp, synthetic construct
  source 1..25: mol_type = other DNA
gagatgtgagccttgtgcatgtggg

SEQ ID NO: 266 - DNA, 25 bp, synthetic construct
  source 1..25: mol_type = other DNA
tgagatccgtgagatccttccatct

SEQ ID NO: 267 - DNA, 25 bp, synthetic construct
  source 1..25: mol_type = other DNA
tattattttctatgtccgttgttgt

SEQ ID NO: 268 - DNA, 25 bp, synthetic construct
  source 1..25: mol_type = other DNA
tactgggcaagtgcttgtcaagttc

SEQ ID NO: 269 - DNA, 25 bp, synthetic construct
  source 1..25: mol_type = other DNA
gaggatgccccaacaaactcatggc

SEQ ID NO: 270 - DNA, 25 bp, synthetic construct
  source 1..25: mol_type = other DNA
tctgagttgttggggctaagcctga

SEQ ID NO: 271 - DNA, 25 bp, synthetic construct
  source 1..25: mol_type = other DNA
gaaaggtaacaatcttcattctaca

SEQ ID NO: 272 - DNA, 25 bp, synthetic construct
  source 1..25: mol_type = other DNA
atcgatacagaaaccacgctgccgc

SEQ ID NO: 273 - DNA, 25 bp, synthetic construct
  source 1..25: mol_type = other DNA
actcttgagggttgattatgctgca

SEQ ID NO: 274 - DNA, 25 bp, synthetic construct
  source 1..25: mol_type = other DNA
ccctgagcaccgtgttcatgaatct

SEQ ID NO: 275 - DNA, 25 bp, synthetic construct
  source 1..25: mol_type = other DNA
gattgagtcatcgacattcaggatt

SEQ ID NO: 276 - DNA, 25 bp, synthetic construct
  source 1..25: mol_type = other DNA
cacccgggactacttgctggtgact

SEQ ID NO: 277 - DNA, 25 bp, synthetic construct
  source 1..25: mol_type = other DNA
tggagaaaatgacgacccacgacca

SEQ ID NO: 278 - DNA, 25 bp, synthetic construct
  source 1..25: mol_type = other DNA
gacagccttttgccattggtggctc

SEQ ID NO: 279 - DNA, 25 bp, synthetic construct
  source 1..25: mol_type = other DNA
ggcctttcacaactaggactgagaa

SEQ ID NO: 280 - DNA, 25 bp, synthetic construct
  source 1..25: mol_type = other DNA
tatgtaatgtacacctgccctaagg

SEQ ID NO: 281 - DNA, 25 bp, synthetic construct
  source 1..25: mol_type = other DNA
gggagagttgaccgttttcatcctg

SEQ ID NO: 282 - DNA, 25 bp, synthetic construct
  source 1..25: mol_type = other DNA
cataaggcactgggactcggatttc

SEQ ID NO: 283 - DNA, 25 bp, synthetic construct
  source 1..25: mol_type = other DNA
gcacatctgggcagttgtaccatgc

SEQ ID NO: 284 - DNA, 25 bp, synthetic construct
  source 1..25: mol_type = other DNA
cactgacagtgcccagggctctggg

SEQ ID NO: 285 - DNA, 25 bp, synthetic construct
  source 1..25: mol_type = other DNA
gagtgggagccgtgaatatctctgt

SEQ ID NO: 286 - DNA, 25 bp, synthetic construct
  source 1..25: mol_type = other DNA
aaattatcatactaccggctacatc

SEQ ID NO: 287 - DNA, 25 bp, synthetic construct
  source 1..25: mol_type = other DNA
taatttgcttcatttccttgctatt

SEQ ID NO: 288 - DNA, 25 bp, synthetic construct
  source 1..25: mol_type = other DNA
aaggtgaatgtaccctaacctgctc

SEQ ID NO: 289 - DNA, 25 bp, synthetic construct
  source 1..25: mol_type = other DNA
gagcatcaatcttgttactgctgat

SEQ ID NO: 290 - DNA, 25 bp, synthetic construct
  source 1..25: mol_type = other DNA
gatccccaagtggtgaatgacacgc

SEQ ID NO: 291 - DNA, 25 bp, synthetic construct
  source 1..25: mol_type = other DNA
gttacctactttgactttttgcatt

SEQ ID NO: 292 - DNA, 25 bp, synthetic construct
  source 1..25: mol_type = other DNA
gaacgggaaggagacgctgcagcgc

SEQ ID NO: 293 - DNA, 25 bp, synthetic construct
  source 1..25: mol_type = other DNA
cagactggaaacgggactttctatt

SEQ ID NO: 294 - DNA, 25 bp, synthetic construct
  source 1..25: mol_type = other DNA
aaccccagagaagtgcttcattttc

SEQ ID NO: 295 - DNA, 25 bp, synthetic construct
  source 1..25: mol_type = other DNA
gacgaggcttttgagcagctggtgc

SEQ ID NO: 296 - DNA, 25 bp, synthetic construct
  source 1..25: mol_type = other DNA
gcaaaatgcctttacgttgttctaa

SEQ ID NO: 297 - DNA, 25 bp, synthetic construct
  source 1..25: mol_type = other DNA
gggtctggatcttttctcagagcgt

SEQ ID NO: 298 - DNA, 25 bp, synthetic construct
  source 1..25: mol_type = other DNA
ggatttcgtttgctgtggcattggt

SEQ ID NO: 299 - DNA, 25 bp, synthetic construct
  source 1..25: mol_type = other DNA
aacatctgtgtctttattttgctgc

SEQ ID NO: 300 - DNA, 25 bp, synthetic construct
  source 1..25: mol_type = other DNA
tccccagtggtgacctatgtgtgtg

SEQ ID NO: 301 - DNA, 25 bp, synthetic construct
  source 1..25: mol_type = other DNA
atgacagtttcacaacctgcattga

SEQ ID NO: 302 - DNA, 25 bp, synthetic construct
  source 1..25: mol_type = other DNA
gggaacttacttctctatagcccac

SEQ ID NO: 303 - DNA, 25 bp, synthetic construct
  source 1..25: mol_type = other DNA
gctcctagcacgattatatttcacc

SEQ ID NO: 304 - DNA, 25 bp, synthetic construct
  source 1..25: mol_type = other DNA
cactaactatgcatggtgccccagg

SEQ ID NO: 305 - DNA, 25 bp, synthetic construct
  source 1..25: mol_type = other DNA
ggcttgctctgggacttttatgctc

SEQ ID NO: 306 - DNA, 25 bp, synthetic construct
  source 1..25: mol_type = other DNA
gagaataacctcatagtaccaacat

SEQ ID NO: 307 - DNA, 25 bp, synthetic construct
  source 1..25: mol_type = other DNA
ggctccagaacgaagatccacactt

SEQ ID NO: 308 - DNA, 25 bp, synthetic construct
  source 1..25: mol_type = other DNA
ggatttttcttatgcactcctatgc

SEQ ID NO: 309 - DNA, 25 bp, synthetic construct
  source 1..25: mol_type = other DNA
aaacatgtactggcacaatttgtga

SEQ ID NO: 310 - DNA, 25 bp, synthetic construct
  source 1..25: mol_type = other DNA
aaggcagacagttttgctttagtac

SEQ ID NO: 311 - DNA, 25 bp, synthetic construct
  source 1..25: mol_type = other DNA
acgggcctttttcacttagaagctg

SEQ ID NO: 312 - DNA, 25 bp, synthetic construct
  source 1..25: mol_type = other DNA
gagtgtctcagaagtgtgctcctct

SEQ ID NO: 313 - DNA, 25 bp, synthetic construct
  source 1..25: mol_type = other DNA
gctctgtgctttccactatacacag

SEQ ID NO: 314 - DNA, 25 bp, synthetic construct
  source 1..25: mol_type = other DNA
ggttgaaacatatctcttatcttac

SEQ ID NO: 315 - DNA, 25 bp, synthetic construct
  source 1..25: mol_type = other DNA
tggtacttatcagtgccaagcgtcc

SEQ ID NO: 316 - DNA, 25 bp, synthetic construct
  source 1..25: mol_type = other DNA
gtaagagaattatctgcaacttgat

SEQ ID NO: 317 - DNA, 25 bp, synthetic construct
  source 1..25: mol_type = other DNA
ttcatagtcacatcaaggtcatcaa

SEQ ID NO: 318 - DNA, 25 bp, synthetic construct
  source 1..25: mol_type = other DNA
ggaactatctgaaatctgctcagag

SEQ ID NO: 319 - DNA, 25 bp, synthetic construct
  source 1..25: mol_type = other DNA
gcagttgccagaccaataaaatacc

SEQ ID NO: 320 - DNA, 25 bp, synthetic construct
  source 1..25: mol_type = other DNA
gggagataccgaagcctactgtggt

SEQ ID NO: 321 - DNA, 25 bp, synthetic construct
  source 1..25: mol_type = other DNA
tggaagtgtatctcagtcagccagt

SEQ ID NO: 322 - DNA, 25 bp, synthetic construct
  source 1..25: mol_type = other DNA
gatctgtaatgttatttccacttat

SEQ ID NO: 323 - DNA, 25 bp, synthetic construct
  source 1..25: mol_type = other DNA
ttttctactctagcctgttcatatg

SEQ ID NO: 324 - DNA, 25 bp, synthetic construct
  source 1..25: mol_type = other DNA
ggcttcccagataagcagcccagga

SEQ ID NO: 325 - DNA, 25 bp, synthetic construct
  source 1..25: mol_type = other DNA
gtggactaaacctgagaatctcgat

SEQ ID NO: 326 - DNA, 25 bp, synthetic construct
  source 1..25: mol_type = other DNA
gtggtttgacccaagaccacaacca

SEQ ID NO: 327 - DNA, 25 bp, synthetic construct
  source 1..25: mol_type = other DNA
ggtgagctcagatagtctctttctc

SEQ ID NO: 328 - DNA, 25 bp, synthetic construct
  source 1..25: mol_type = other DNA
tcgtctctaaccacgcctattaagc

SEQ ID NO: 329 - DNA, 25 bp, synthetic construct
  source 1..25: mol_type = other DNA
gcataggcagtattttcctgtcagc

SEQ ID NO: 330 - DNA, 25 bp, synthetic construct
  source 1..25: mol_type = other DNA
aatgaggactctctacaggacccga

SEQ ID NO: 331 - DNA, 25 bp, synthetic construct
  source 1..25: mol_type = other DNA
caagtacagcgagttcttcacgggc

SEQ ID NO: 332 - DNA, 25 bp, synthetic construct
  source 1..25: mol_type = other DNA
gatgggacgcataatcattacctta

SEQ ID NO: 333 - DNA, 25 bp, synthetic construct
  source 1..25: mol_type = other DNA
cgatcatctttcctgttccagagag

SEQ ID NO: 334 - DNA, 25 bp, synthetic construct
  source 1..25: mol_type = other DNA
gagctccagagggtccagaaggtcc

SEQ ID NO: 335 - DNA, 25 bp, synthetic construct
  source 1..25: mol_type = other DNA
tcagcaacaactgcctgggggacgc

SEQ ID NO: 336 - DNA, 25 bp, synthetic construct
  source 1..25: mol_type = other DNA
aggggtgataaatgcgttcatgctc

SEQ ID NO: 337 - DNA, 25 bp, synthetic construct
  source 1..25: mol_type = other DNA
tgcgtagattcttgaccatgtagta

SEQ ID NO: 338 - DNA, 25 bp, synthetic construct
  source 1..25: mol_type = other DNA
aagctgtgttcatggccgagaccaa

SEQ ID NO: 339 - DNA, 25 bp, synthetic construct
  source 1..25: mol_type = other DNA
tgttcacggcggattctttgtttta

SEQ ID NO: 340 - DNA, 25 bp, synthetic construct
  source 1..25: mol_type = other DNA
gaacatctttgacactctgcagact

SEQ ID NO: 341 - DNA, 25 bp, synthetic construct
  source 1..25: mol_type = other DNA
ttcccacgggtgccatgaagtgtgt

SEQ ID NO: 342 - DNA, 25 bp, synthetic construct
  source 1..25: mol_type = other DNA
ggtcccaccaacagttcagaaacct

SEQ ID NO: 343 - DNA, 25 bp, synthetic construct
  source 1..25: mol_type = other DNA
gaggcatcaaagcacctgtcgcctc

SEQ ID NO: 344 - DNA, 25 bp, synthetic construct
  source 1..25: mol_type = other DNA
aacattcccttgatctcatcagttc

SEQ ID NO: 345 - DNA, 25 bp, synthetic construct
  source 1..25: mol_type = other DNA
caacaacgaaggacttttctccctg

SEQ ID NO: 346 - DNA, 25 bp, synthetic construct
  source 1..25: mol_type = other DNA
tgtgtagacaccctcttgggtccaa

SEQ ID NO: 347 - DNA, 25 bp, synthetic construct
  source 1..25: mol_type = other DNA
ccacaagaagccttatcctacgtcc

SEQ ID NO: 348 - DNA, 25 bp, synthetic construct
  source 1..25: mol_type = other DNA
tgagtgcacacactaccaatcgttc

SEQ ID NO: 349 - DNA, 25 bp, synthetic construct
  source 1..25: mol_type = other DNA
gatgcggttccgctgcgaagatggg

SEQ ID NO: 350 - DNA, 25 bp, synthetic construct
  source 1..25: mol_type = other DNA
gtcagtagggatgtgtgcctggctg

SEQ ID NO: 351 - DNA, 25 bp, synthetic construct
  source 1..25: mol_type = other DNA
ctctctagttagatatctgacttgg

SEQ ID NO: 352 - DNA, 25 bp, synthetic construct
  source 1..25: mol_type = other DNA
acagggcccagttaatggcagagca

SEQ ID NO: 353 - DNA, 25 bp, synthetic construct
  source 1..25: mol_type = other DNA
gaagcgatgaatttggagactctgt

SEQ ID NO: 354 - DNA, 25 bp, synthetic construct
  source 1..25: mol_type = other DNA
gggtcccagaggaacgctggagcca

SEQ ID NO: 355 - DNA, 25 bp, synthetic construct
  source 1..25: mol_type = other DNA
ggagcacatgttactgcttcatttt

SEQ ID NO: 356 - DNA, 25 bp, synthetic construct
  source 1..25: mol_type = other DNA
atcattattattgctctccactgta

SEQ ID NO: 357 - DNA, 25 bp, synthetic construct
  source 1..25: mol_type = other DNA
catgggaagacgaggccctcgggcc

SEQ ID NO: 358 - DNA, 25 bp, synthetic construct
  source 1..25: mol_type = other DNA
taccagggactatctttgctgcaga

SEQ ID NO: 359 - DNA, 25 bp, synthetic construct
  source 1..25: mol_type = other DNA
gtgaggccacagtcattgaggccct

SEQ ID NO: 360 - DNA, 25 bp, synthetic construct
  source 1..25: mol_type = other DNA
gagcccacttgcattttcatagttt

SEQ ID NO: 361 - DNA, 25 bp, synthetic construct
  source 1..25: mol_type = other DNA
cactgcactgccattgtcttttggt

SEQ ID NO: 362 - DNA, 25 bp, synthetic construct
  source 1..25: mol_type = other DNA
gatcaaagagttcgccgcgggctac

SEQ ID NO: 363 - DNA, 25 bp, synthetic construct
  source 1..25: mol_type = other DNA
aagaaaatcctgagctttcctgtcc

SEQ ID NO: 364 - DNA, 25 bp, synthetic construct
  source 1..25: mol_type = other DNA
gatcgtcgcccgatcagtgttttga

SEQ ID NO: 365 - DNA, 25 bp, synthetic construct
  source 1..25: mol_type = other DNA
gctaccgaggctgtgtgttgaggtc

SEQ ID NO: 366 - DNA, 25 bp, synthetic construct
  source 1..25: mol_type = other DNA
tatcggcctaatcttagccctgaag

SEQ ID NO: 367 - DNA, 25 bp, synthetic construct
  source 1..25: mol_type = other DNA
cgtcgaccaatgccaggattcagag

SEQ ID NO: 368 - DNA, 25 bp, synthetic construct
  source 1..25: mol_type = other DNA
cagacaagcaacccaaactgaaccc

SEQ ID NO: 369 - DNA, 25 bp, synthetic construct
  source 1..25: mol_type = other DNA
tctccttagtcttctcatagcatta

SEQ ID NO: 370 - DNA, 25 bp, synthetic construct
  source 1..25: mol_type = other DNA
ttcaccacctgactttgaatttgtc

SEQ ID NO: 371 - DNA, 25 bp, synthetic construct
  source 1..25: mol_type = other DNA
tggtgaatgtctgttcagctcttct

SEQ ID NO: 372 - DNA, 25 bp, synthetic construct
  source 1..25: mol_type = other DNA
ttccctcgtgacagtggtgtgtggt

SEQ ID NO: 373 - DNA, 25 bp, synthetic construct
  source 1..25: mol_type = other DNA
gacattcttatgcttcttttacaac

SEQ ID NO: 374 - DNA, 25 bp, synthetic construct
  source 1..25: mol_type = other DNA
ccccctttcctacagcaatatgttc

SEQ ID NO: 375 - DNA, 25 bp, synthetic construct
  source 1..25: mol_type = other DNA
agacatcaagtcccattatgccttc

SEQ ID NO: 376 - DNA, 25 bp, synthetic construct
  source 1..25: mol_type = other DNA
gaacaagtgactctgaccagcaggc

SEQ ID NO: 377 - DNA, 25 bp, synthetic construct
  source 1..25: mol_type = other DNA
gacctggctgaaactcttaaaaaag

SEQ ID NO: 378 - DNA, 25 bp, synthetic construct
  source 1..25: mol_type = other DNA
tcttctggagcctgtgtactgcgga

SEQ ID NO: 379 - DNA, 25 bp, synthetic construct
  source 1..25: mol_type = other DNA
gattcccaaacgtggttacattagc

SEQ ID NO: 380 - DNA, 25 bp, synthetic construct
  source 1..25: mol_type = other DNA
agcaagctgccgaaccaaaagaatt

SEQ ID NO: 381 - DNA, 25 bp, synthetic construct
  source 1..25: mol_type = other DNA
ggattctcaatgtataagttgcctt

SEQ ID NO: 382 - DNA, 25 bp, synthetic construct
  source 1..25: mol_type = other DNA
agctactctcaggctgcagtgtgag

SEQ ID NO: 383 - DNA, 25 bp, synthetic construct
  source 1..25: mol_type = other DNA
ttagcctttacctgtgaagtgttac

SEQ ID NO: 384 - DNA, 25 bp, synthetic construct
  source 1..25: mol_type = other DNA
gtttgctctgcatttttgatgatgg

SEQ ID NO: 385 - DNA, 25 bp, synthetic construct
  source 1..25: mol_type = other DNA
ggcctgacgtaattcactcgtttcc

SEQ ID NO: 386 - DNA, 25 bp, synthetic construct
  source 1..25: mol_type = other DNA
ctgcctaccatggtctggggcttga

SEQ ID NO: 387 - DNA, 25 bp, synthetic construct
  source 1..25: mol_type = other DNA
tctgccattgcggctgtcattgcga

SEQ ID NO: 388 - DNA, 25 bp, synthetic construct
  source 1..25: mol_type = other DNA
atcagaccctgacgaagccaagttg

SEQ ID NO: 389 - DNA, 25 bp, synthetic construct
  source 1..25: mol_type = other DNA
gaacatgtgtctatctgcttttgcc

SEQ ID NO: 390 - DNA, 25 bp, synthetic construct
  source 1..25: mol_type = other DNA
tcagggtgcaagctgctggtgaggt

SEQ ID NO: 391 - DNA, 25 bp, synthetic construct
  source 1..25: mol_type = other DNA
ctgcgaggccagggaagccctgagt

SEQ ID NO: 392 - DNA, 25 bp, synthetic construct
  source 1..25: mol_type = other DNA
ttgtctctgcagtgattcggtttct

SEQ ID NO: 393 - DNA, 25 bp, synthetic construct
  source 1..25: mol_type = other DNA
gactggcattttacttctgtgtttt

SEQ ID NO: 394 - DNA, 25 bp, synthetic construct
  source 1..25: mol_type = other DNA
atcattgaggccaggagctctgccc

SEQ ID NO: 395 - DNA, 25 bp, synthetic construct
  source 1..25: mol_type = other DNA
actggctgcgagtgttcctgagagt

SEQ ID NO: 396 - DNA, 25 bp, synthetic construct
  source 1..25: mol_type = other DNA
gtgaagctgaaggggcctgtgtccc

SEQ ID NO: 397 - DNA, 25 bp, synthetic construct
  source 1..25: mol_type = other DNA
tgatcagtgttgttgctctaggaag

SEQ ID NO: 398 - DNA, 25 bp, synthetic construct
  source 1..25: mol_type = other DNA
gtgcatgctagaatttgggacaggc

SEQ ID NO: 399 - DNA, 25 bp, synthetic construct
  source 1..25: mol_type = other DNA
ctctactctttgccgaactatttgt

SEQ ID NO: 400 - DNA, 25 bp, synthetic construct
  source 1..25: mol_type = other DNA
cgccctttcacttagttcagtgcca

SEQ ID NO: 401 - DNA, 25 bp, synthetic construct
  source 1..25: mol_type = other DNA
attggtccaagattccggatcctaa

SEQ ID NO: 402 - DNA, 25 bp, synthetic construct
  source 1..25: mol_type = other DNA
tgttcgtggtgagctacgccaagga

SEQ ID NO: 403 - DNA, 25 bp, synthetic construct
  source 1..25: mol_type = other DNA
cagtattgggtctgcaactcatccg

SEQ ID NO: 404 - DNA, 25 bp, synthetic construct
  source 1..25: mol_type = other DNA
ctgttctgcttcgaagtattcaata

SEQ ID NO: 405 - DNA, 25 bp, synthetic construct
  source 1..25: mol_type = other DNA
ttgtattttacaaatctccctcttc

SEQ ID NO: 406 - DNA, 25 bp, synthetic construct
  source 1..25: mol_type = other DNA
gtgtcataattccccgggcaagggc

SEQ ID NO: 407 - DNA, 25 bp, synthetic construct
  source 1..25: mol_type = other DNA
gttgcattttccaggctgagagctg

SEQ ID NO: 408 - DNA, 25 bp, synthetic construct
  source 1..25: mol_type = other DNA
gttgcctgtcagtgtttacaaacta

SEQ ID NO: 409 - DNA, 25 bp, synthetic construct
  source 1..25: mol_type = other DNA
taaaagggcccacaatgctccaatt

SEQ ID NO: 410 - DNA, 25 bp, synthetic construct
  source 1..25: mol_type = other DNA
accgttatgcattactctgtgtcta

SEQ ID NO: 411 - DNA, 25 bp, synthetic construct
  source 1..25: mol_type = other DNA
gtgtctgtgttccaaccactgaatc

SEQ ID NO: 412 - DNA, 25 bp, synthetic construct
  source 1..25: mol_type = other DNA
gagctatgattgcacactgtactct

SEQ ID NO: 413 - DNA, 25 bp, synthetic construct
  source 1..25: mol_type = other DNA
tagatcgtggcccttcaatttagaa

SEQ ID NO: 414 - DNA, 25 bp, synthetic construct
  source 1..25: mol_type = other DNA
gcaagctcttgtcaaattttagttt

SEQ ID NO: 415 - DNA, 25 bp, synthetic construct
  source 1..25: mol_type = other DNA
ggcagctatcttacagacgcatgaa

SEQ ID NO: 416 - DNA, 25 bp, synthetic construct
  source 1..25: mol_type = other DNA
gtccagcgaatgacgtctgtggcca

SEQ ID NO: 417 - DNA, 25 bp, synthetic construct
  source 1..25: mol_type = other DNA
agtcaatgagtcgcttgtgaattct

SEQ ID NO: 418 - DNA, 25 bp, synthetic construct
  source 1..25: mol_type = other DNA
gagctgcgacaactggatgaacatt

SEQ ID NO: 419 - DNA, 25 bp, synthetic construct
  source 1..25: mol_type = other DNA
tattttggtacctgtgcttgccaca

SEQ ID NO: 420 - DNA, 25 bp, synthetic construct
  source 1..25: mol_type = other DNA
caacacagttttcttgtaccctggg

SEQ ID NO: 421 - DNA, 25 bp, synthetic construct
  source 1..25: mol_type = other DNA
tgaggtcaaattttatcttttcact

SEQ ID NO: 422 - DNA, 25 bp, synthetic construct
  source 1..25: mol_type = other DNA
tcattactttctgcttgaccggaag

SEQ ID NO: 423 - DNA, 25 bp, synthetic construct
  source 1..25: mol_type = other DNA
gagtggatctgcggtgaagccaagc

SEQ ID NO: 424 - DNA, 25 bp, synthetic construct
  source 1..25: mol_type = other DNA
caataagtctttctctccgaaaccg

SEQ ID NO: 425 - DNA, 25 bp, synthetic construct
  source 1..25: mol_type = other DNA
aatgcgggcatttaagtctgtccat

SEQ ID NO: 426 - DNA, 25 bp, synthetic construct
  source 1..25: mol_type = other DNA
aaagccctggtcgatgttggagtgg

SEQ ID NO: 427 - DNA, 25 bp, synthetic construct
  source 1..25: mol_type = other DNA
agaatgatgtccccgttgtatgtat

SEQ ID NO: 428 - DNA, 25 bp, synthetic construct
  source 1..25: mol_type = other DNA
tagaaagtttcttccactctttgac

SEQ ID NO: 429 - DNA, 25 bp, synthetic construct
  source 1..25: mol_type = other DNA
ggagacctggttgtgtgtgtgtgag

SEQ ID NO: 430 - DNA, 25 bp, synthetic construct
  source 1..25: mol_type = other DNA
aagagccacctgcaagatggacacg

SEQ ID NO: 431 - DNA, 25 bp, synthetic construct
  source 1..25: mol_type = other DNA
tgttcgtggtgagctacgccaagga

SEQ ID NO: 432 - DNA, 25 bp, synthetic construct
  source 1..25: mol_type = other DNA
gaattctacatactaaatctctctc

SEQ ID NO: 433 - DNA, 25 bp, synthetic construct
  source 1..25: mol_type = other DNA
gatgagcagacttctcggaattcat

SEQ ID NO: 434 - DNA, 25 bp, synthetic construct
  source 1..25: mol_type = other DNA
aactaggacactaactgggttccat

SEQ ID NO: 435 - DNA, 25 bp, synthetic construct
  source 1..25: mol_type = other DNA
gtcccctgcttgaacactgaagggc

SEQ ID NO: 436 - DNA, 25 bp, synthetic construct
  source 1..25: mol_type = other DNA
aaatgttgctctaatcagattgctt

SEQ ID NO: 437 - DNA, 25 bp, synthetic construct
  source 1..25: mol_type = other DNA
gacacctgtgtttcagcatttggag

SEQ ID NO: 438 - DNA, 25 bp, synthetic construct
  source 1..25: mol_type = other DNA
gacccatgagtggaggaactttcag

SEQ ID NO: 439 - DNA, 25 bp, synthetic construct
  source 1..25: mol_type = other DNA
atgacctgcagaaacagaactgttc

SEQ ID NO: 440 - DNA, 25 bp, synthetic construct
  source 1..25: mol_type = other DNA
gctggcaccttagttgcatgaccac

SEQ ID NO: 441 - DNA, 25 bp, synthetic construct
  source 1..25: mol_type = other DNA
tctgcccaactgtcaaccttgacaa

SEQ ID NO: 442 - DNA, 25 bp, synthetic construct
  source 1..25: mol_type = other DNA
gttataccatttaaagctggcacca

SEQ ID NO: 443 - DNA, 25 bp, synthetic construct
  source 1..25: mol_type = other DNA
aaaggtatctgctgcatcgaacttt

SEQ ID NO: 444 - DNA, 25 bp, synthetic construct
  source 1..25: mol_type = other DNA
ttaagttcattgacaccacctccaa

SEQ ID NO: 445 - DNA, 25 bp, synthetic construct
  source 1..25: mol_type = other DNA
ctgctctggtggtgccgaagggcaa

SEQ ID NO: 446 - DNA, 25 bp, synthetic construct
  source 1..25: mol_type = other DNA
gattgactgtcaggcatggctttgt

SEQ ID NO: 447 - DNA, 25 bp, synthetic construct
  source 1..25: mol_type = other DNA
ctgatttaccctacagcttcaggcc

SEQ ID NO: 448 - DNA, 25 bp, synthetic construct
  source 1..25: mol_type = other DNA
tagcttctggtgttgttcctttact

SEQ ID NO: 449 - DNA, 25 bp, synthetic construct
  source 1..25: mol_type = other DNA
gagctactcacattagatgcatcct

SEQ ID NO: 450 - DNA, 25 bp, synthetic construct
  source 1..25: mol_type = other DNA
gaaagttcagccagaatcttcgtcc

SEQ ID NO: 451 - DNA, 25 bp, synthetic construct
  source 1..25: mol_type = other DNA
atacagcagttcgaaagccgcgtcc

SEQ ID NO: 452 - DNA, 25 bp, synthetic construct
  source 1..25: mol_type = other DNA
ttcatctacctctaccaacggtgga

SEQ ID NO: 453 - DNA, 25 bp, synthetic construct
  source 1..25: mol_type = other DNA
aggaggctccgttttgcaaagtgga

SEQ ID NO: 454 - DNA, 25 bp, synthetic construct
  source 1..25: mol_type = other DNA
ctcagtagtaggtttctgttctatt

SEQ ID NO: 455 - DNA, 25 bp, synthetic construct
  source 1..25: mol_type = other DNA
cattacgggtgttgaaggtgtcttc

SEQ ID NO: 456 - DNA, 25 bp, synthetic construct
  source 1..25: mol_type = other DNA
ctgctagtcttaagaactgctttct

SEQ ID NO: 457 - DNA, 25 bp, synthetic construct
  source 1..25: mol_type = other DNA
ggaaaacgggcagtctgctctgctg

SEQ ID NO: 458 - DNA, 25 bp, synthetic construct
  source 1..25: mol_type = other DNA
gccgttcagcctggttagttttcta

SEQ ID NO: 459 - DNA, 25 bp, synthetic construct
  source 1..25: mol_type = other DNA
gactgtcttgaacatcccaaatgcc

SEQ ID NO: 460 - DNA, 25 bp, synthetic construct
  source 1..25: mol_type = other DNA
tcgcccaccaaccaagtgttacaag

SEQ ID NO: 461 - DNA, 25 bp, synthetic construct
  source 1..25: mol_type = other DNA
cgctttccacagtttgttacctgca

SEQ ID NO: 462 - DNA, 25 bp, synthetic construct
  source 1..25: mol_type = other DNA
ggacaaactgccagttttgtttcct

SEQ ID NO: 463 - DNA, 25 bp, synthetic construct
  source 1..25: mol_type = other DNA
ctctcagatatggcctcttacagta

SEQ ID NO: 464 - DNA, 25 bp, synthetic construct
  source 1..25: mol_type = other DNA
cttaagcaaaatactcccaggtctc

SEQ ID NO: 465 - DNA, 25 bp, synthetic construct
  source 1..25: mol_type = other DNA
gcacagctgcggtgatcgtcatgga

SEQ ID NO: 466 - DNA, 25 bp, synthetic construct
  source 1..25: mol_type = other DNA
gtggtatgcctgccaagagacgcaa

SEQ ID NO: 467 - DNA, 25 bp, synthetic construct
  source 1..25: mol_type = other DNA
tattcttggactgtactcttcgcat

SEQ ID NO: 468 - DNA, 25 bp, synthetic construct
  source 1..25: mol_type = other DNA
gaaatcattgtgtctgcattggtca

SEQ ID NO: 469 - DNA, 25 bp, synthetic construct
  source 1..25: mol_type = other DNA
gtaggggttacaattcacattcctt

SEQ ID NO: 470 - DNA, 25 bp, synthetic construct
  source 1..25: mol_type = other DNA
ggtctaggagatctgtcccttttag

SEQ ID NO: 471 - DNA, 25 bp, synthetic construct
  source 1..25: mol_type = other DNA
ccataccaccactgagatctcattt

SEQ ID NO: 472 - DNA, 25 bp, synthetic construct
  source 1..25: mol_type = other DNA
gaagcgaggcacaggcggtgtggac